(19) United States
(12) Patent Application Publication
(10) Pub. No.: US 2023/0158034 A1
(43) Pub. Date: May 25, 2023

(54) CO-TREATMENT WITH CDK4/6 AND CDK2 INHIBITORS TO SUPPRESS TUMOR ADAPTATION TO CDK2 INHIBITORS

(71) Applicants: Pfizer Inc., New York, NY (US); THE REGENTS OF THE UNIVERSITY OF COLORADO, A BODY CORPORATE, Denver, CO (US)

(72) Inventors: Mansi Arora, Framingham, MA (US); Stephen George Dann, Poway, CA (US); Nichol Lee Goodman Miller, Poway, CA (US); Sabrina Spencer, Boulder, CO (US); Todd Lee VanArsdale, Poway, CA (US)

(73) Assignees: Pfizer Inc., New York, NY (US); The Regents of the University of Colorado, a body corporate, Denver, CO (US)

(21) Appl. No.: 17/995,619

(22) PCT Filed: Apr. 7, 2021
(86) PCT No.: PCT/IB2021/052894

§ 371 Date: Oct. 6, 2022

Related U.S. Application Data
(60) Provisional application No. 63/007,329, filed on Apr. 8, 2020.
(60) Provisional application No. 63/165,049, filed on Mar. 23, 2021.

(51) Int. Cl. A61K 31/519; A61K 31/506; A61P 35/00

(52) U.S. Cl. CPC A61K 31/519; A61K 31/506; A61P 35/00

(57) ABSTRACT

The invention provides a method for treating a disease or disorder, and preferably cancer, comprising administering to a subject in need thereof a therapeutically effective amount of a CDK2 inhibitor, and a therapeutically effective amount of a CDK4/6 inhibitor, wherein the CDK4/6 inhibitor prevents rebound phosphorylation mediated by CDK4 and/or CDK6 in response to the inhibition of CDK2.

### REFERENCE TO SEQUENCE LISTING

This application is being filed electronically via EFS-Web and includes an electronically submitted sequence listing in .txt format. The .txt file contains a sequence listing entitled “PC07258002SEQLISTING_ST25.txt” created on Mar. 26, 2021 and having a size of 17 KB. The sequence listing contained in this .txt file is part of the specification and is herein incorporated by reference in its entirety.

### BACKGROUND OF THE INVENTION

### Field of the Invention

The present invention is related to combinations and methods for treating or ameliorating abnormal cell proliferative disorders, such as cancer, through the co-inhibition of cyclin dependent kinase 2 (CDK2) and cyclin dependent kinases 4 and 6 (CDK4/6). In some embodiments, the combinations and methods provide synergistic co-inhibition of CDK2 and CDK4/6.

### Description of the Related Art

Progression through the cell cycle is carried out by sequential phosphorylation events mediated by Cyclin-Dependent Kinases (CDKs). The timing and specificity of CDK activity throughout the cell cycle is brought about by the rise and fall of various cyclins, which activate CDKs via the formation of heterodimeric complexes. Cells in a resting or quiescent state can be stimulated to enter the cell cycle by mitogens that activate the mitogen-activated protein kinase pathway, leading to the upregulation of D-type Cyclins (Cyclins D1, D2, and D3) (Aktas et al., 1997; Sherr, 1993, 1994). Cyclin Ds then bind CDK4 and CDK6 and initiate phosphorylation of the retinoblastoma protein, Rb. While the nature and effect of CDK4/6-mediated phosphorylation of Rb is under active debate (Chung et al., 2019; Narasimha et al., 2014; Sanidas et al., 2019), the current model states that Rb phosphorylation leads to its inactivation and consequent liberation of the E2F transcription factors which drive the expression of genes needed for S-phase entry, including Cyclin E and Cyclin A (Chellappan et al., 1991; Ohtani et al., 1995). CDK4 and CDK6 show structural and functional homology, and both can phosphorylate Rb (Kato et al., 1993; Meyerson and Harlow, 1994). However, their unique lineage specific expression profile suggests that they are not totally redundant (Hu et al., 2009; Malumbres et al., 2004; Rane et al., 1999).

In quiescent cells stimulated to re-enter the cell cycle by addition of mitogens, CDK4/6 activity is required until Cyclin E activates CDK2, which further phosphorylates Rb, initiating a positive feedback loop that ensures full liberation of E2F and passage through the Restriction Point (defined as the time after which cells become mitogen-independent) (Baldin et al., 1993; Lundberg and Weinberg, 1998; Matsushime et al., 1994; Mittnacht et al., 1994). In asynchronously cycling cells, CDK4/6 is only required during for the first 3-6 hours of G1 phase, as determined by the time after which addition of the CDK4/6 inhibitor, such as palbociclib (IBRANCE®, see also U.S. Pat. Nos. 6,936,612 and RE47,739, incorporated herein by reference) no longer impedes the rise of CDK2 activity and cell-cycle progression (Yang et al., 2017b). Upon S-phase entry, Cyclin E levels decline and CDK2-Cyclin A takes over, which promotes phosphorylation of proteins essential to DNA replication (e.g. Cdc6), DNA repair (e.g. Nbs1), histone synthesis (e.g. NPAT), centrosome duplication (e.g. Nucleophosmin, Mps1), among other processes (Fisk and Winey, 2001; Okuda et al., 2000; Petersen et al., 1999; Wohlbold et al., 2012; Zhao et al., 2000). Finally, CDK1-Cyclin A and CDK1-Cyclin B complexes are activated in late S and G2 phases to drive the transition into and completion of mitosis, respectively (Katsuno et al., 2009) (Lindqvist et al., 2009; Lohka et al., 1988). Given the biological importance of CDK-Cyclin complexes, it is not surprising that these complexes and the proteins that regulate them are often mutated in cancer (Deshpande et al., 2005). Common alterations include loss of Rb function or upregulation/amplification of Cyclin D, Cyclin E, CDK4, and CDK6 (Burkhart and Sage, 2008; Keyomarsi et al., 2002; Khatib et al., 1993; Massague, 2004; Musgrove et al., 2011; Park et al., 2014).

Despite the critical functions of CDKs, with the exception of CDK1, many are dispensable in vivo, suggesting functional compensation between the CDKs. For example, deletion of CDK4 in mice selectively affects proliferation of pancreatic beta cells and pituitary lactotrophs, deletion of CDK6 only affects a subset of hematopoietic cells, and CDK2 loss selectively affects proliferation in germline cells (Malumbres et al., 2004; Moons et al., 2002; Rane et al., 1999). While CDK4/CDK6 double knockout is embryonic lethal in mice due to hematopoietic deficiencies, other tissues showed normal proliferation (Malumbres et al., 2004). Consistently, knockout or knockdown of CDK2 in various cell culture models showed that CDK2 is dispensable for cell proliferation (Tetsu and McCormick, 2003).

While these studies support the idea that CDK2 activity is dispensable for viability and proliferation, it was unclear whether CDK2 was not essential for cell-cycle progression or if compensatory kinases were active in the CDK2-null setting (Berthet et al., 2003). As CDK2/CDK4 double knockout mice were also viable, proliferation in the absence of CDK2 or CDK4 was attributed to compensatory phosphorylation by CDK1 (Malumbres et al., 2004). Indeed, mouse embryos lacking CDK2, CDK3, CDK4, and CDK6 could develop through mid-gestation (Santamaria et al., 2007). The conclusion from these mouse knockout studies was that CDK1 was the only essential CDK in mammalian cells and could drive compensatory phosphorylation of all essential CDK2, CDK4, and CDK6 substrates.

Despite the inessentiality of CDK2 as demonstrated by the above studies, there remains considerable interest in developing small molecule inhibitors that target CDK2 for treating cancers that over-express and are dependent on Cyclin E. These cancers have intrinsic resistance to clinical CDK4/6 inhibitors and are thought to be ‘addicted’ to CDK2 for survival (Caldon et al., 2012). Additionally, in preclinical models, prolonged treatment with CDK4/6 inhibitors (e.g., palbociclib, abemaciclib, ribociclib) leads to acquired resistance through loss of Rb, amplification of CCNE1 leading to upregulation of CDK2/Cyclin E activity, or formation of non-canonical CDK2/Cyclin D1 complexes (Franco et al., 2014; Herrera-Abreu et al., 2016; Yang et al., 2017a). To address this clinical hypothesis, a new ATP-competitive CDK inhibitor PF-06873600 (sometimes referred to herein as PF3600, which is further disclosed in U.S. Pat. No. 10,233,188, each chemical structure and its use being specifically incorporated herein by reference) was recently developed by Pfizer Inc. PF3600 was designed to capture cellular signaling activity of CDK2, 4, and 6 complexes while maintaining a significant potency window over the anti-target CDK1. As can be seen, there exist a long-felt need to better understand the potential therapeutic effects of the combined inhibition of CDK2, 4, and 6, especially in the treatment of abnormal cell proliferative disorders, such as cancer.

Here, the present inventors used single-cell time-lapse imaging together with other traditional techniques to characterize the dynamic effect of CDK2 inhibition on substrate phosphorylation and cell-cycle progression. Using a live-cell sensor for CDK2 activity derived from the C-terminus of DNA helicase B (DHB), a rapid compensatory mechanism that drives CDK2 substrate re-phosphorylation and cell-cycle progression upon CDK2 inhibition was demonstrated. Inhibition of CDK2 leads to an immediate loss of phosphorylation across a wide variety of different CDK2 substrates, as expected. However, compensatory substrate phosphorylation begins rapidly, within 1-2 hours, in a remarkable display of cell adaptation. Surprisingly, co-inhibition of CDK2 and CDK1 does not block the compensatory phosphorylation, whereas co-inhibition of CDK2 and CDK4/6 eliminates the rebound phosphorylation and sends cells into a CDK^{low} non-proliferative state. These results indicate that cells may rapidly adapt to loss of CDK2 activity via compensatory activation of CDK4/6, and that CDK2 inhibitors are poised to act synergistically in combination with therapeutics targeting CDK4/6, including approved CDK4/6 inhibitors.

### BRIEF SUMMARY OF THE INVENTION

The present invention provides, in part, a method for treating a disease or disorder, and preferably cancer, comprising administering to a subject in need thereof a therapeutically effective amount of two or more CDK inhibitors that inhibit the activity of CDK2 and CDK4 and CDK6, or a combination of the same.

The present invention further provides, in part, a method for treating a disease or disorder, and preferably cancer, comprising administering to a subject in need thereof a therapeutically effective amount of two or more CDK inhibitors that inhibit the rebound phosphorylation mediated by CDK4 and/or CDK6 in response to the inhibition of CDK2.

In one aspect the present invention provides a method for treating an abnormal cell proliferative disease or disorder, and preferably cancer, comprising administering to a subject in need thereof a therapeutically effective amount of a CDK2 inhibitor, and a therapeutically effective amount of a CDK4/6 inhibitor, wherein the CDK4/6 inhibitor inhibits the rebound phosphorylation mediated by CDK4 and/or CDK6 in response to the inhibition of CDK2.

In another aspect the present invention provides a method for inhibiting rebound phosphorylation mediated by CDK4 and/or CDK6 in response to the inhibition of CDK2 in a cell, comprising introducing to the cell an amount of a CDK2 inhibitor and an amount of a CDK4/6 inhibitor, wherein the amount of the CDK4/6 inhibitor is effective in inhibiting rebound phosphorylation mediated by CDK4 and/or CDK6 in response to the inhibition of CDK2. In some embodiments, the CDK2 inhibitor is introduced followed by the CDK4/6 inhibitor.

In another aspect the present invention provides a method for treating a disease or disorder, and preferably cancer, comprising administering to a subject in need thereof a therapeutically effective amount of a CDK2 inhibitor, and a therapeutically effective amount of a CDK4/6 inhibitor, wherein the therapeutically effective amounts together are effective in treating the disease or disorder.

In another aspect the present invention provides a method for treating a disease or disorder, and preferably cancer, comprising administering to a subject in need thereof a therapeutically effective amount of a CDK2 inhibitor, and a therapeutically effective amount of a CDK4/6 inhibitor, when rebound phosphorylation mediated by CDK4 and/or CDK6 in response to the inhibition of CDK2 is observed in the subject.

The invention also provides therapeutic methods and uses of treating a disease or disorder, and preferably cancer, comprising administering to a subject in need thereof a therapeutically effective amount of a CDK2 inhibitor, and a therapeutically effective amount of a CDK4/6 inhibitor, in further combination with therapeutically effective amounts of one or more additional anti-cancer agents or palliative agents, wherein the therapeutically effective amounts together are effective in treating the disease or disorder, e.g., cancer.

In another aspect, the invention provides a method for the treatment of a disease or disorder mediated by CDK2, CDK4 and/or CDK6 in a subject, and preferably characterized by rebound phosphorylation mediated by CDK4 and/or CDK6 in response to the inhibition of CDK2 in a subject.

In some embodiments of the methods provided herein, the disease or disorder is cancer that is characterized by amplification or overexpression of cyclin E1 (CCNE1) and/or cyclin E2 (CCNE2). In some embodiments of the methods provided herein, the cancer is characterized by resistance to one or more CDK4/6 inhibitors, for example due to increased cyclin E expression. In other frequent embodiments of the methods provided herein, the cancer is characterized by dependence on CDK2 for tumor cell proliferation.

### BRIEF DESCRIPTION OF THE DRAWINGS

FIG. 1A-G: CDK2 activity is acutely inhibited by PF3600, but phosphorylation rebounds rapidly. (A) Schematic of the CDK2 activity sensor. DHB (DNA-Helicase B fragment) localizes to the nucleus when unphosphorylated; progressive phosphorylation leads to translocation of the sensor to the cytoplasm. NLS, nuclear localization signal; NES, nuclear export signal; S, CDK consensus phosphorylation sites on serine. (B) DHB sensor phosphorylation in MCF10A. Actively proliferating cells (CDK2^{inc}, see Methods) are selected for plotting if they received drug during the time window marked with hashed shading. Cells were selected for plotting if they completed anaphase t hr before drug addition, where t was selected to sample the cell cycle between 25% and 75% of the intermitotic time. Number of single-cell traces: DMSO (121, 92, 99, 71), 25 nM PF3600 (133, 92, 104, 76), 100 nM PF3600 (80, 95, 107, 80), 500 nM (19, 51, 159, 119). (C) CDC6 phosphorylation in MCF10A cells as read out by CDC6-YFP C/N ratio. Cells were imaged, treated, and plotted as in FIG. 1B. Number of single-cell traces: DMSO (83), 100 nM PF3600 (86), 500 nM PF3600 (95). (D), (F) and (G) DHB sensor phosphorylation as in FIG. 1B. Number of single-cell traces for (D) RPE-hTERT: DMSO (69, 74, 100, 97), 25 nM PF3600 (66, 97, 92, 106), 100 nM PF3600 (43, 111, 121, 87), 500 nM PF3600 (25, 151, 231, 182); (F) for MCF7: DMSO (148, 126, 124, 107), 25 nM PF3600 (196, 179, 167, 145), 100 nM PF3600 (172, 198, 188, 161), 500 nM PF3600 (246, 230, 191, 190); (G) for OVCAR3: DMSO (100, 100, 100, 86), 25 nM PF3600 (100, 100, 100, 95), 100 nM PF3600 (62, 100, 100, 100), 500 nM PF3600 (22, 100, 88, 94). (E) DHB sensor phosphorylation in CDK2 analogue sensitive RPE-hTERT treated with DMSO or 10 μM 3 MB-PP1 at the indicated time. Number of single-cell traces: DMSO (133), 10 μM 3 MB-PP1 (104).

FIG. 2A-C: Co-inhibition of CDK4/6 and CDK2 blocks proliferation and compensatory phosphorylation of CDK2 substrates. (A) DHB sensor phosphorylation in CDK2 analogue sensitive RPE-hTERT cells treated with DMSO, 10 μM 3 MB-PP1, 1 μM Palbociclib, or 10 μM 3 MB-PP1+1 μM Palbociclib at the indicated time, as in FIG. 1B. 3 MB-PP1 is used to inhibit CDK2 activity in the CDK2 analogue-sensitive cells. Number of single-cell traces: DMSO (133), 10 μM 3 MB-PP1 (104), 1 μM Palbociclib (146), 10 μM 3 MB-PP1+1 μM Palbociclib (160). DMSO and 10 μM 3 MB-PP1 median traces reproduced from FIG. 1E. (B) DHB sensor phosphorylation in MCF10A cells treated with DMSO, 100 nM PF3600, 5 μM Ribociclib, 100 nM PF3600+5 μM Ribociclib as in FIG. 1B; (C) DHB sensor phosphorylation in MCF10A cells treated with DMSO, 100 nM PF3600, 1 μM Abemaciclib, or 100 nM PF3600+1 μM Abemaciclib as in FIG. 1B. (B) Number of single cell traces, left: DMSO (55), 100 nM PF3600 (53), 5 μM Ribociclib (23), 100 nM PF3600+5 μM Ribociclib (26). (C) Number of single cell traces, right: DMSO (197), 100 nM PF3600 (242), 1 μM Abemaciclib (390), 100 nM PF3600+1 μM Abemaciclib (270).

FIG. 3. DHB sensor phosphorylation in MCF10A cells treated with 1 μM palbociclib or 9 μM RO3306 at the indicated concentrations. Number of single-cell traces: DMSO (39), palbociclib (72) and RO3306 (43). Cells received drug 5-6 hr after anaphase.

FIG. 4A-D: Transient loss and rebound in CDK2 substrate phosphorylation upon CDK2 inhibition. (A) and (B) MCF10A cells were treated with 100 nM PF3600 for indicated times, and fixed and stained for (A) phospho-Rb or (B) phospho-Nucleolin. Hoechst was used to quantify DNA content in individual cells. Mean nuclear signals were quantified for cells with 3-4N DNA content and plotted as probability density histograms on the right. (C) Western blots showing phosphorylation levels of select CDK2 substrates in MCF10A cells after treatment with PF3600 for indicated times. β-tubulin and GAPDH serve as loading controls. Band intensity was quantified and plotted as a bar graph. Data are representative of two biological repeats. (D) Unbiased global analysis of phosphorylated peptides after treatment with PF3600. MCF7 cells were treated with 25 nM PF3600 and resulting modulation of phosphorylated peptides was assessed by proteomics. Shown are significantly modulated phosphorylated peptides containing a minimal CDK consensus motif (SP or TP) after 1 hr treatment (p<0.05) and the fate of those same peptides at 24 hr. Data are plotted relative to DMSO control.

FIG. 5A-D: Palbociclib abolishes compensatory phosphorylation of CDK2 substrates. (A) and (B) DHB sensor phosphorylation in MCF10A, RPE-hTERT, and MCF7 cells treated with DMSO, PF3600, PF3600+9 μM RO3306, or PF3600+1 μM palbociclib. For MCF10A and RPE-hTERT, 100 nM PF3600 was used; for MCF7, 25 nM PF3600 was used. Cells were imaged and plotted as in FIG. 1B. The vertical hashed bars represent time of drug addition. (C) Western blots showing phosphorylation levels of select CDK2 substrates in MCF10A cells after co-treatment with 100 nM PF3600 and 1 μM palbociclib for the indicated times. β-tubulin and GAPDH serve as loading controls. The white bars are reproduced from FIG. 4C. Data are representative of two biological repeats. (D) MCF10A cells were treated with 100 nM PF3600+1 μM palbociclib for the indicated times, and fixed and stained for phospho-Rb or phospho-Nucleolin. Mean nuclear signal was quantified for cells with 3-4N DNA content and plotted as probability density histograms. The shaded histograms represent the phospho-Rb or phospho-NCL distribution after PF3600 mono-treatment at the corresponding time points, reproduced from FIGS. 4A and 4B.

FIG. 6A-F: Knockdown of CDK4/6/cyclin D reduces compensatory phosphorylation of CDK2 substrates. (A) DHB sensor phosphorylation in MCF10A and MCF7 treated with DMSO or PF3600 20 hr after transfection with the following siRNAs: non-targeting, CDK4, CDK6, or CDK4 and CDK6. The vertical black lines mark the time of PF3600 addition (100 nM PF3600 for MCF10A; 25 nM PF3600 for MCF7). (B) DHB C/N single-cell traces for individual MCF10A (top) and MCF7 (bottom) cells plotted in (A). Any further mitoses after drug treatment are noted by the sharp drop in the C/N ratio. Gradual drops in the DHB C/N ratio denote a dephosphorylation of DHB without mitoses. (C) DHB sensor phosphorylation in MCF10A and MCF7 treated with DMSO or PF3600 6 hr after transfection with the following siRNAs: non-targeting, CCND1, CCND2, CCND3, or combined knockdown of CCND1, CCND2, and CCND3 (MCF10A) or CCND1 and CCND3 (MCF7). As MCF7 cells do not express cyclin D2, CCND2 knockdown was omitted from the MCF7 experiment. (D) Western blot analysis of indicated CDKs and D-type cyclins in response to PF3600 treatment (100 nM for MCF10A, 25 nM MCF7) at the indicated times. Whole-cell extracts were analyzed by SDS-PAGE. Control samples are labeled as 0 h. Histone H3 is used as a loading control. (E) Representative mRNA FISH images showing expression of CCND1, CCND2, CCND3, CDK4, and CDK6 mRNA in MCF10A or MCF7 cells in response to PF3600 treatment at the indicated times. Nuclei are stained with Hoechst dye and shown in cyan; mRNA in magenta. (F) Quantification of mRNA FISH data in (E). Error bars indicate standard deviation of multiple images.

FIG. 7A-C: Cdk2 ablation increases sensitivity to palbociclib in Kras^{G12V}/Trp53^{-/-} driven lung tumors. (A) Quantification of average tumor volume fold change (as measured by CT scans) from Kras^{+/LSLG12V}; Trp53^{L/L} mice. Measurements were carried out at 28 days after treatment with palbociclib (70 mg/kg). The number of tumors analyzed in each cohort are specified as ‘n’. Mean tumor volume fold change was calculated as final tumor volume divided by initial tumor volume. Error bars indicate SEM. (B) Western blots of tumors from the Kras^{+/LSLG12V}; Trp53^{L/L} mice treated with palbociclib depicted in FIG. 7A were probed for the indicated biomarkers and quantified. p-values are from a two-sample t-test. (C) Quantification of mean tumor volume fold change from the indicated number of tumors (n) from each cohort of Kras^{+/LSLG12V}; Trp53^{L/L} mice. The Cdk2 status is indicated as wild type (Cdk2^{+/+}) or null (Cdk2^{-/-}). Tumor volumes are measured by CT scans. Mean tumor volume fold change calculated as in A. Error bars indicate SEM.

FIG. 8A-C: (A) MCF10A and MCF7 cells were treated with increasing doses of PF3600 for 1 hr, and phospho-Rb (S807/811) was measured by ELISA. Data represent the mean and standard deviation obtained from duplicate measurements per drug concentration. (B) Density scatter plot of mean nuclear phospho-Rb S807/S811 signal intensities normalized to total Rb in individual MCF10A or MCF7 cells treated with DMSO or 1 μM palbociclib for 1 hr. DNA content was quantified using total nuclear intensity of Hoechst dye. (C) DHB C/N single-cell traces for individual MCF10A, RPE-hTERT, MCF7, and OVCAR3 cells. Cells were selected for plotting if they completed anaphase t hr before drug addition, where t was selected to capture cells that were halfway through the cell cycle (based on inter-mitotic time) at the time of drug addition. Number of single-cell traces: MCF10A: DMSO (53), 25 nM PF3600 (72), 100 nM PF3600 (66). MCF7: DMSO (100), 25 nM PF3600 (100), 100 nM PF3600 (100). RPE-hTERT: DMSO (71), 25 nM PF3600 (68), 100 nM PF3600 (62). OVCAR3: DMSO (19), 25 nM PF3600 (29), 100 nM PF3600 (23). Any additional mitoses after drug treatment are labeled.

FIG. 9A-F: (A) and (B) DHB sensor phosphorylation in wild-type RPE-hTERT (B) or RPE-hTERT cells with a genomic mutation in CDK2 at the gatekeeper residue (RPE-hTERT CDK2^{F80G/F80G}) in both Cdk2 alleles (A). Cells were treated with DMSO or 10 μM of the ATP analog 3 MB-PP1 at the indicated time windows post-anaphase and were imaged and plotted as in FIG. 1B. (C) RPE-hTERT CDK2^{F80G/F80G} were treated with 10 μM 3 MB-PP1 for 1 hr, and fixed and stained with phospho-NBS1 antibody. Histogram of nuclear phospho-NBS1 signal is shown. Data is pooled from two technical replicates. (D) DHB sensor phosphorylation in wild-type RPE-hTERT cells. Cells were treated with 100 nM PF3600 at the indicated time windows post-anaphase and were imaged and plotted as in FIG. 1B. (E) DHB sensor phosphorylation in wild-type RPE-hTERT vs. RPE-hTERT CDK2^{F80G/F80G} cells. Cells were treated with 9 μM RO3306 at the indicated time windows post-anaphase and were imaged and plotted as in FIG. 1B. (F) Protein-normalized phospho-proteomic changes in MCF7 cells treated with 25 nM PF3600 for 1 hr or 24 hr, relative to DMSO control. Each phosphopeptide is normalized to its total cellular levels determined under similar conditions. Black-colored spheres highlight phospho-peptides with a significant decrease at the 1 hr time point, which, in general, return to baseline levels at the 24 hr timepoint.

FIG. 10A-C: (A) MCF10A cells were treated with 100 nM PF3600+9 μM RO3306 for the indicated times, and fixed and stained for phospho-Rb or phospho-Nucleolin. Mean nuclear signal was quantified for cells with 3-4N DNA content and plotted as probability density histograms. The shaded histograms represent the phospho-Rb or phospho-NCL distribution after PF3600 mono-treatment at the corresponding time points, reproduced from FIGS. 4A and 4B. (B) DHB C/N single-cell traces from FIG. 5B for individual MCF10A, RPE-hTERT, and MCF7 cells treated with PF3600 plus palbociclib. (C) Proliferation data corresponding to FIG. 5B for MCF10A, RPE-hTERT, and MCF7 treated as indicated. Cell counts were normalized to the number of cells in the first frame of imaging. Data pooled from three technical replicates. Vertical black line marks the time of drug addition. Note that jogs in cell count can occur upon vehicle or drug addition due to loss of mitotic cells upon media change.

FIG. 11A-F: (A) Western blots validating loss of CDK4 and CDK6 in MCF10A and MCF7 after indicated siRNA treatment. Lysates were collected 24 hr post transfection of siRNAs. β-tubulin or Histone H3 are used as a loading control. (B) DHB sensor phosphorylation in MCF10A or MCF7 cells after treatment with the following siRNAs: non-targeting, CDK4, CDK6, or CDK4 and CDK6. Cells were imaged continuously for 50 hr starting immediately following siRNA transfection. (C) Western blots validating loss of Cyclin D1, D2, D3 in MCF10A and MCF7 after indicated siRNA treatments. Lysates were collected 24 hr post transfection. β-tubulin, GAPDH, or Histone H3 are used as a loading control. (D) DHB sensor phosphorylation in MCF10A or MCF7 cells after treatment with the following siRNAs: non-targeting, CCND1, CCND2, CCND3, simultaneous CCND1, D2, and D3 (MCF10A), or simultaneous CCND1 and D3 (MCF7) knockdown. (E) DHB C/N traces for individual MCF10A (top) and MCF7 (bottom) cells plotted in FIG. 6C. Any further mitoses after drug treatment are noted by the sharp drop in C/N ratios. Gradual drops in the DHB C/N ratio denote a dephosphorylation of DHB without mitosis. (F) Increased Cyclin D3-CDK4 and Cyclin D3-CDK6 protein interaction in MCF10A cells after 24 hr treatment with 100 nM PF3600. CDK-cyclin complexes were immunoprecipitated using antibodies against either CDK4 or CDK6. Rabbit IgG was used for mock immunoprecipitation (IgG). Cyclin D3 and CDK levels in the immunoprecipitates and input (Inp) were determined by western blot.

### DETAILED DESCRIPTION OF THE INVENTION(S)

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

As used herein, “inhibits,” “inhibition” refers to the decrease in activity of a target protein product relative to the normal wild type level. Inhibition may result in a decrease in activity of a target enzyme, and preferably a CDK, and more preferably a decrease in rebound phosphorylation mediated by CDK4 and/or CDK6 in response to the inhibition of CDK2. In some embodiments, the rebound phosphorylation mediated by CDK4 and/or CDK6 is decreased by less than 10%, 10%, 15%, 20%, 25%, 30%, 35%, 40%, 45%, 50%, 55%, 60%, 65%, 70%, 75%, 80%, 85%, 90%, 95% or 100%.

CDKs and related serine/threonine kinases are important cellular enzymes that perform essential functions in regulating cell division and proliferation. “CDK inhibitor” means any compound or agent that inhibits the activity of one or more CDK proteins or CDK/cyclin kinase complexes. The compound or agent may inhibit CDK activity, such as phosphorylation, by direct or indirect interaction with a CDK protein, or it may act to prevent expression of one or more CDK genes. In preferred embodiments, the CDK inhibitors are small molecule CDK inhibitors, or pharmaceutically acceptable salts thereof.

CDK inhibitors include pan-CDK inhibitors that target a broad spectrum of CDKs or selective CDK inhibitors that target specific CDK(s). CDK inhibitors may have activity against targets in addition to CDKs, such as Aurora A, Aurora B, Chk1, Chk2, ERK1 ERK2, GST-ERK1, GSK-3a, GSK-3p, PDGFR, TrkA and VEGFR.

CDK inhibitors include, but are not limited to, abemaciclib (CAS No. 1231929-97-7), alvocidib (i.e., flavopiridol; CAS No. 146426-40-6), dinaciclib (CAS No. 779353-01-4), inditinib (AGM-130; CAS No. 1459216-10-4), milciclib (PHA-848125; CAS No. 802539-81-7), palbociclib (CAS No. 571190-30-2), ribociclib (CAS No. 1211441-98-3), roscovitine (seliciclib; CAS No. 186692-46-6), AT7519 (CAS No. 844442-38-2), AZD5438 (CAS No. 602306-29-6), BMS-265246 (CAS NO. 582315-72-8), BMS-387032 (SNS-032; CAS NO. 345627-80-7), BS-181 (CAS No. 1397219-81-6), FN-1501 (CAS No. 1429515-59-2), JNJ-7706621 (CAS No. 443797-96-4), K03861 (CAS No. 853299-07-7), MK-8776 (CAS No. 891494-63-6), P276-00 (CAS No. 920113-03-7), PF-06873600 (CAS No. 2185857-97-8), PHA-793887 (CAS No. 718630-59-2), R547 (CAS No. 741713-40-6), RO3306 (CAS No. 872573-93-8) and SU 9516 (CAS No. 377090-84-1).

Examples of pan-CDK inhibitors include, but are not limited to, alvocidib, dinaciclib, roscovitine, AT7519, AZD5438, BMS-387032, P276-00, PHA-793887, R547 and SU 9516. A non-limiting example of a selective CDK1 inhibitor is RO3306. Examples of CDK1/2 inhibitors include, but are not limited to, BMS-265246 and JNJ-7706621.

CDK2 inhibitors may be selective or non-selective inhibitors of CDK2. Examples of CDK2 inhibitors include, but are not limited to, K03861, PF-06873600, inditinib, milciclib, and FN-1501. While some compounds, such as PF-06873600, may be identified as CDK2 inhibitors, this designation does not limit the compound's activity towards other CDKs. As such, PF-06873600 may effectively inhibit CDK2, for example in a dose dependent manner, and may also inhibit CDK4 and CDK6, again in some instances in a dose dependent manner (i.e., it may act as a CDK2/4/6 inhibitor). In some embodiments of each of the methods and combinations herein, the CDK2 inhibitor is selected from the group consisting of: 6-(difluoromethyl)-8-[(1R,2R)-2-hydroxy-2-methylcyclopentyl]-2-{[1-(methylsulfonyl)piperidin-4-yl]amino}pyrido[2,3-d]pyrimidin-7(8H)-one (PF-06873600), milciclib, inditinib, and FN-1501, or a pharmaceutically acceptable salt thereof. In some embodiments of each of the methods and combinations herein, the CDK2 inhibitor is selected from the group consisting of: 6-(difluoromethyl)-8-[(1R,2R)-2-hydroxy-2-methylcyclopentyl]-2-{[1-(methylsulfonyl)piperidin-4-yl]amino}pyrido[2,3-d]pyrimidin-7(8H)-one (PF-06873600), inditinib, and FN-1501, or a pharmaceutically acceptable salt thereof. In some embodiments of each of the methods and combinations herein, the CDK2 inhibitor is PF-06873600, or a pharmaceutically acceptable salt thereof.

Examples of selective CDK4/6 inhibitors include, but are not limited to, abemaciclib, ribociclib, palbociclib, lerociclib (CAS No. 1628256-23-4), trilaciclib (CAS No. 1374743-00-6), SHR-6390 (CAS No. 2278692-39-8), and BPI-16350 (CAS No. 2412559-19-2), or a pharmaceutically acceptable salt thereof. In some embodiments of each of the methods and combinations herein, the CDK4/6 inhibitor is selected from the group consisting of: abemaciclib, ribociclib, palbociclib, lerociclib, trilaciclib, SHR-6390, and BPI-16350, or a pharmaceutically acceptable salt thereof. In some embodiments of each of the methods and combinations herein, the CDK4/6 inhibitor is selected from the group consisting of: abemaciclib, ribociclib, and palbociclib, or a pharmaceutically acceptable salt thereof.

In some embodiments of each of the methods and combinations herein, the CDK4/6 inhibitor is palbociclib, or a pharmaceutically acceptable salt thereof.

Preferred examples of CDK4/6 inhibitors and their structures are provided below:

Palbociclib (PD-0332991; IBRANCE®) is a selective CDK4/6 inhibitor sold by Pfizer for the treatment of hormone receptor-positive, HER2-negative metastatic breast cancer in combination with endocrine therapy. The structure of palbociclib is:

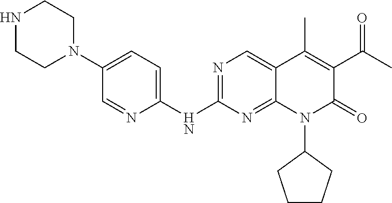 [SMILES: CC(=O)c1c(C)c2cnc(Nc3ccc(N4CCNCC4)cn3)nc2n(C2CCCC2)c1=O]

Abemaciclib (LY2835219; VERZENIO®) is a selective CDK4/6 inhibitor sold by Eli Lilly for the treatment of hormone receptor-positive, HER2-negative metastatic breast cancer in combination with endocrine therapy. The structure of abemaciclib is:

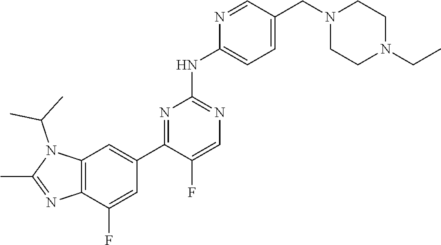 [SMILES: CCN1CCN(Cc2ccc(Nc3ncc(F)c(-c4cc(F)c5nc(C)n(C(C)C)c5c4)n3)nc2)CC1]

Ribociclib (Lee011; KISQALI®), is a selective CDK4/6 inhibitor sold by Novartis for the treatment of hormone receptor-positive, HER2-negative metastatic breast cancer in combination with endocrine therapy. The structure of ribociclib is:

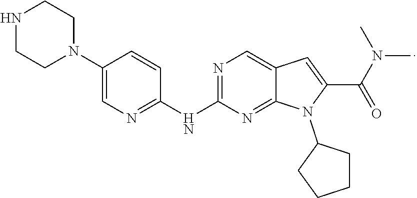 [SMILES: CN(C)C(=O)c1cc2cnc(Nc3ccc(N4CCNCC4)cn3)nc2n1C1CCCC1]

Lerociclib is an oral, selective CDK4/6 inhibitor in clinical development by G1 Therapeutics for use in combination with other targeted therapies in multiple oncology indications. Lerociclib has the structure:

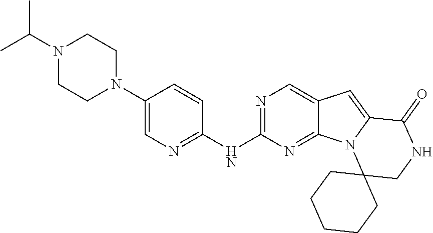 [SMILES: CC(C)N1CCN(c2ccc(Nc3ncc4cc5n(c4n3)C3(CCCCC3)CNC5=O)nc2)CC1]

Trilaciclib is a selective CDK4/6 inhibitor in clinical development by G1 Therapeutics for use in myelopreservation therapy for patients who receive chemotherapy. Trilaciclib is a short-acting intravenous CDK4/6 inhibitor administered prior to chemotherapy and is currently being evaluated clinically. Trilaciclib has the structure:

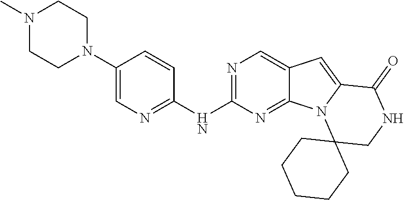 [SMILES: CN1CCN(c2ccc(Nc3ncc4cc5n(c4n3)C3(CCCCC3)CNC5=O)nc2)CC1]

SHR-6390 is a selective CDK4/6 inhibitor being developed by Jiangsu HengRui Medicine Co., Ltd. SHR-6390 is currently being investigated in combination with letrozole or anastrozole or fulvestrant in patients with HR-positive and HER2-negative advanced breast cancer. Various other pyrimidine-based agents have been developed for the treatment of hyperproliferative diseases. U.S. Pat. Nos. 8,822,683; 8,598,197; 8,598,186; 8,691,830; 8,829,102; 8,822,683; 9,102,682; 9,499,564; 9,481,591; and U.S. Pat. No. 9,260,442, filed by Tavares and Strum and assigned to G1 Therapeutics describe a class of N-(heteroaryl)-pyrrolo[3,2-d]pyrimidin-2-amine cyclin dependent kinase inhibitors including those of the formula (with variables as defined therein):

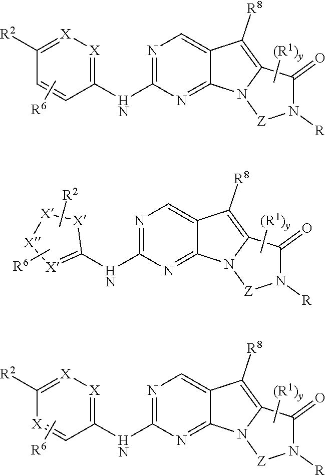

BPI-16350 is a selective CDK4/6 inhibitor being developed by Betta Pharmaceuticals. BPI-16350 is currently being investigated in a Phase I dose escalation study for locally advanced or metastatic solid tumors. BPI-16350 has the structure:

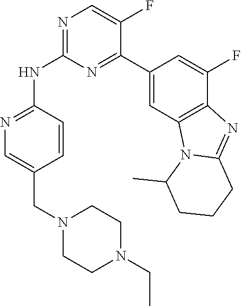 [SMILES: CCN1CCN(Cc2ccc(Nc3ncc(F)c(-c4cc(F)c5nc6n(c5c4)C(C)CCC6)n3)nc2)CC1]

Preferred examples of CDK2 inhibitors and their structures are provided below: The compound 6-(difluoromethyl)-8-[(1R,2R)-2-hydroxy-2-methylcyclopentyl]-2-{[1-(methylsulfonyl)-piperidin-4-yl]amino}pyrido[2,3-d]pyrimidin-7(8H)-one, also referred to herein as PF-06873600 or PF3600, is a CDK2 inhibitor under development by Pfizer that also inhibits CDK4 and CDK6. The chemical structure of PF3600 is identified below and is more fully described in U.S. Pat. No. 10,233,188, which is incorporated herein by reference:

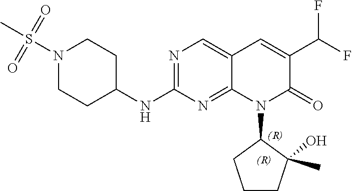

Additional CDK2 inhibitors that may further inhibit other CDKs, e.g., CDK4 and CDK6, include but are not limited to: milciclib, FN-1501, and inditinib (AGM-130). Chemical structures are provided below:

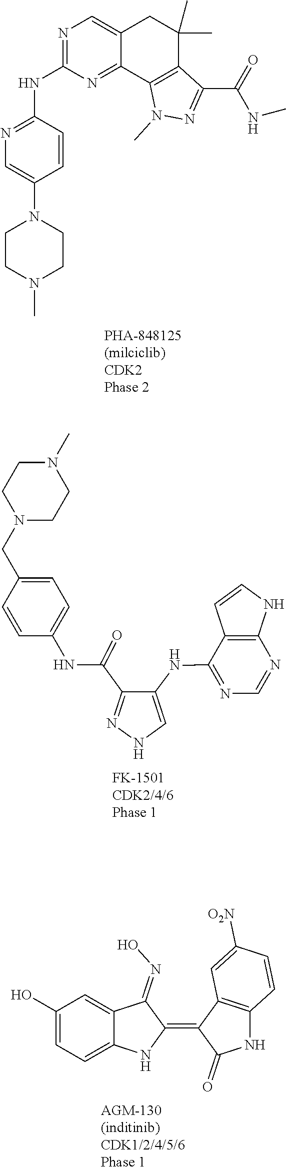 [SMILES: CNC(=O)c1nn(C)c2c1C(C)(C)Cc1cnc(Nc3ccc(N4CCN(C)CC4)cn3)nc1-2 ; CN1CCN(Cc2ccc(NC(=O)c3n[nH]cc3Nc3ncnc4[nH]ccc34)cc2)CC1 ; O=C1Nc2ccc([N+](=O)[O-])cc2/C1=C1/Nc2ccc(O)cc2/C1=N\O]

Unless indicated otherwise, all references herein to small molecule CDK inhibitors, and in particular to small molecule CDK2 inhibitors and CDK4/6 inhibitors, include references to pharmaceutically acceptable salts, solvates, hydrates and complexes thereof, and to solvates, hydrates and complexes of pharmaceutically acceptable salts thereof, and include amorphous and polymorphic forms, stereoisomers, and isotopically labeled versions thereof.

The methods described herein relate to combination therapies comprising administering a CDK2 inhibitor, which may be a selective or non-selective CDK2 inhibitor, and a CDK4/6 inhibitor, which is typically a selective CDK4/6 inhibitor, to a subject in need thereof For clarity, in the methods and combinations described herein it will be understood that the CDK2 inhibitor (i.e., a first CDK inhibitor) and the CDK4/6 inhibitor (i.e., a second CDK inhibitor) are two separate and distinct compounds, not a single compound that inhibits CDK2, CDK4 and CDK6.

In one embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor.

In another embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor. In some embodiments, the CDK2 inhibitor is administered followed by the CDK4/6 inhibitor.

In another embodiment, the invention provides a method for treating a disease or disorder, and preferably cancer, comprising administering to a subject in need thereof a therapeutically effective amount of a CDK2/4/6 inhibitor, and a therapeutically effective amount of a CDK4/6 inhibitor. In some embodiments, the CDK2/4/6 inhibitor is administered followed by the CDK4/6 inhibitor.

In some embodiments of each of the methods and combinations herein, the therapeutically effective amounts of the CDK2 inhibitor and the CDK4/6 inhibitor are together effective in treating the disease or disorder, such as cancer.

In some embodiments of each of the methods and combinations herein, unless otherwise indicated the CDK2 inhibitor may further inhibit CDK4/6 (i.e., a CDK2/4/6 inhibitor).

In another embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor, wherein the CDK4/6 inhibitor prevents rebound phosphorylation mediated by CDK4 and/or CDK6 in response to the inhibition of CDK2.

In another embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor, wherein the amount of the CDK4/6 inhibitor is effective to prevent, ameliorate or reduce rebound phosphorylation mediated by CDK4 and/or CDK6 in response to the inhibition of CDK2.

In another embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor, wherein the CDK4/6 inhibitor prevents rebound phosphorylation mediated by CDK4 and/or CDK6 in response to the inhibition of CDK2. In some embodiments, the CDK2 inhibitor is administered followed by the CDK4/6 inhibitor.

In another embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor, wherein the amount of the CDK4/6 inhibitor is effective to ameliorate or reduce rebound phosphorylation mediated by CDK4 and/or CDK6 in response to the inhibition of CDK2. In some embodiments, the CDK2 inhibitor is administered followed by the CDK4/6 inhibitor.

In another embodiment, the invention provides a method for treating a disease or disorder, and preferably cancer, comprising administering to a subject in need thereof a therapeutically effective amount of a CDK2/4/6 inhibitor, and a therapeutically effective amount of a CDK4/6 inhibitor, wherein the CDK4/6 inhibitor prevents rebound phosphorylation mediated by CDK4 and/or CDK6 in response to the inhibition of CDK2. In some embodiments, the CDK2/4/6 inhibitor is administered followed by the CDK4/6 inhibitor.

In another embodiment, the invention provides a method for treating a disease or disorder, and preferably cancer, comprising administering to a subject in need thereof a therapeutically effective amount of a CDK2/4/6 inhibitor, and a therapeutically effective amount of a CDK4/6 inhibitor, wherein the amount of the CDK4/6 inhibitor is effective to ameliorate or reduce rebound phosphorylation mediated by CDK4 and/or CDK6 in response to the inhibition of CDK2 by the CDK2/4/6 inhibitor. In some embodiments, the CDK2/4/6 inhibitor is administered followed by the CDK4/6 inhibitor.

In one embodiment, the invention provides a method for treating a disease or disorder, and preferably cancer, comprising administering to a subject in need thereof a therapeutically effective amount of a CDK2 inhibitor, and a therapeutically effective amount of a CDK4/6 inhibitor selected from the group consisting of: palbociclib, ribociclib, and abemaciclib, or a pharmaceutically acceptable salt thereof. In preferred embodiments, the CDK4/6 inhibitor is palbociclib or a pharmaceutically acceptable salt thereof.

In one embodiment, the invention provides a method for treating a disease or disorder, and preferably cancer, comprising administering to a subject in need thereof a therapeutically effective amount of a CDK2 inhibitor, wherein the CDK2 inhibitor is PF-06873600 or a pharmaceutically acceptable salt thereof, and a therapeutically effective amount of a CDK4/6 inhibitor selected from the group consisting of: palbociclib, ribociclib, and abemaciclib, or a pharmaceutically acceptable salt thereof. In some embodiments, PF-06873600 or a pharmaceutically acceptable salt thereof is administered followed by the CDK4/6 inhibitor.

In another embodiment, the invention provides a method for treating a disease or disorder, and preferably cancer, comprising administering to a subject in need thereof a therapeutically effective amount of a CDK2 inhibitor, wherein the CDK2 inhibitor is PF-06873600 or a pharmaceutically acceptable salt thereof, and a therapeutically effective amount of a CDK4/6 inhibitor, wherein the CDK4/6 inhibitor is palbociclib or a pharmaceutically acceptable salt thereof.

The invention further provides therapeutic methods and uses comprising administering, a CDK2 inhibitor and a CDK4/6 inhibitor, or pharmaceutically acceptable salts thereof, alone or in combination with one or more other therapeutic agents or palliative agents.

In some embodiments of the methods provided herein, the disease or disorder is abnormal cell growth, in particular cancer. In one aspect, the invention provides a method for the treatment of abnormal cell growth in a subject comprising administering to the subject a therapeutically effective amount of a CDK2 inhibitor and a therapeutically effective amount of a CDK4/6 inhibitor. In frequent embodiments, the abnormal cell growth is cancer. In another aspect, the invention provides a method for the treatment of cancer in a subject comprising administering to the subject an amount of a CDK2 inhibitor and an amount of CDK4/6 inhibitor in further combination with an amount of an additional anti-cancer agent, which amounts are together effective in treating said cancer.

In still another aspect, the invention provides a method for inhibiting cancer cell proliferation in a subject, comprising administering to the subject a CDK2 inhibitor and a CDK4/6 inhibitor in an amount effective to inhibit cancer cell proliferation.

In another aspect, the invention provides a method for inhibiting cancer cell invasiveness in a subject, comprising administering to the subject a CDK2 inhibitor and a CDK4/6 inhibitor in an amount effective to inhibit cancer cell invasiveness.

In another aspect, the invention provides a method for inducing apoptosis in cancer cells in a subject, comprising administering to the subject a CDK2 inhibitor and a CDK4/6 inhibitor in an amount effective to induce apoptosis.

In another aspect, the invention provides a combination comprising a CDK2 inhibitor and a CDK4/6 inhibitor for use in the treatment of cancer in a subject in need thereof. In some such embodiments, the CDK2 inhibitor further inhibits CDK4/6 (i.e., a CDK2/4/6 inhibitor).

In another aspect, the invention provides use of a combination comprising a CDK2 inhibitor and a CDK4/6 inhibitor in the treatment of cancer in a subject in need thereof.

In another aspect, the invention provides use of a combination comprising a CDK2 inhibitor and a CDK4/6 inhibitor in the manufacture of a medicament for the treatment of cancer in a subject in need thereof.

In preferred embodiments of each of the methods, combinations and uses provided herein, the CDK2 inhibitor is PF-06873600 or a pharmaceutically acceptable salt thereof. In preferred embodiments of each of the methods, combinations and uses provided herein, the CDK4/6 inhibitor is palbociclib or a pharmaceutically acceptable salt thereof. In particularly preferred combinations of the methods, combinations and uses provided herein, the CDK2 inhibitor is PF-06873600 or a pharmaceutically acceptable salt thereof and the CDK4/6 inhibitor is palbociclib or a pharmaceutically acceptable salt thereof.

In some embodiments of each of the methods, combinations and uses provided herein, the cancer characterized by resistance to CDK4/6 inhibitors, for example due to increased Cyclin E expression. In other embodiments of each of the methods, combinations and uses provided herein, the cancer characterized by dependence on CDK2 for tumor cell proliferation.

In frequent embodiments of each of the methods, combinations and uses provided herein, the cancer is selected from the group consisting of breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer and thyroid cancer.

In further embodiments of each of the methods, combinations and uses provided herein, the cancer is selected from the group consisting of breast cancer, ovarian cancer, bladder cancer, uterine cancer, prostate cancer, lung cancer, esophageal cancer, liver cancer, pancreatic cancer and stomach cancer. In some such embodiments, the cancer is characterized by dependence on CDK2 for tumor cell proliferation. In other embodiments of the methods provided herein, the abnormal cell growth is cancer characterized by amplification or overexpression of Cyclin E1 (CCNE1) and/or (CCNE2). In some embodiments of each of the methods, combinations and uses provided herein, the subject is identified as having a cancer characterized by amplification or overexpression of CCNE1 and/or CCNE2.

In some embodiments, the cancer is selected from the group consisting of breast cancer and ovarian cancer. In some such embodiments, the cancer is breast cancer or ovarian cancer characterized by amplification or overexpression of CCNE1 and/or CCNE2. In some such embodiments, the cancer is (a) breast cancer or ovarian cancer; (b) characterized by amplification or overexpression of CCNE1 or CCNE2; or (c) both (a) and (b).

In some embodiments, the cancer is ovarian cancer. In some such embodiments, the ovarian cancer is characterized by amplification or overexpression of CCNE1 and/or CCNE2.

In some embodiments, the cancer is breast cancer. In some such embodiments, the breast cancer is hormone receptor positive (HR+), which may be estrogen receptor positive (ER+) and/or progesterone receptor positive (PR+). In some embodiments, the breast cancer is hormone receptor negative (HR−). In some embodiments, the breast cancer is human epidermal growth factor receptor 2 positive (HER2+). In some embodiments, the breast cancer is human epidermal growth factor receptor 2 negative (HER2−). In other such embodiments, the breast cancer is HR-positive, HER2-negative breast cancer; HR-positive, HER2-positive breast cancer; HR-negative, HER2-positive breast cancer; triple negative breast cancer (TNBC); or inflammatory breast cancer. In some embodiments, the breast cancer is endocrine resistant breast cancer, trastuzumab resistant breast cancer, or breast cancer demonstrating primary or acquired resistance to CDK4/6 inhibition. In some embodiments, the breast cancer is advanced or metastatic breast cancer. In some embodiments of each of the foregoing, the breast cancer is characterized by amplification or overexpression of CCNE1 and/or CCNE2.

In some embodiments, the CDK2 inhibitor and the CDK4/6 inhibitor may be administered as first line therapy. In other embodiments, the CDK2 inhibitor and the CDK4/6 inhibitor is administered as second (or later) line therapy. In some embodiments, the CDK2 inhibitor and the CDK4/6 inhibitor are administered as second (or later) line therapy following treatment with an endocrine therapeutic agent. In some embodiments, the CDK2 inhibitor and the CDK4/6 inhibitor are administered as second (or later) line therapy following treatment with an endocrine therapeutic agent. In some embodiments, the CDK2 inhibitor and the CDK4/6 inhibitor are administered sequentially, wherein the CDK2 inhibitor is administered at time-0, followed by administration of the CDK4/6 inhibitor at time-1. In some embodiments, the CDK inhibitors are administered as second (or later) line therapy following treatment with one or more chemotherapy regimens, e.g., including taxanes or platinum agents. In some embodiments, the CDK inhibitors are administered as second (or later) line therapy following treatment with, for example HER2 targeted agents, e.g., trastuzumab.

The term “therapeutically effective amount” as used herein refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

As used herein, “subject” refers to a human or animal subject. In certain preferred embodiments, the subject is a human.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

The term “additional anti-cancer agent” as used herein means any one or more therapeutic agent, other than the combination of the CDK2 and CDK4/6 inhibitors of the invention, that is or can be used in the treatment of cancer, such as agents derived from the following classes: mitotic inhibitors, alkylating agents, antimetabolites, antitumor antibiotics, topoisomerase I and II inhibitors, plant alkaloids, hormonal agents and antagonists, growth factor inhibitors, radiation, inhibitors of protein tyrosine kinases and/or serine/threonine kinases, cell cycle inhibitors, biological response modifiers, enzyme inhibitors, antisense oligonucleotides or oligonucleotide derivatives, cytotoxic agents and immuno-oncology agents.

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one.

Administration of a CDK inhibitor, and preferably administration of a CDK2 inhibitor and a CDK4/6 inhibitor, may be administered by any method that enables delivery of the inhibitors to the site of action. These methods include oral routes, intraduodenal routes, parenteral injection (including intravenous, subcutaneous, intramuscular, intravascular or infusion), topical, and rectal administration. The CDK2 inhibitor and the CDK4/6 inhibitor may be administered sequentially, concurrently or simultaneously. The term “sequential” or “sequentially” refers to the administration of each therapeutic agent of the combination therapy either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly. The term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. The term “simultaneous” refers to the administration of each therapeutic agent of the combination therapy of the invention in the same medicament.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound, for example a CDK2 inhibitor and a CDK4/6 inhibitor, calculated to produce the desired therapeutic effect in association with the required pharmaceutical carrier. The specification for the dosage unit forms are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of a CDK2 inhibitor and a CDK4/6 inhibitor administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day. In one preferred embodiment, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day. In some cases, the aforesaid dosage examples may describe a dosage range for a combination of a CDK2 inhibitor and a CDK4/6 inhibitor. In alternative embodiments, the aforesaid dosage examples may describe dosage ranges for a CDK2 inhibitor, and a CDK4/6 inhibitor individually.

In one preferred embodiment, a therapeutically effective amount or dosage of a CDK4/6 inhibitor may be a dosage sufficient to prevent rebound phosphorylation mediated by CDK4 and/or CDK6 in response to the inhibition of CDK2 by a CDK2 inhibitor.

As used herein, a “pharmaceutically acceptable carrier” refers to a carrier or diluent that does not cause significant irritation to an organism and does not abrogate the biological activity and properties of the administered CDK2 inhibitor and the CDK4/6 inhibitor.

The pharmaceutical acceptable carrier may comprise any conventional pharmaceutical carrier or excipient. The choice of carrier and/or excipient will to a large extent depend on factors such as the mode of administration, the effect of the carrier or excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository. The pharmaceutical composition may be in unit dosage forms suitable for single administration of precise dosages.

Exemplary parenteral administration forms include solutions or suspensions of active compounds in sterile aqueous solutions, for example, aqueous propylene glycol or dextrose solutions. Such dosage forms may be suitably buffered, if desired.

Pharmaceutical compositions suitable for the delivery of compounds of the invention, i.e., the CDK2 and CKD4/6 inhibitors as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The CDK2 and CKD4/6 inhibitors may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The CDK2 and CKD4/6 inhibitors may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrants will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet. Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet. Other conventional ingredients include antioxidants, colorants, flavoring agents, preservatives and taste-masking agents. Exemplary tablets contain up to about 80 wt % drug, from about 10 wt % to about 90 wt % binder, from about O wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated. The formulation of tablets is discussed in detail in “Pharmaceutical Dosage Forms: Tablets, Vol. 1”, by H. Lieberman and L. Lachman, Marcel Dekker, N.Y., N. Y., 1980 (ISBN 0-8247-6918-X), the disclosure of which is incorporated herein by reference in its entirety. Solid formulations for oral administration may be formulated to be immediate and/or modified release. Modified release formulations include delayed-, sustained-, pulsed-, controlled-, targeted and programmed release. Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

The CDK2 and CDK4/6 inhibitors of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art. The solubility of compounds of the invention used in the preparation of parenteral solutions may be increased using appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus, compounds of the invention may be formulated as a solid, semi-solid, or thixotropic liquid for administration as an implanted depot providing modified release of the active compound. Examples of such formulations include drug-coated stents and PGLA microspheres.

The CDK inhibitors of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro needle or needle-free (e.g. Powderject™, Bioject™, etc.) injection. The disclosures of these references are incorporated herein by reference in their entireties. Formulations for topical administration may be formulated to be immediate and/or modified release. Modified release formulations include delayed-, sustained-, pulsed-, controlled-, targeted and programmed release.

The CDK inhibitors of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant known within the art. For intranasal use, the powder may include a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the CDK inhibitors, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the CDK inhibitors of the invention per actuation and the actuation volume may vary from 1 μL to 100 μL. A typical formulation includes one or more CDK inhibitors of the invention, propylene glycol, sterile water, ethanol and sodium chloride. Alternative solvents which may be used instead of propylene glycol include glycerol and polyethylene glycol. Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing, preferably, a desired amount of CDK2 and CDK4/6 inhibitors of the invention The overall daily dose may be administered in a single dose or, more usually, as divided doses throughout the day.

CDK2 and CDK4/6 inhibitors of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

CDK2 and CDK4/6 inhibitors of the invention may also be administered directly to the eye or ear, typically in the form of drops of a micronized suspension or solution in isotonic, pH adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The CDK2 and CDK4/6 inhibitors of the invention and suitable derivatives thereof or polyethylene glycol-containing polymers, in order to improve their solubility, dissolution rate, taste-masking, bioavailability and/or stability for use in any of the modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in PCT Publication Nos. WO 91/11172, WO 94/02518 and WO 98/55148, the disclosures of which are incorporated herein by reference in their entireties.

Inasmuch as it may desirable to administer the combination of a CDK2 inhibitor and a CDK4/6 inhibitor, for example, for the purpose of treating a particular disease or condition such as cancer, it is within the scope of the present invention that a first pharmaceutical composition containing the CDK2 inhibitor and a second pharmaceutical composition containing the CDK4/6 inhibitor, may conveniently be combined in the form of a kit suitable for co-administration of the compositions. Thus, the kit of the invention includes two or more separate pharmaceutical compositions, one of which contains a CDK2 inhibitor and another of which contains a CDK4/6 inhibitor, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

In some aspects, the CDK2 inhibitor and the CDK4/6 inhibitor are part of a combination therapy. As used herein, the term “combination therapy” refers to the administration of a CDK2 inhibitor and a CDK4/6 inhibitor, optionally together with one or more additional pharmaceutical or medicinal agents (e.g., anti-cancer agents), either sequentially, concurrently or simultaneously. The therapeutic effectiveness of the combinations of the invention in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents, such as PD-1 antagonists and the like.

In some embodiments of each of the methods provided herein, the method comprises administering a first CDK inhibitor and a second CDK inhibitor, wherein the first CDK inhibitor is a CDK2 inhibitor (which may be a selective or non-selective inhibitor of CDK2), and the second CDK inhibitor is a CDK4/6 inhibitor, which in preferred embodiments is a selective CDK4/6 inhibitor. Selective CDK inhibitors typically inhibit specific CDK(s) of interest in standard biochemical assays with IC₅₀'s demonstrating at least five-fold selectivity over other CDKs, and preferably ten-fold or greater selectivity over such other CDKs. For example, a selective CDK4/6 inhibitor will typically inhibit CDK4 and CDK6 with at least five- and preferably ten-fold selectivity over other CDKs.

When a combination therapy comprising an additional anti-cancer agent is used, the one or more additional anti-cancer agents may be administered sequentially, concurrently or simultaneously with the CDK2 inhibitor and/or the CDK4/6 inhibitor. In one embodiment, the additional anti-cancer agent is administered to a mammal (e.g., a human) prior to administration of the CDK2 and/or CDK4/6 inhibitors of the invention. In another embodiment, the additional anti-cancer agent is administered to the mammal after administration of the CDK2 and/or CDK4/6 inhibitors of the invention. In another embodiment, the additional anti-cancer agent is administered to the mammal (e.g., a human) simultaneously with the administration of the CDK2 and/or CDK4/6 inhibitors of the invention.

The invention also relates to a pharmaceutical composition for the treatment of abnormal cell growth in a mammal, including a human, which comprises an amount of a CDK2 inhibitor and an amount of a CDK4/6 inhibitor, as defined above (including hydrates, solvates and polymorphs of said compound or pharmaceutically acceptable salts thereof), in combination with one or more (preferably one to three) additional anti-cancer agents.

In particular embodiments, the one or more additional anti-cancer agents are targeted agents, such as inhibitors of PI3 kinase, mTOR, PARP, IDO, TOO, ALK, ROS, MEK, VEGF, FL T3, AXL, ROR2, EGFR, FGFR, Src/Abl, RTK/Ras, Myc, Raf, PDGF, AKT, c-Kit, erbB, CDK2, CDK2/4/6, CDK4/6, CDK5, CDK7, CDK9, SMO, CXCR4, HER2, GLS1, EZH2 or Hsp90, or immunomodulatory agents, such as PD-1 or PD-L 1 antagonists, OX40 agonists or 4-1 BB agonists.

In other embodiments, the one or more additional anti-cancer agents are standard of care agents, such as tamoxifen, docetaxel, paclitaxel, cisplatin, capecitabine, gemcitabine, vinorelbine, exemestane, letrozole, fulvestrant, anastrozole or trastuzumab.

In another embodiment, the invention provides a pharmaceutical composition comprising a CDK2 inhibitor or a pharmaceutically acceptable salt thereof, and a pharmaceutically acceptable carrier or excipient and a pharmaceutical composition comprising a CDK4/6 inhibitor or a pharmaceutically acceptable salt thereof, and a pharmaceutically acceptable carrier or excipient. In some embodiments, the pharmaceutical compositions comprise two or more pharmaceutically acceptable carriers and/or excipients. In other embodiments, the pharmaceutical composition further comprises at least one additional anti-cancer agent.

In some embodiments, a pharmaceutical composition of the invention further comprises at least one additional anti-cancer agent or a palliative agent. In some such embodiments, the at least one additional agent is an anti-cancer agent as described below. In some such embodiments, the combination provides an additive, greater than additive, or synergistic anti-cancer effect.

In one embodiment, the invention provides a method for the treatment of abnormal cell growth in a subject in need thereof, comprising administering to the subject a therapeutically effective amount of a pharmaceutical composition of the invention, or a pharmaceutically acceptable salt thereof.

In another aspect, the invention provides a method for the treatment of abnormal cell growth in a subject in need thereof, comprising administering to the subject an amount of a pharmaceutical composition of the invention, or a pharmaceutically acceptable salt thereof, in combination with an amount of an additional therapeutic agent (e.g., an anticancer therapeutic agent), which amounts are together effective in treating said abnormal cell growth.

In frequent embodiments of the methods provided herein, the abnormal cell growth is cancer. A pharmaceutical composition of the invention may be administered as single agents, for example a pharmaceutical composition of a CDK2 inhibitor, a pharmaceutical composition of a CDK4/6 inhibitor, or a pharmaceutical composition of a CDK2/4/6 inhibitor, or as a single pharmaceutical composition, or may be administered in combination with other anti-cancer agents, in particular standard of care agents appropriate for the particular cancer. In some embodiments, the methods provided result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis.

In another aspect, the invention provides a method for the treatment of a disorder mediated by CDK2, CDK4 and/or CDK6, in a subject, such as certain cancers, comprising administering to the subject a CDK2 inhibitor, or a pharmaceutically acceptable salt thereof, and CDK4/6 inhibitor of the invention, or a pharmaceutically acceptable salt thereof, in an amount that is effective for treating said disorder.

Unless indicated otherwise, all references herein to a CDK inhibitor include references to salts, solvates, hydrates, analogs, and complexes thereof, and to solvates, hydrates and complexes of salts thereof, including polymorphs, stereoisomers, and isotopically labelled versions thereof.

One or more of the CDK inhibitors of the invention may exist in the form of pharmaceutically acceptable salts such as, e.g., acid addition salts and base addition salts of the compounds of one of the CDK inhibitors identified herein. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the CDK inhibitors identified herein.

The invention also relates to prodrugs of the compounds of the formulae provided herein. Thus, certain derivatives of compounds of the invention which may have little or no pharmacological activity themselves can, when administered to a patient, be converted into the inventive compounds, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association), the disclosures of which are incorporated herein by reference in their entireties.

Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety.

All publications and patent applications cited in the specification are herein incorporated by reference in their entirety. It will be apparent to those of ordinary skill in the art that certain changes and modifications may be made thereto without departing from the spirit or scope of the appended claims.

### EXAMPLES

### Example 1: Overview of Rapid Adaptation to CDK2 Inhibition Via CDK4/6-Mediated Rebound Phosphorylation

The present invention demonstrated that CDK2 inhibition leads to rapid and dramatic loss of substrate phosphorylation that is subsequently recovered within several hours. This compensatory phosphorylation phenomenon was observed in multiple cell lines in all phases of the cell cycle. The present invention further demonstrated that cell lines rapidly adapt to loss of CDK2 activity via activation of CDK4 and CDK6 that are active beyond their typical role in early G1. The present invention further demonstrated, using the CDK2/4/6 inhibitor PF3600 and fixed- and live-cell imaging of CDK2 substrates, that in a cell-type-dependent manner, CDK4/6-Cyclin D complexes gave rise to rewiring events that can drive the cell cycle upon inhibition of CDK2, for example in response to a small-molecule CDK2 inhibitor, such as PF3600. This CDK4/6-dependent activity was shown to be full strength by 10 hours after CDK2 inhibition, and may in part be driven by upregulation of CDK4/6/Cyclin D. A striking feature of these findings was the speed with which cells activated bypass mechanisms in response to reduced CDK2 activity.

Notably, since the advent of targeted cancer therapies, significant research effort has been devoted to understanding the molecular mechanisms driving resistance to them. While some cancer drug resistance is driven by pre-existing (intrinsic) genetic resistance to a drug, emerging evidence has demonstrated that non-genetic compensatory mechanisms, including epigenetic changes and activation of bypass pathways, allow cells to counteract targeted therapies (Hata et al., 2016; Ramirez et al., 2016; Shaffer et al., 2017; Sharma et al., 2010). These adaptive responses enable cells to pass through a drug-tolerant state that serves as a reservoir from which cells can acquire bonafide genetic drug resistance mutations. Although the existence of bypass mechanisms has been demonstrated in a variety of cancer types, the reported timescales of adaptation to drug range from weeks to months (Hata et al., 2016; Ramirez et al., 2016; Sharma et al., 2010). Due to the fine time resolution of molecular events demonstrated herein, adaptation to CDK2 inhibition on the timescale of hours was observed. The fact that 100% of cells initially responded to PF3600 argues against intrinsic resistance to the drug, and the rapid timescale of the adaptation argues against acquired genetic mutations as a driver for the observed tolerance to CDK2 inhibitors. Rather, as demonstrated, the data provided herein collectively support rapid upregulation and possible CDK/Cyclin re-complexing as mechanisms for adaptation to CDK2 inhibition, for example after treatment with CDK2 inhibitor drugs.

CDK2/Cyclin complexes phosphorylate numerous substrates involved in critical cellular processes. As such, CDK2 is thought to be a critical regulator of the cell cycle. However, this thinking was challenged a decade and a half ago by mouse and cell-line knockout studies showing that CDK2 was dispensable for development and proliferation (Berthet et al., 2003; Ortega et al., 2003; Santamaria et al., 2007; Tetsu and McCormick, 2003). These studies suggested two possible interpretations: either the CDK2 substrate phosphorylation was not critical to cell cycle progression in the contexts tested or redundant kinase activities could phosphorylate CDK2 substrates in the absence of CDK2 (Berthet et al., 2003; Grim and Clurman, 2003). The data presented herein support the idea that at least a subset of CDK2 substrates are essential for cell-cycle progression and that in the absence of CDK2, CDK4/6 can enable these critical functions in certain cell contexts. Indeed, the data provided herein further indicate that the CDK4/6/Cyclin D complex mediating compensatory phosphorylation may be cell-type specific. This is not surprising given that the different D-type Cyclins, CDK4, and CDK6 are known to have tissue-specific expression and function. However, the kinase/cyclin involved in adaptation to PF3600 is not necessarily the same as the one required for normal cell-cycle progression in a given cell type.

In the widely accepted model of the cell cycle, CDK4/6/Cyclin D complexes function in early G1, after which Cyclin D is degraded and CDK4/6 is rendered inactive (Matsushime et al., 1992). However, a few studies have reported a role for CDK4/6 activity in later cell-cycle stages (Brookes et al., 2015; Gabrielli et al., 1999). Additionally, several studies have reported that Cyclin D1 protein rises in G2 phase of the cell cycle in MCF10A, RPE-hTERT, and MRC5 human fibroblasts, although whether any kinase activity is associated is unknown (Gookin et al., 2017; Yang et al., 2006; Zerjatke et al., 2017). Here it is demonstrated that while CDK4/6 activity appears to be dispensable after G1 phase, CDK4/6 mediates substrate phosphorylation in all cell-cycle phases upon CDK2 inhibition. An apparent delay in CDK4/6 reactivation can be seen in the fact that in one embodiment, co-treatment of PF3600 and palbociclib does not cause an immediate drop of the DHB signal to baseline. Instead, the DHB signal first rises for approximately 5 hr (in parallel with the DHB signal of cells treated with PF3600 alone) before beginning a decline that requires another 5 hr to fall to baseline. The apparent delay in CDK4/6 reactivation may be attributed to the time it takes to upregulate the proteins involved in the compensatory kinase activity.

The CDK4/6-mediated compensatory phosphorylation of CDK2 substrates may be through a direct, or indirect process. For example, in vitro kinase assays utilizing purified CDK/cyclin complexes and purified DHB sensor showed phosphorylation of DHB by CDK2/Cyclin E1, CDK2/Cyclin A2, CDK1/Cyclin A2, and CDK1/Cyclin E1 but not by CDK1/Cyclin B1, CDK4/Cyclin D1, or CDK6/Cyclin D1 (Schwarz et al., 2018; Spencer et al., 2013). However, these assays used tagged and purified CDK/cyclin complexes expressed under normal CDK2-proficient conditions. The CDK/cyclin complexes therefore would not contain post-translational modifications or new protein-protein interactions that may be induced by CDK2 inhibition and that may be necessary to activate CDK4/6/Cyclin D. It is also certainly possible that CDK4/6 enables CDK2 substrate re-phosphorylation via indirect effects by activating other kinases or inhibiting phosphatases, or that CDK2 itself becomes reactivated.

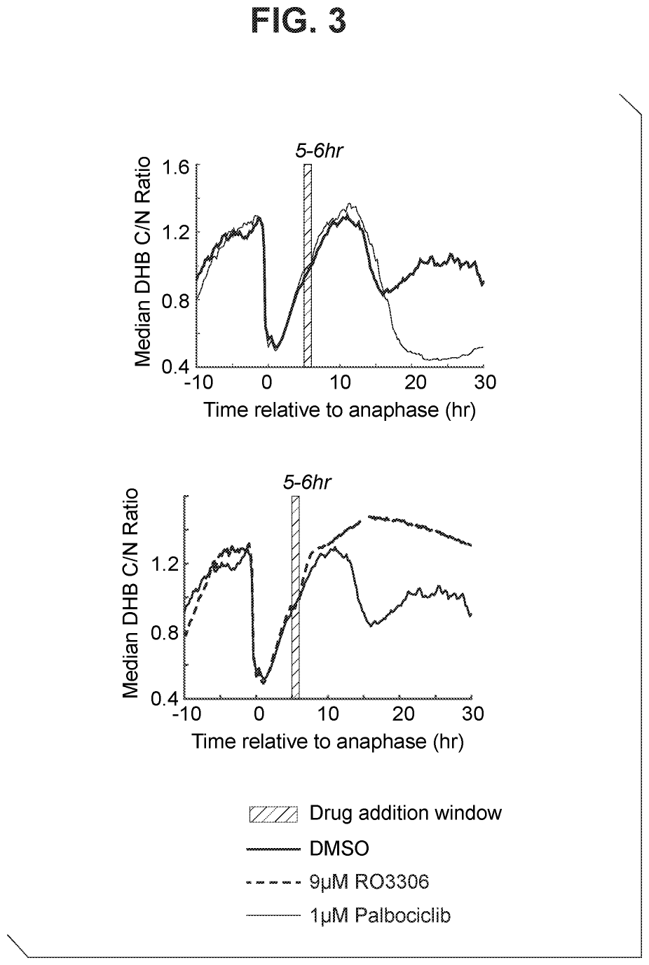

CDK1 performs non-redundant functions during the cell cycle and is thereby considered to be essential. Consistent with this, CDK1 knockout embryos failed to develop (Santamaria et al., 2007) and small molecule inhibition of CDK1 in cell culture results in a G2 arrest and a blockade of mitosis. CDK1 was sufficient for mice to survive in the absence of CDK2 and CDK4 through mid-gestation, after which the embryos died owing to severe hematopoietic defects (Santamaria et al., 2007). It was thus implied that CDK1 could carry out all compensatory kinase activities in the absence of CDK2 and CDK4 in most tissue types. In contrast, here we show that while CDK1 is still essential for entry into mitosis (FIG. 3, right), upon acute CDK2 inhibition in a CDK2-functional background, it plays only a minor compensatory role in phosphorylation of the CDK2 substrates in the cell contexts tested here.

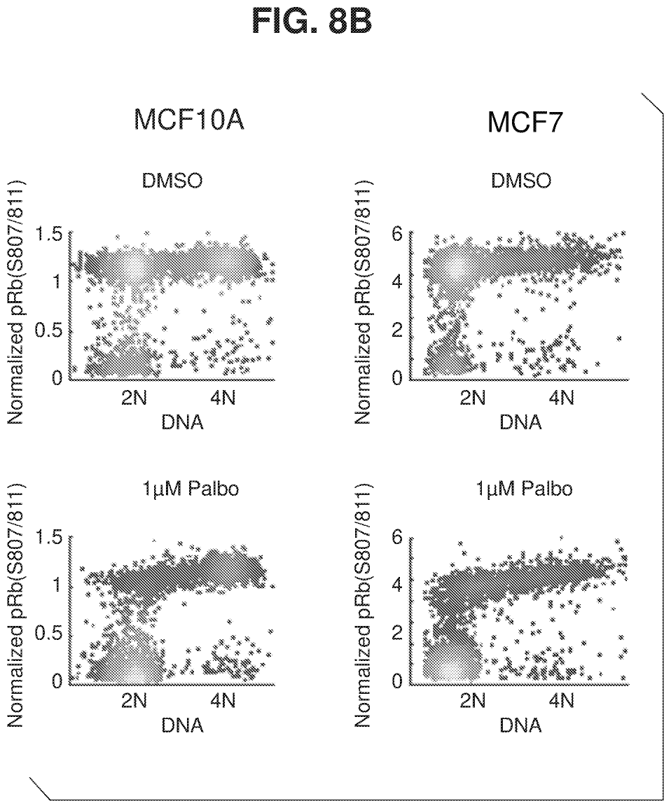

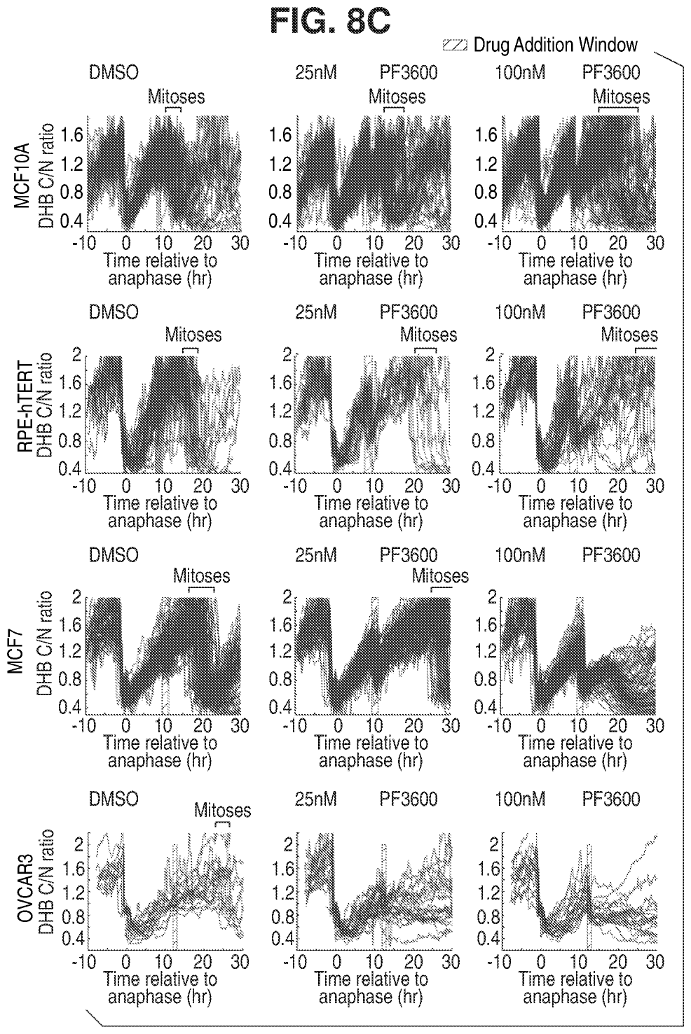

Direct evidence for CDK1 driving compensatory phosphorylation was only shown in mice with all other interphase CDKs completely ablated (CDK2/3/4/6 quadruple knockouts), in contrast to enzymatic inhibition shown here. Interestingly, in CDK4/CDK2 double knockout mice, an increased interaction between CDK6 and cyclin D2 was observed, and it was speculated that CDK6/Cyclin D could drive compensatory phosphorylation in the absence of CDK4 and CDK2 (Barriere et al., 2007). Furthermore, MEFs lacking CDK2 or CDK2 and CDK4 proliferated less efficiently as compared to their wildtype counterparts (Barriere et al., 2007; Berthet et al., 2003; Ortega et al., 2003). Consistent with this, in the present invention longer intermitotic times were observed when CDK2 activity was acutely inhibited using PF3600 in MCF10A, MCF7, and RPE-hTERT cells (FIG. 8C).

The data provided herein suggest that selective CDK2 inhibition may be a promising strategy as a targeted therapy in cancers that have become resistant to clinical CDK4/6 inhibitors due to increased Cyclin E expression, in cancers that depend on CDK2 for tumor cell proliferation, or in cancers that cannot compensate by upregulation of compensatory kinases. In agreement with this idea, OVCAR3 cells are resistant to palbociclib due to Cyclin E amplification but are particularly sensitive to CDK2 inhibition and do not show compensatory phosphorylation of substrates or undergo any further mitoses in response to PF3600. Furthermore, genetically engineered, palbociclib-resistant mouse lung tumors demonstrated combinatorial activity of CDK2 loss and CDK4/6 inhibition similar to inhibition of CDK2/4/6 via PF3600. Those tumors that adapted to CDK2 inhibition via CDK4/6 may be candidates for combination treatment with CDK2 and CDK4/6 inhibitors.

### Example 2: Inhibition of CDK2 Activity Caused a Rapid Loss of Substrate Phosphorylation

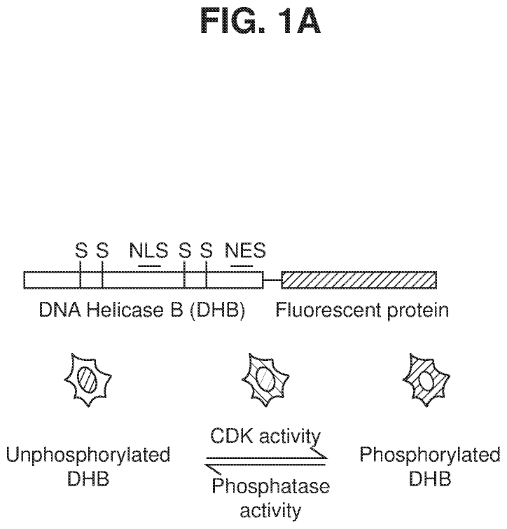

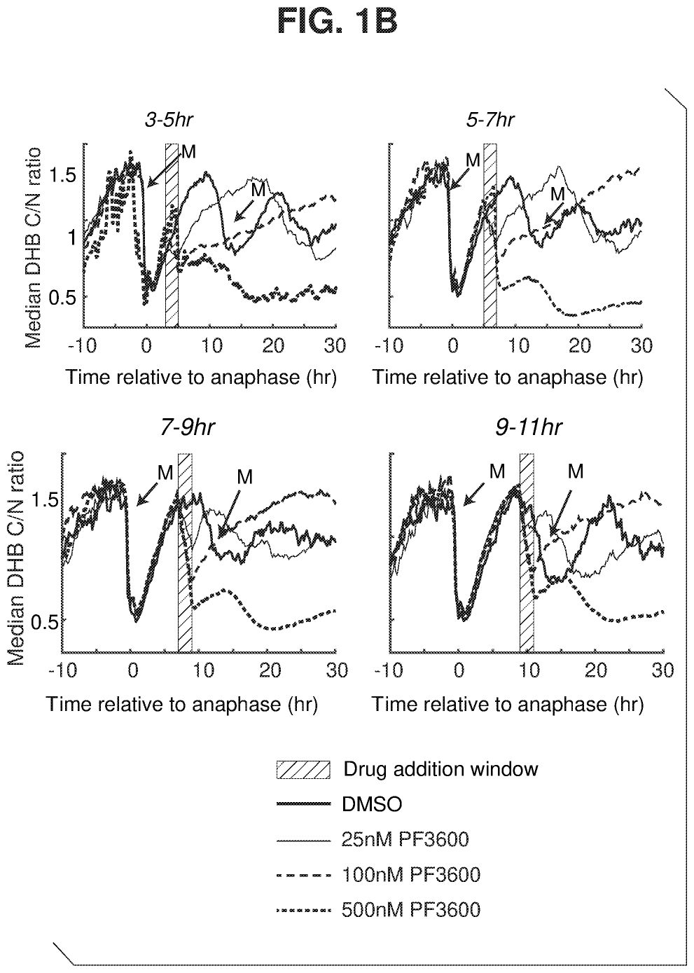

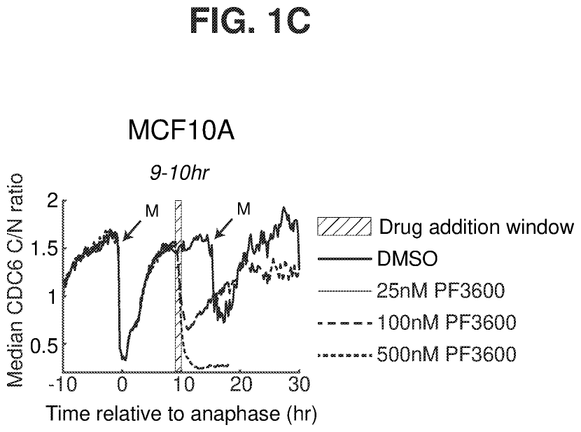

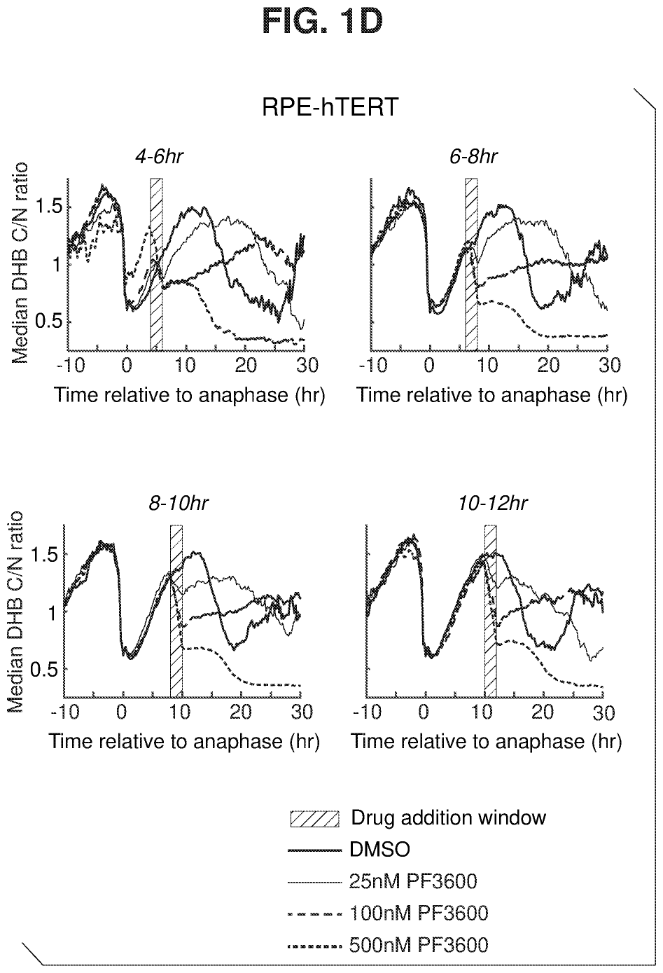

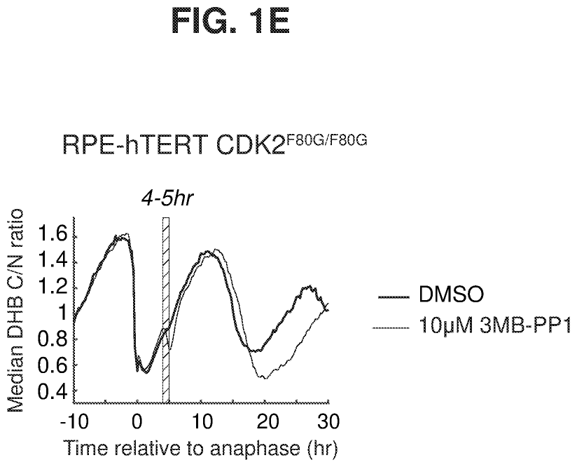

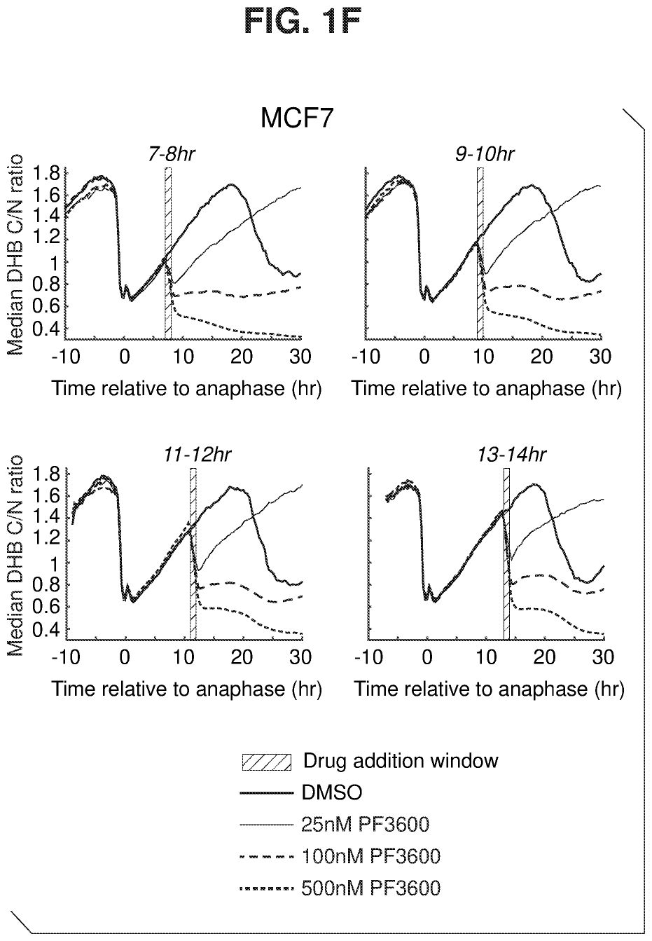

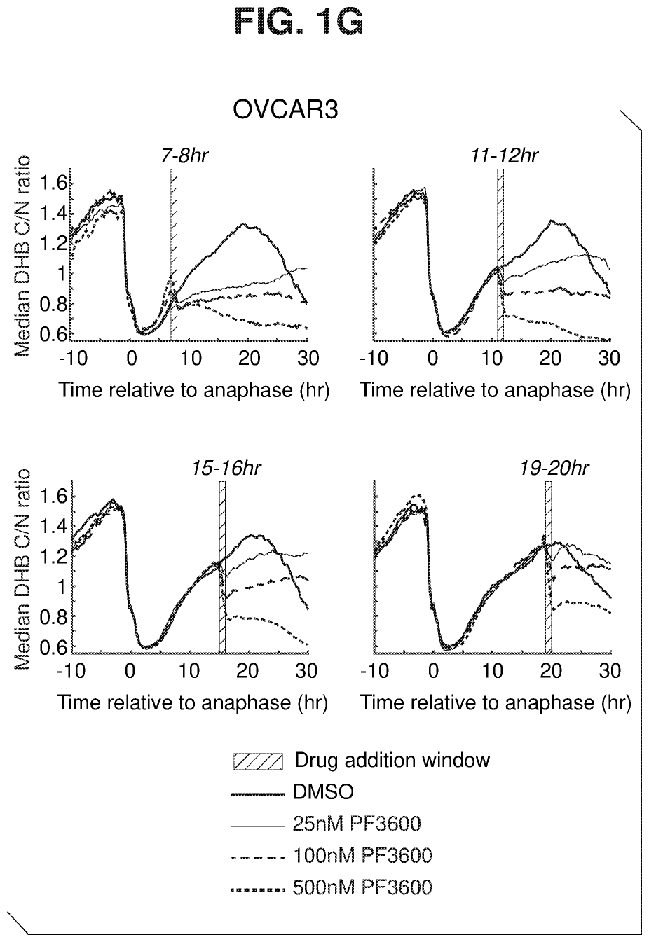

The real-time effects of CDK2 inhibition with PF3600 treatment were first examined using a DHB-based CDK2 activity sensor (Spencer et al., 2013) (FIG. 1A). The DHB sensor is localized to the nucleus when it is not phosphorylated. Upon phosphorylation, the nuclear localization signal is masked, the nuclear export signal is unmasked, and the sensor steadily translocates to the cytoplasm in response to increasing CDK2 activity (FIG. 1A). Thus, kinase activity can be quantified by the ratio of the cytoplasmic to nuclear fluorescence intensity (C/N ratio) of the DHB sensor. In the present invention, cellular IC₅₀ values (FIG. 8A) were used to select 25 nM and 100 nM as relevant doses of PF3600, and time-lapse imaging of the DHB sensor was performed in two non-transformed epithelial cell lines (MCF10A and RPE-hTERT), a breast cancer cell-line (MCF7), and an ovarian cancer line (OVCAR3). Asynchronously cycling cells were first imaged for approximately 20 hr in the absence of drug to establish the cell-cycle phase of each cell; the movie was then paused to add drug, and then imaging was continued for another 1-2 days. Because the cells were cycling asynchronously, all cell-cycle phases with one drug treatment were sampled. This allowed the present inventors to computationally isolate traces for cells that received drug at any time since the completion of anaphase.

At higher concentrations, PF3600 inhibits CDK4/6 in addition to CDK2. To ensure that the effects of PF3600 were primarily due to inhibition of CDK2 without interference from CDK4/6 inhibition, the analyses were restricted to cell-cycle stages where CDK4/6 was thought to be inactive. Consistent with the notion that CDK4/6 acts primarily in G1 phase of the cell cycle (Chung et al., 2019; Sherr and Roberts, 2004) addition of palbociclib 5 hr after anaphase or later had no effect on DHB sensor phosphorylation, cell-cycle progression, or the timing of mitosis (FIG. 3, left). The present inventors therefore reasoned that any changes in DHB phosphorylation in response to PF3600 beginning 5 hr after anaphase would be due to inhibition of CDK2 activity. Similarly, a 1 hr palbociclib treatment resulted in dephosphorylation of Rb in cells with 2N DNA content but had no effect on Rb phosphorylation in cells with 3-4N DNA content (FIG. 8B), again consistent with Chung et al., 2019. Hence, for all experiments assessing CDK2 substrate phosphorylation after treatment with PF3600, analysis was restricted to cells with 3-4N DNA content or those treated ≥5 hr after anaphase.

As anticipated, addition of PF3600 mid-cell cycle led to a sharp and rapid drop in the C/N ratio of the DHB sensor in all four cell lines tested (FIGS. 1B, D, F and G). The real-time effects of PF3600 on phosphorylation of another CDK2 substrate, CDC6, a component of the pre-replication complex that also translocates from the nucleus to the cytoplasm in response to CDK2 phosphorylation (Petersen et al., 1999; Saha et al., 1998), were also examined. Addition of PF3600 led to a drop in CDC6 phosphorylation causing its translocation back to the nucleus (FIG. 1C). Taken together, the drop in the C/N ratio of both DHB and CDC6 suggests rapid (within 1 hr) inhibition of CDK2 activity upon treatment with PF3600.

In assessing the specificity of the DHB sensor, treatment with a high dose of 1 μM palbociclib 5 hr or later after mitosis had no immediate effect on DHB sensor phosphorylation, cell-cycle progression, or the timing of mitosis (FIG. 3, left). Upon completion of the upcoming mitosis, however, these palbociclib-treated cells entered a CDK2^{low} G0 arrest, indicating the effectiveness of palbociclib at inhibiting CDK4/6 and blocking commitment to the subsequent cell cycle. CDK1 inhibition with 9 μM RO3306 also had minimal immediate effect on DHB sensor phosphorylation (FIG. 3, right). Toward the end of the cell cycle, these RO3306-treated cells showed a G2 arrest and plateauing of DHB phosphorylation, indicating the effectiveness of RO3306 at inhibiting CDK1 and blocking mitotic entry. These observations, together with previously published in vitro kinase data (Schwarz et al., 2018; Spencer et al., 2013), and the fact that CyclinE1^{-/-}E2^{-/-}A1^{-/-}A2^{-/-} MEFs maintain nuclear DHB sensor (Chung et al., 2019), demonstrate that the DHB sensor is phosphorylated primarily by CDK2, with minimal phosphorylation by CDK4, CDK6, or CDK1 under normal growth conditions.

### Example 3: Rapid Rebound in CDK2 Substrate Phosphorylation after CDK2 Inhibition

In addition to the immediate drop in DHB sensor phosphorylation upon treatment with PF3600, a rapid rebound in phosphorylation that begins within 1-2 hr was noted in MCF10A, MCF7, and RPE-hTERT cells (FIGS. 1B, 1F and 1D, respectively). By 5 hr, DHB sensor phosphorylation returned to pre-treatment levels, and continued to rise thereafter. Consistent with this, cell-cycle progression continued, and cells eventually completed mitosis (FIG. 8C). MCF7 breast cancer cells were more sensitive to PF3600 than MCF10A and RPE-hTERT cells (FIGS. 1F, 8A, and 8C). While 25 nM PF3600 showed the drop-rebound behavior in MCF7, 100 nM PF3600 caused a brief rebound followed by long-lived suppression of DHB phosphorylation and blocked mitosis (FIGS. 1F and 8C). A drop-rebound was also observed with CDC6 phosphorylation in MCF10A cells treated with PF3600 (FIG. 1C). The ˜30-hour half-life of PF3600 in cells and the pharmacokinetic and pharmacodynamic studies indicated that the observed re-phosphorylation of CDK2 substrates upon PF3600 treatment was not due to instability of the compound or loss of inhibitor binding.

The three cell lines in which the rebound in sensor phosphorylation was observed (MCF10A, MCF7, and RPE-hTERT) were all dependent on CDK4/6/Cyclin D for cell-cycle entry and were, as expected, palbociclib-sensitive. In contrast, OVCAR3 cells have amplification of cyclin E and are resistant to palbociclib. The present inventors hypothesized that if OVCAR3 cells were more reliant on CDK2 activity for their survival and proliferation, these cells would exhibit greater sensitivity to CDK2 inhibition by PF3600 as visualized with the DHB sensor. Consistent with this idea, treatment of OVCAR3 cells with the lower 25 nM dose of PF3600 inhibited cell proliferation and prevented all further mitoses for the remainder of the imaging period (FIGS. 1G and 8C). Interestingly, unlike MCF10A, MCF7, or RPE-hTERT cells, DHB sensor phosphorylation did not rebound in OVCAR3 cells after PF3600 treatment but instead dropped and then reached a plateau at an intermediate level (FIG. 1G).

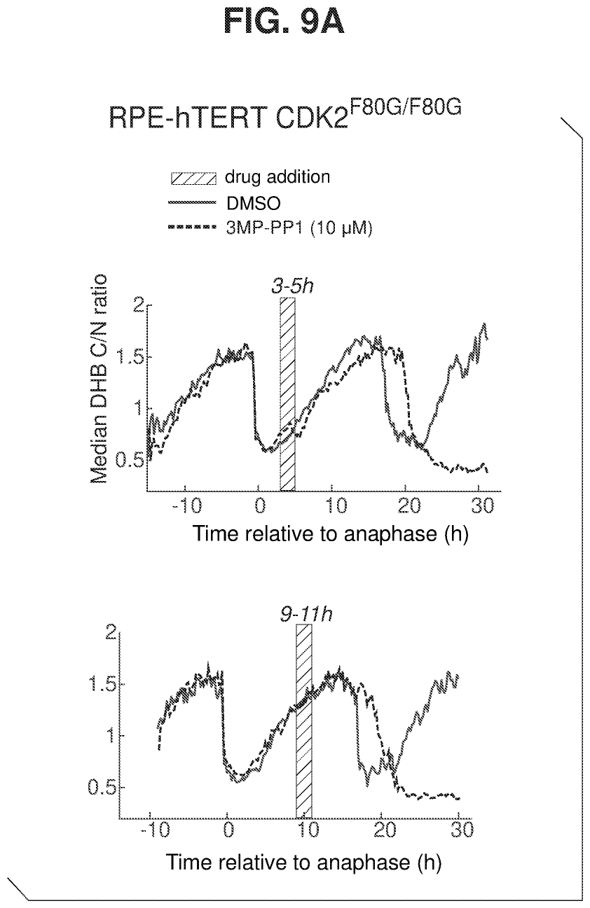

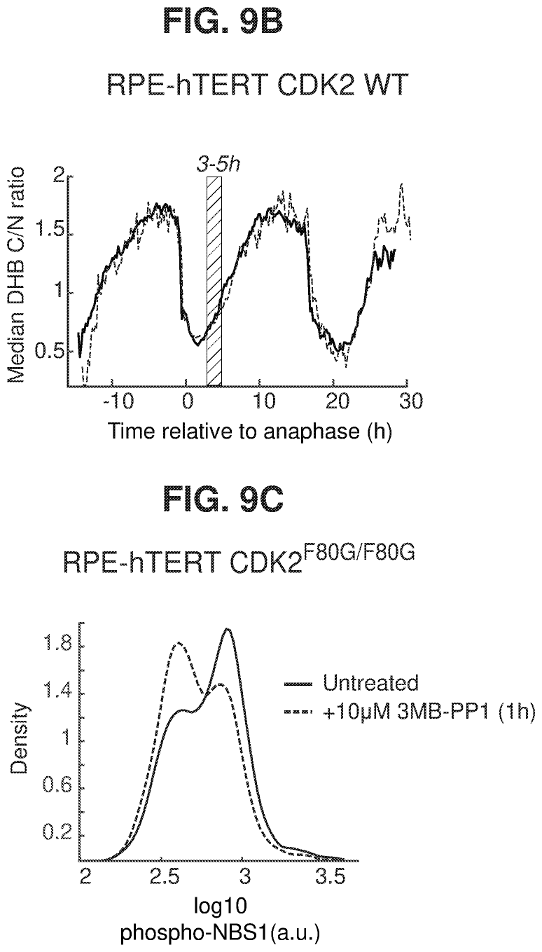

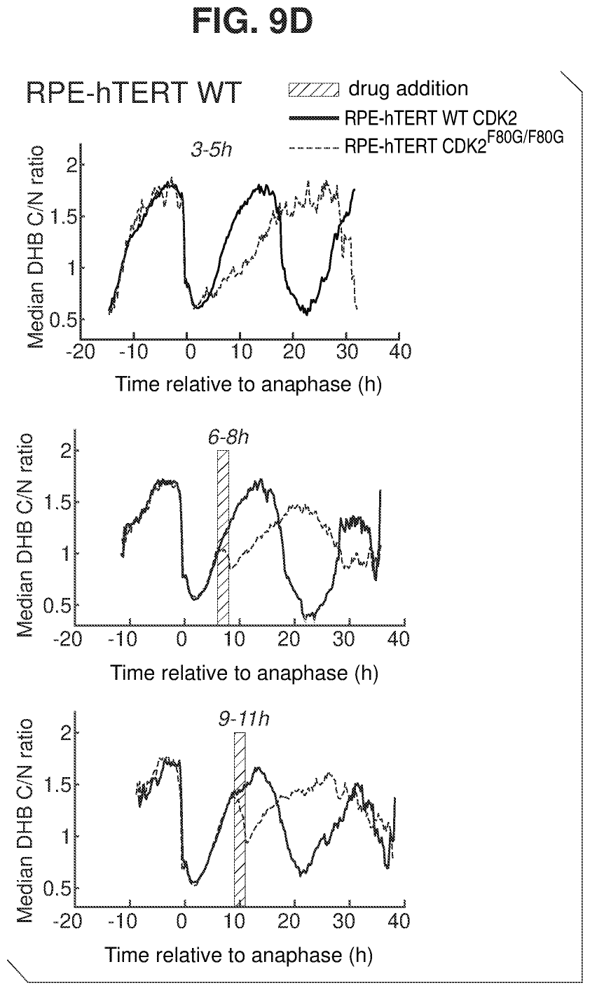

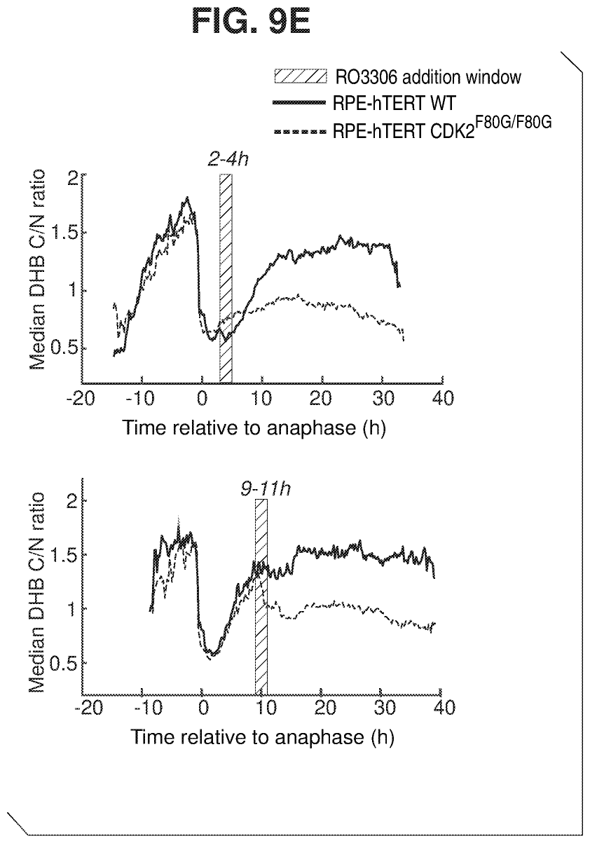

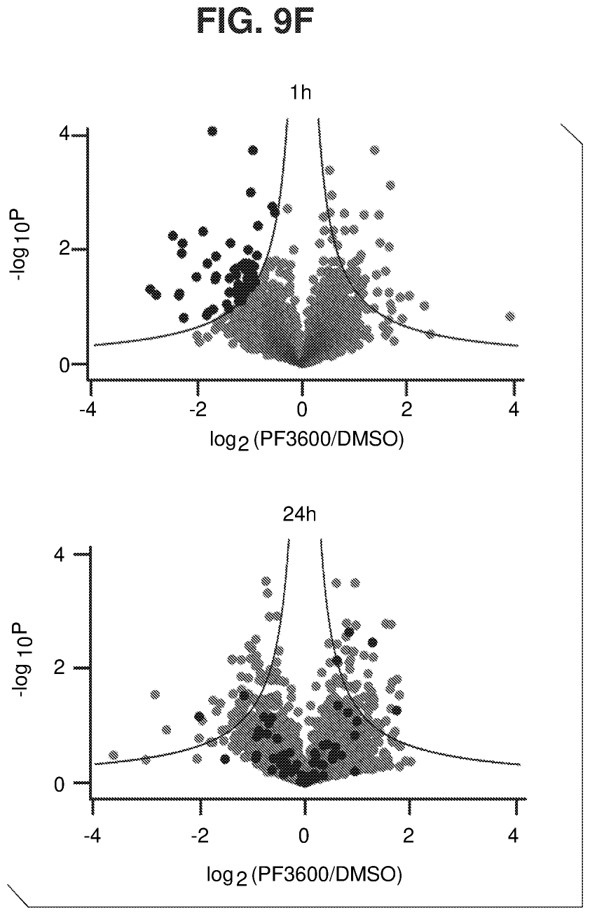

To test the drop-rebound effect in an orthogonal manner, the DHB sensor was transduced into CDK2-analog-sensitive RPE-hTERT cells containing a F80G mutation at both CDK2 alleles (CDK2^{F80G/F80G}; (Merrick et al., 2011)). This mutation creates a modified ATP-binding pocket that is specifically inhibited by a bulky ATP-competitive analog, 3 MB-PP1, 1-(tert-butyl)-3-(3-methylbenzyl)-1H-pyrazolo[3,4-d]pyrimidin-4-amine (CAS No. 956025-83-5). Consistent with PF3600 treatment, inhibition of CDK2 activity by 3 MB-PP1 caused a reduction in DHB sensor phosphorylation that quickly rebounded, whereas wild-type RPE-hTERT cells were not affected by 3 MB-PP1 (FIGS. 1E, 9A and 9B). While the drop in DHB phosphorylation in CDK2^{F80G/F80G} cells with 3 MB-PP1 was notably less dramatic than wild-type RPE-hTERT cells with PF3600, CDK2^{F80G/F80G} was indeed inhibited, as demonstrated by loss of Nbs1 phosphorylation (FIG. 9C). The small effect on DHB phosphorylation with 3 MB-PP1 might be due to the fact that CDK2^{F80G/F80G} cells rely more on CDK1 activity compared to wild-type RPE-hTERT cells, given that mutation of the gatekeeper residue is known to reduce CDK2 function (Merrick et al., 2011). Addition of the CDK1 inhibitor RO3306 led to a much greater drop in DHB phosphorylation in RPE-hTERT CDK2^{F80G/F80G} cells as compared to wild-type RPE-hTERT cells (FIG. 9E). Thus, in RPE-hTERT CDK2^{F80G/F80G} cells, CDK1 is active unusually early in the cell cycle and contributes to phosphorylation of CDK2 substrates, thereby muting the inhibition of DHB phosphorylation attainable with 3 MB-PP1.

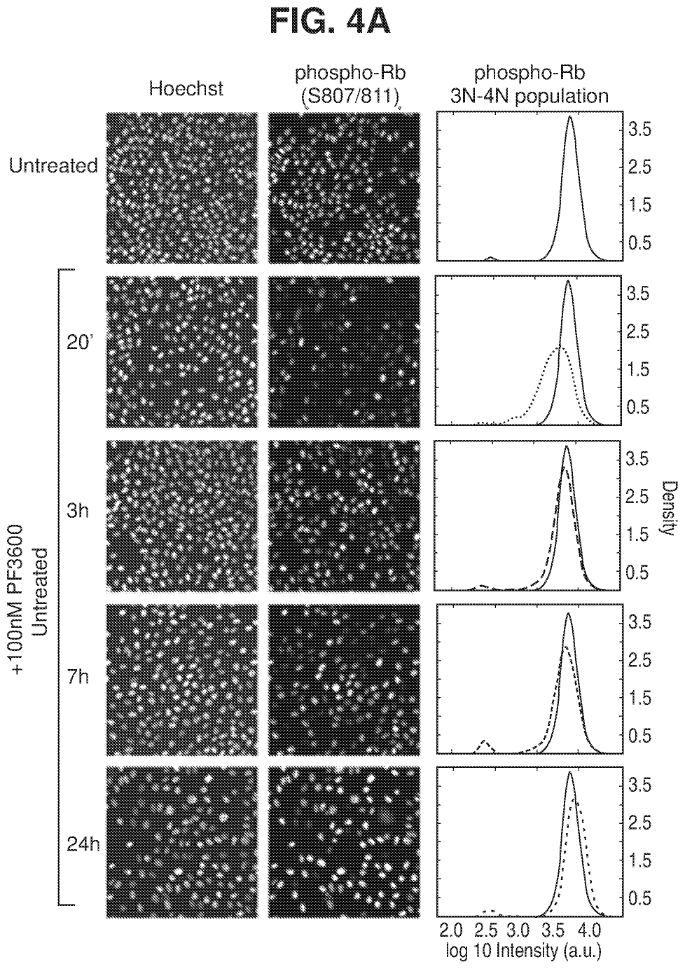

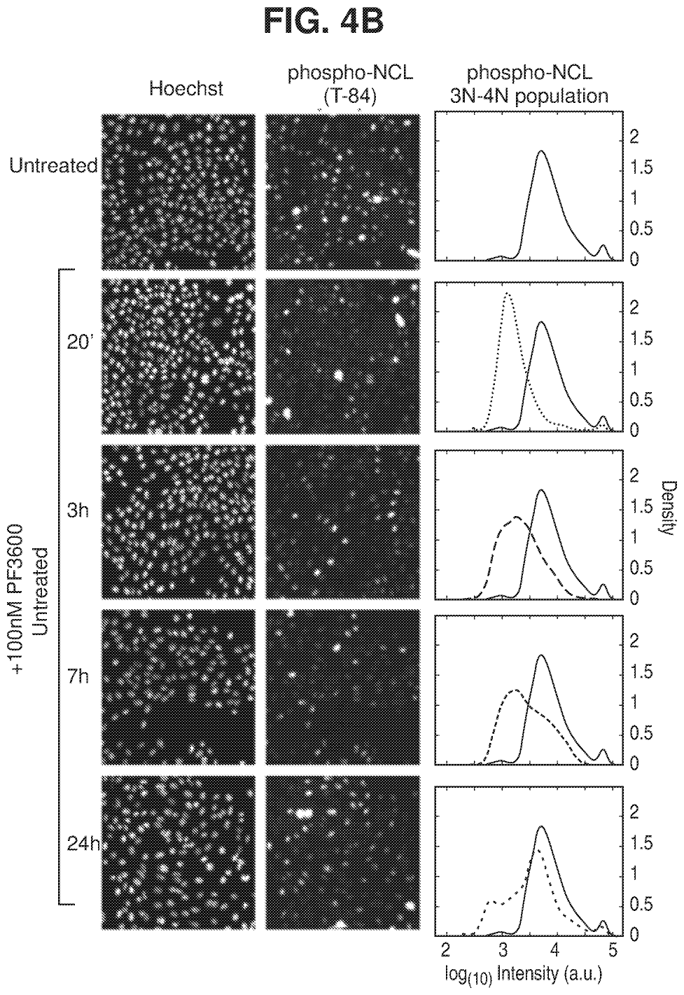

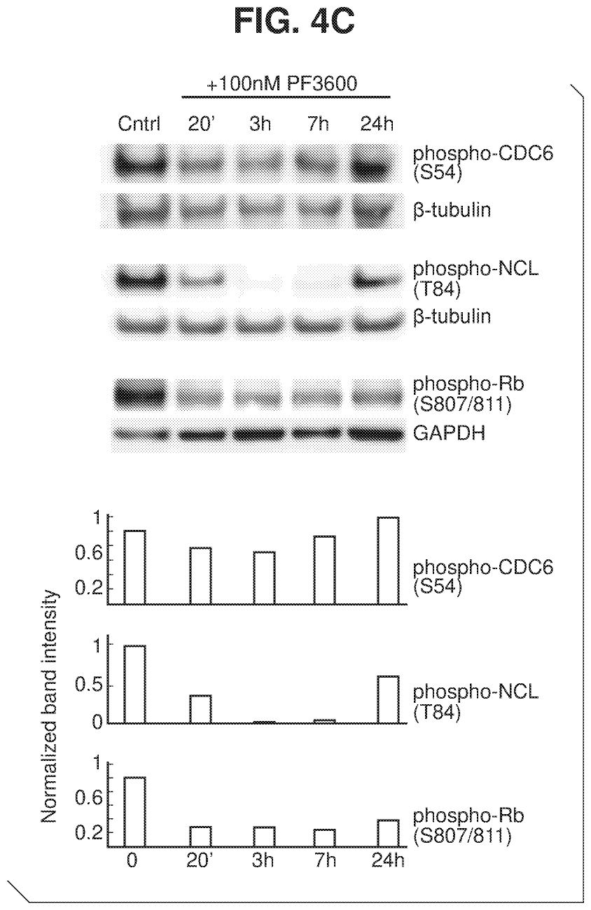

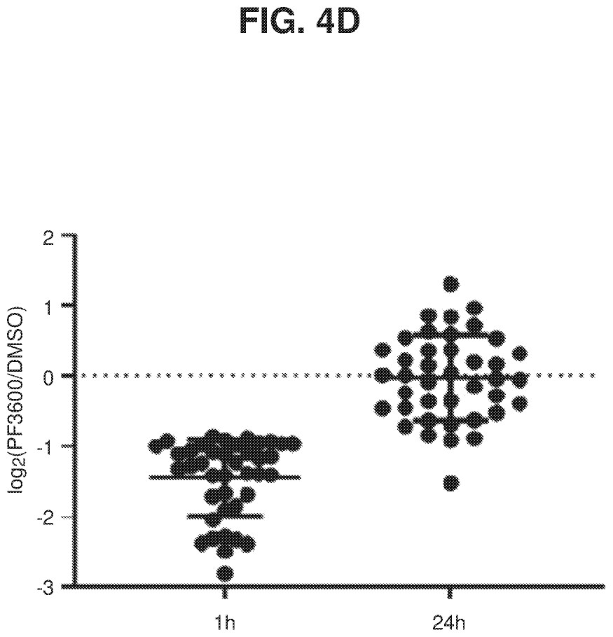

Next, immunofluorescence and western blotting were used to investigate whether CDK2 inhibition had similar effects on the phosphorylation kinetics of endogenous CDK2 substrates: Cdc6 (Petersen et al., 1999; Saha et al., 1998), Nucleolin (Sarcevic et al., 1997), and Rb (Akiyama et al., 1992). The effects of PF3600 on cells with 3-4N DNA content were quantified by immunofluorescence and PF3600 treatment led to a transient reduction in Rb and Nucleolin phosphorylation followed by a rebound (FIGS. 4A and 4B). Similar results were obtained by western blotting for Cdc6, Rb, and Nucleolin (FIG. 4C).

CDK2 substrate dynamics were globally assessed through phospho-proteomics. MCF7 cells were treated with 25 nM PF3600 for 1 hr or 24 hr and the effect on CDK substrate phosphorylation was assessed by mass spectrometry analysis (FIG. 9F). Considering only peptides with a minimal CDK consensus phosphorylation site (SP or TP), 40 peptides were identified whose phosphorylation was significantly reduced (p<0.05) after 1 hr of PF3600 treatment, and the vast majority of these rebounded to control levels by 24 hr (FIG. 4D, FIG. 9F, and Table 1). Taken together, the observations made from live-cell imaging, immunofluorescence, western blotting, and phospho-proteomics suggest that when CDK2 activity was acutely inhibited, cancer cells, such as MCF10A, MCF7, and RPE-hTERT cells rapidly adapted by activation of a compensatory kinase(s) that now phosphorylates CDK2 substrates to facilitate eventual completion of the cell cycle.

### Example 4: Investigation of Compensatory Kinase Activity: Contribution of CDK1 was Minor

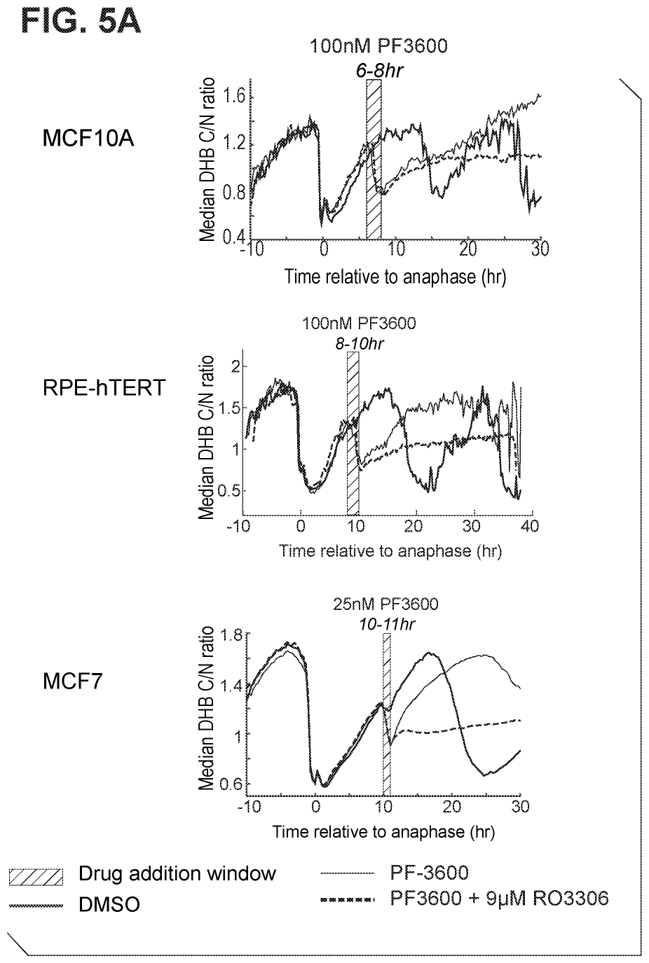

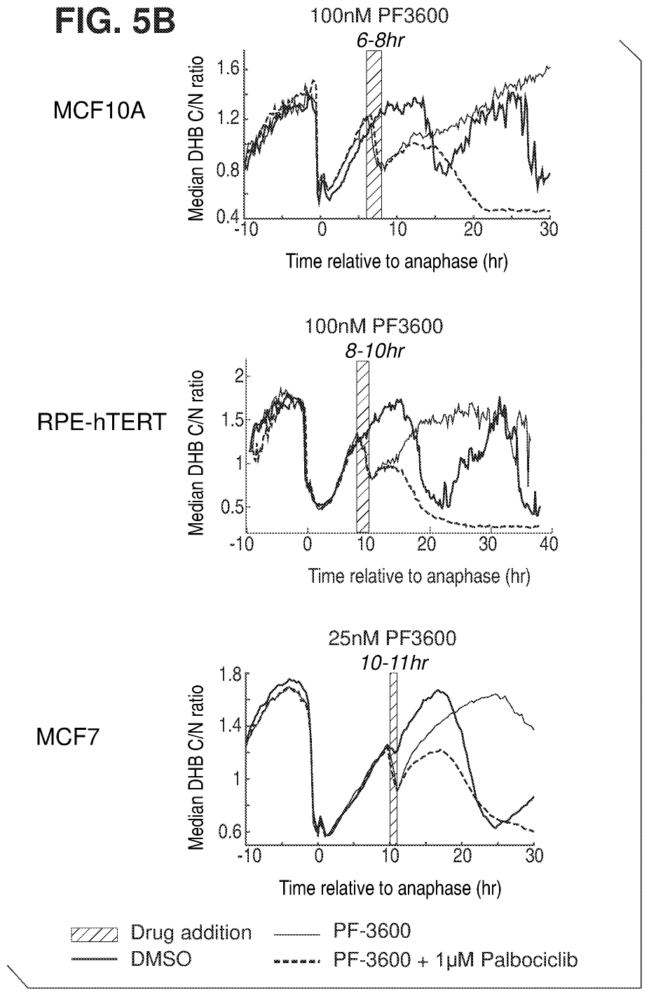

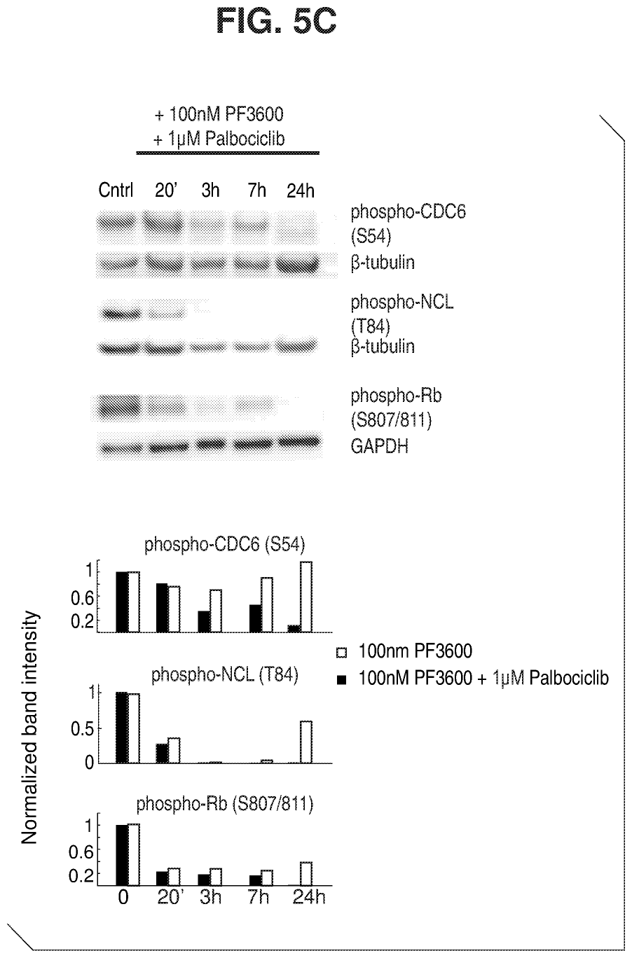

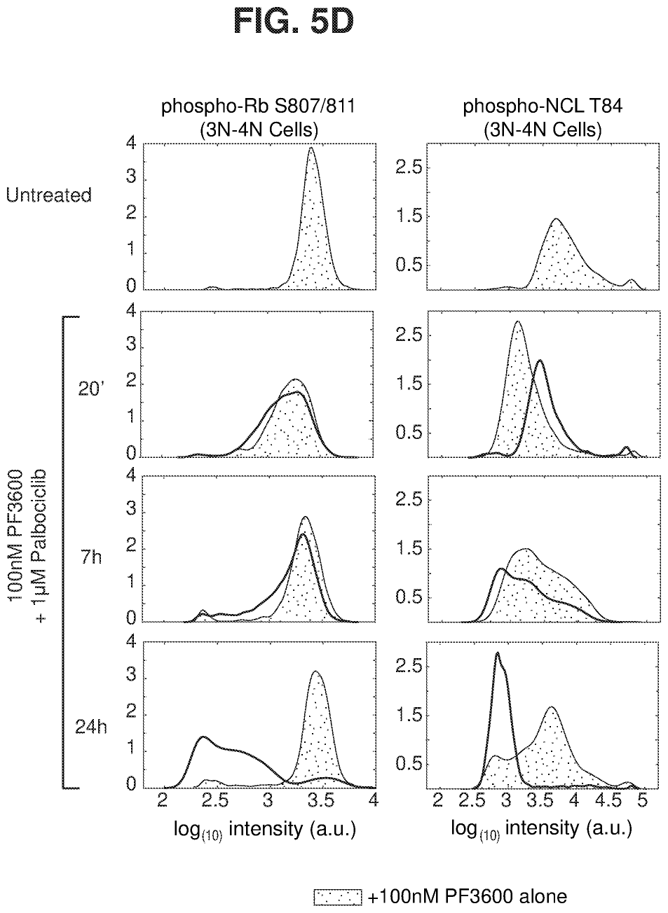

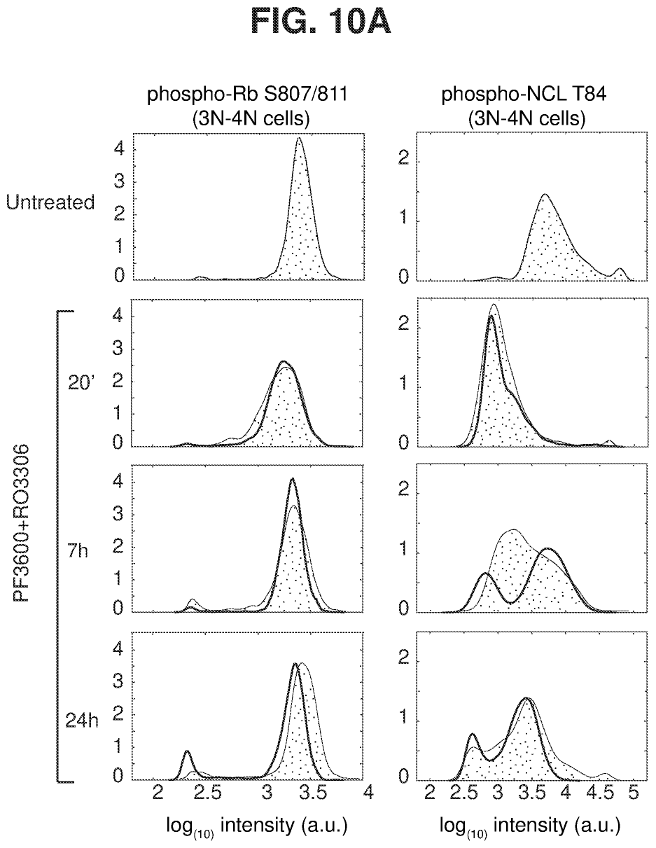

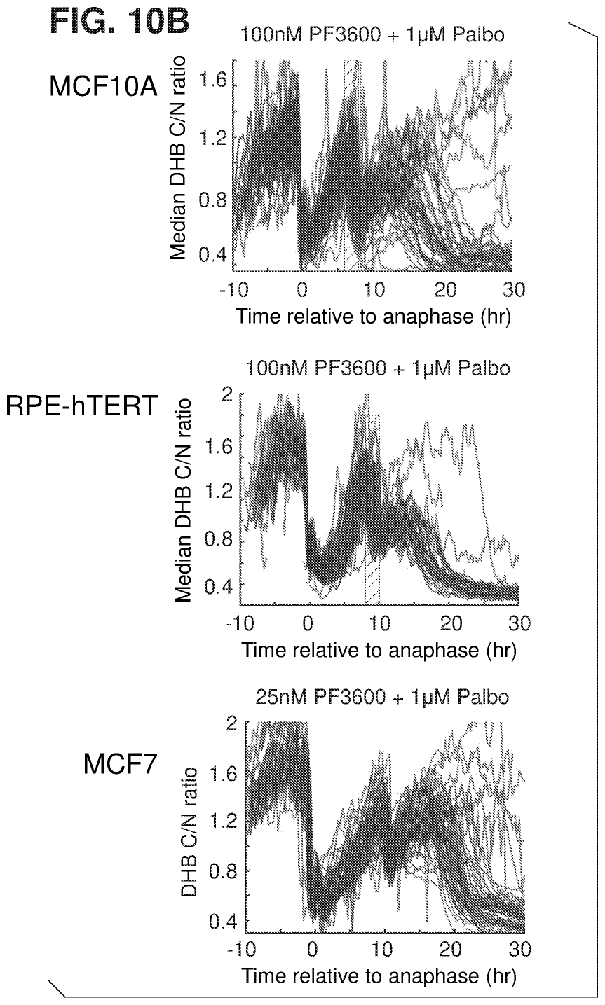

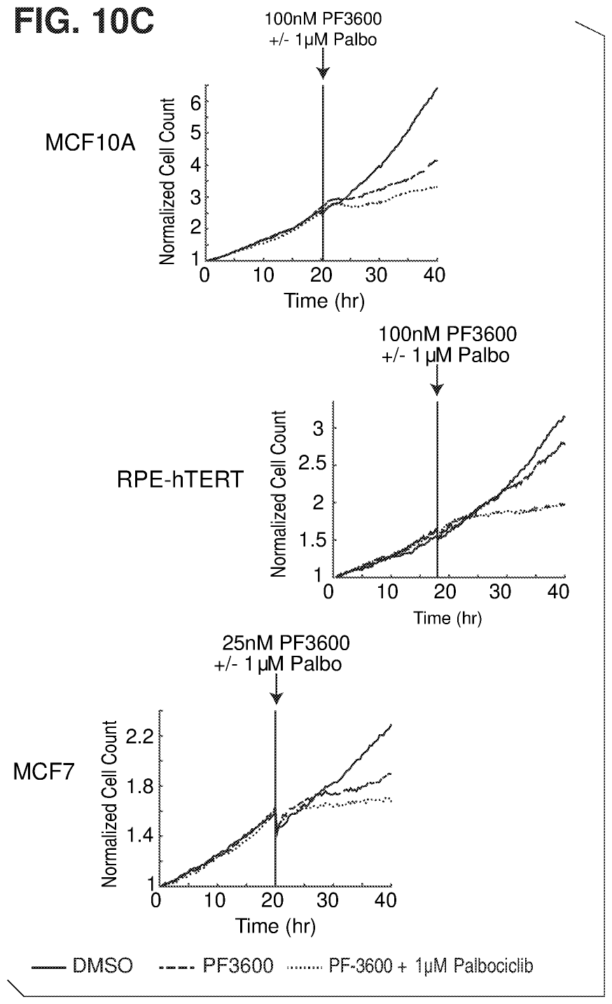

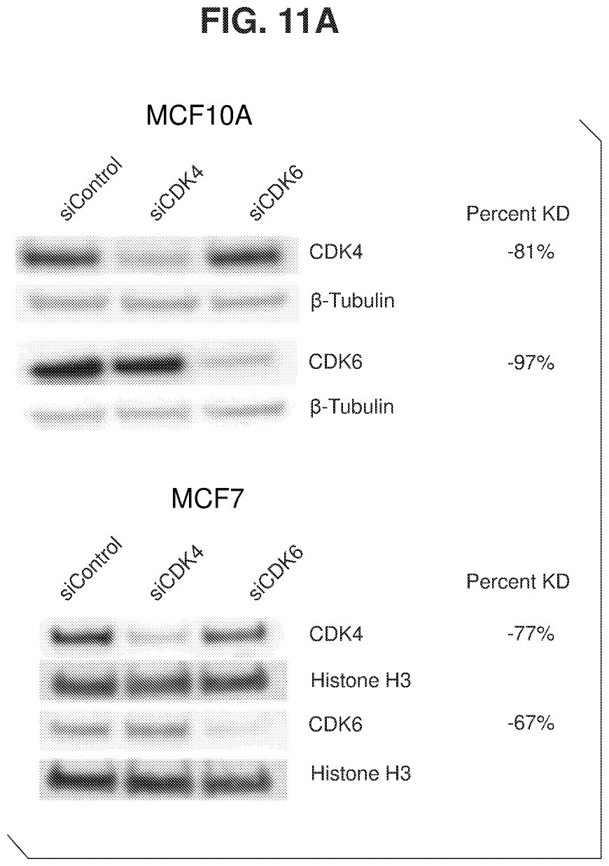

CDK1 has been shown to drive the complete cell cycle in CDK2/4/6 mouse knockouts by formation of non-canonical CDK1-Cyclin complexes (Aleem et al., 2005; Santamaria et al., 2007). Whether CDK1 could drive phosphorylation of CDK2 substrates upon acute CDK2 inhibition was investigated by time-lapse imaging in MCF10A, RPE-hTERT, and MCF7 cells by co-treating with RO3306, a CDK1 inhibitor (Vassilev et al., 2006). Contrary to expectation, co-inhibition of CDK2 (100 nM or 25 nM PF3600) and CDK1 (9 μM RO3306) still led to a rebound in phosphorylation of the DHB sensor (FIG. 5A), although the level of DHB phosphorylation achieved under these co-treatment conditions was somewhat lower. Similarly, co-inhibition of CDK2 (100 nM PF3600) and CDK1 (9 M RO3306) had no additional effect on Rb or Nucleolin phosphorylation as compared to CDK2-only inhibition (100 nM PF3600) (FIG. 10A). Together, these data suggest that upon inhibition of CDK2, DHB and other CDK2 substrates are only weakly phosphorylated by CDK1 in cells with fully functional CDK2. As co-inhibition of CDK2 and CDK1 did not abolish the rebound in substrate phosphorylation, the present inventors hypothesized the existence of alternate kinase(s) that may enable CDK2 substrate phosphorylation in the absence of CDK2 activity.

### Example 5: Investigation of Compensatory Kinase Activity: Activity was Abolished by CDK4/6 Inhibition

Previous studies in HCT116 colon cancer cells showed that when CDK2 was ablated though genetic approaches, Rb was still phosphorylated in cells. Elevated CDK4 activity was speculated to be the cause of this phosphorylation (Tetsu and McCormick, 2003), although this can be explained by the fact that Rb is also a CDK4/6 substrate. Similarly, although HCT116 cells are generally insensitive to palbociclib, genetic ablation of CDK2 renders them vulnerable to CDK4/6 inhibition. These observations imply that CDK4 could render CDK2 activity redundant in these cells, but phosphorylation of CDK2-specific substrates was not examined. Since the DHB sensor is not normally a CDK4/6 substrate (FIG. 3 and (Spencer et al., 2013)), the present inventors used the DHB sensor to investigate possible adaptation to PF3600 via compensation by CDK4/6/Cyclin D.

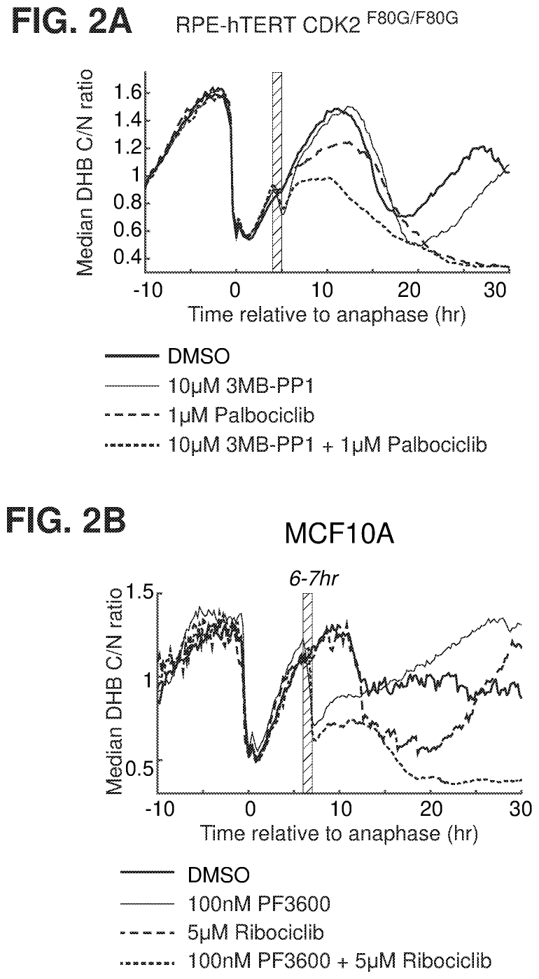

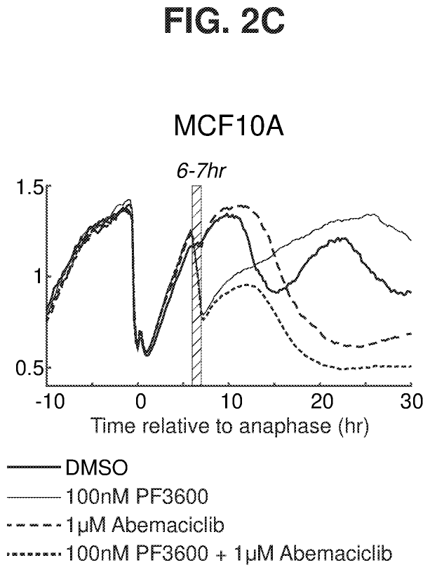

Co-inhibition of CDK4/6 and CDK2 with palbociclib (1 M) and PF3600 in MCF10A (100 nM PF3600), RPE-hTERT (100 nM PF3600) and MCF7 (25 nM PF3600) cells revealed a transient, rather than sustained, rebound in phosphorylation that subsequently fell to baseline levels for the remainder of the imaging period (FIG. 5B). These cells did not undergo any further mitoses suggesting that substrate phosphorylation critical to cell-cycle completion was blocked (FIGS. 10B and 10C). This phenomenon was not restricted to the DHB sensor as phosphorylation of Rb, Cdc6, and Nucleolin was rapidly lost after co-inhibition of CDK4/6 and CDK2, and no recovery was observed even 24 hr after treatment (FIGS. 5C and 5D). To test this effect in an orthogonal system, CDK2 and CDK4/6 were co-inhibited in RPE-hTERT CDK2^{F80G/F80G} cells with 10 μM 3 MB-PP1, 1 μM Palbociclib, or 10 μM 3 MB-PP1+1 μM Palbociclib at the indicated time. The sustained rebound phosphorylation previously seen with 3 MB-PP1 alone (FIG. 9A) was abolished upon co-treatment with 3 MB-PP1 and palbociclib (FIG. 2A). Number of single-cell traces: DMSO (133), 10 μM 3 MB-PP1 (104), 1 μM Palbociclib (146), 10 μM 3 MB-PP1+1 μM Palbociclib (160). DMSO and 10 μM 3 MB-PP1 median traces reproduced from FIG. 1D. These results support the notion that multiple CDK2 substrates are phosphorylated in a CDK4/6-dependent manner after acute inhibition of CDK2 activity. CDK2 and CDK4/6 were co-inhibited in MCF10A cells with 100 nM PF3600, 5 μM Ribociclib, 100 nM PF3600+5 μM Ribociclib (FIG. 2B). Number of single cell traces: DMSO (55), 100 nM PF3600 (53), 5 μM Ribociclib (23), 100 nM PF3600+5 μM Ribociclib (26) (FIG. 2B). CDK2 and CDK4/6 were co-inhibited in MCF10A cells with 100 nM PF3600, 1 μM Abemaciclib. or 100 nM PF3600+1 μM Abemaciclib (FIG. 2C). Number of single cell traces, right: DMSO (197), 100 nM PF3600 (242), 1 μM Abemaciclib (390), 100 nM PF3600+1 μM Abemaciclib (270) (FIG. 2C).

### Example 6: CDK4/6-Cyclin D Complexes Play a Crucial Role in the Rebound Phosphorylation of CDK2 Substrates

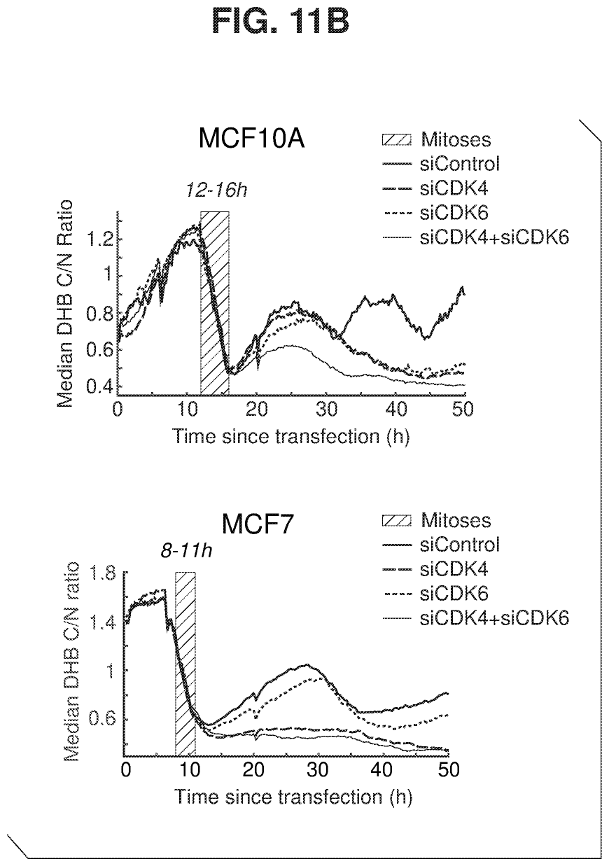

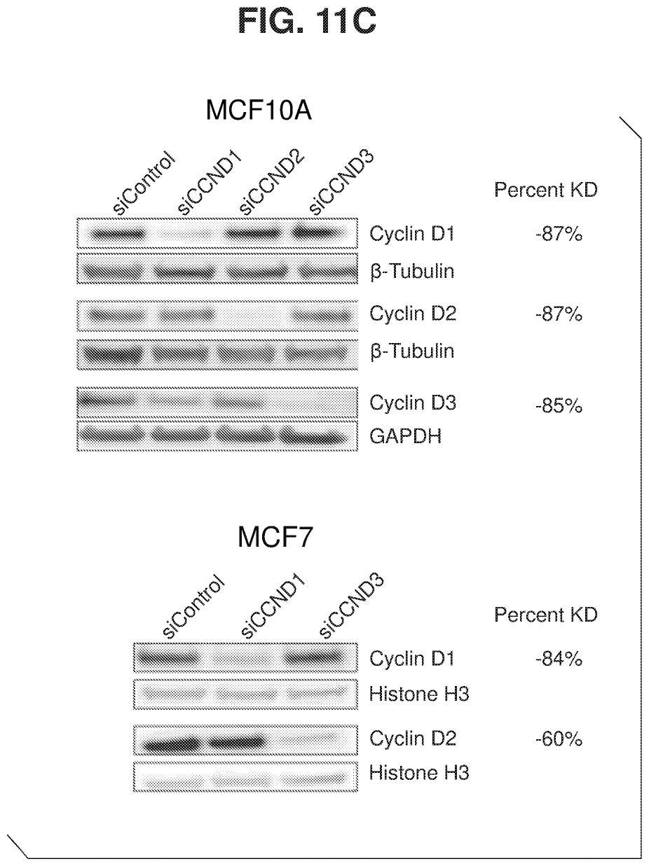

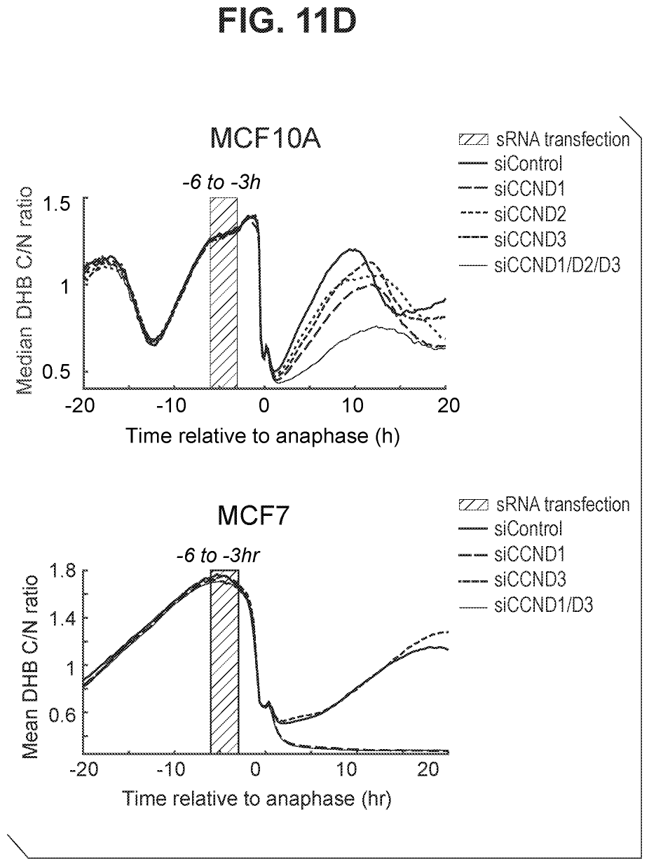

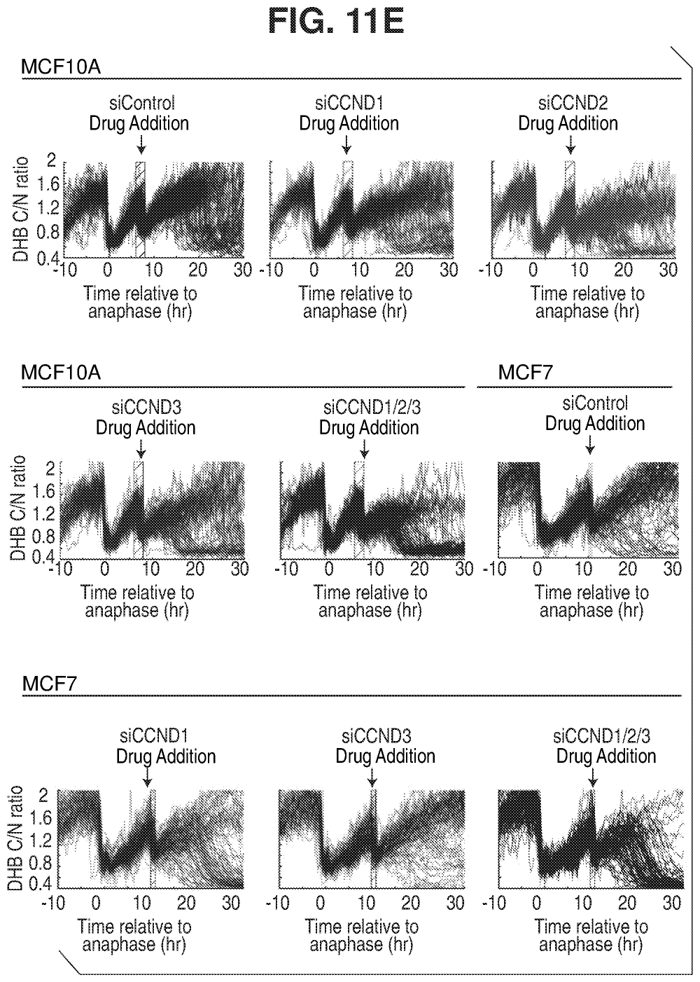

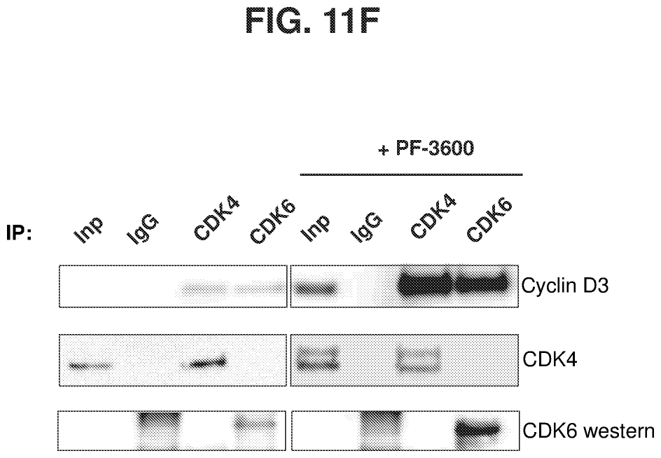

As CDK4 and CDK6 have both overlapping and unique cellular functions, determination of their individual contributions to the rebound in substrate phosphorylation seen after inhibition of CDK2 was of interest. siRNA-mediated knockdown of CDK4 or CDK6 in cycling MCF10A and MCF7 cells (FIG. 11A) revealed that MCF7 cells rely primarily on CDK4 for normal cell-cycle progression, whereas simultaneous knockdown of CDK4 and CDK6 was needed to block proliferation in MCF10A (FIG. 11B).

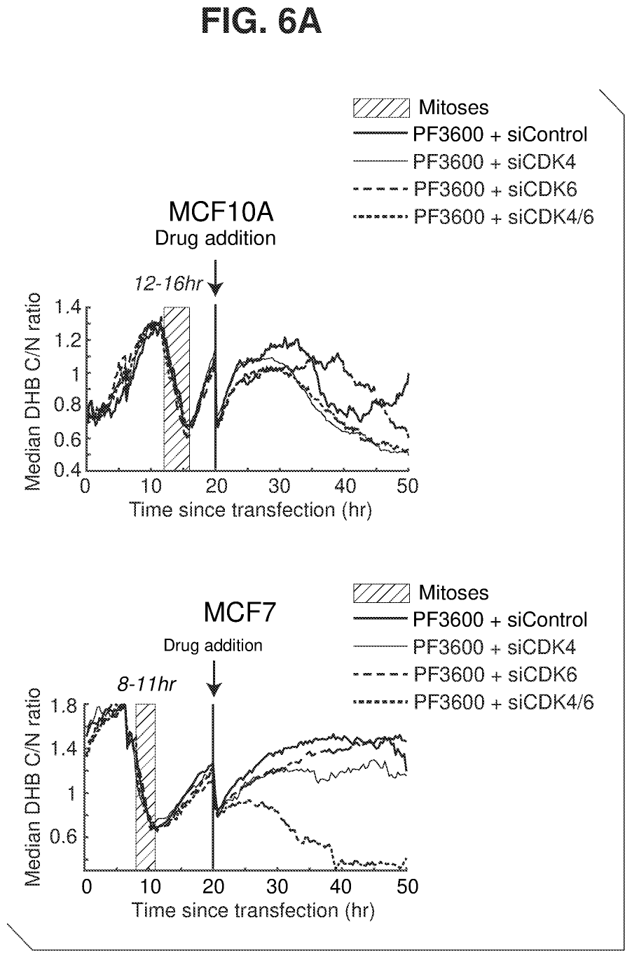

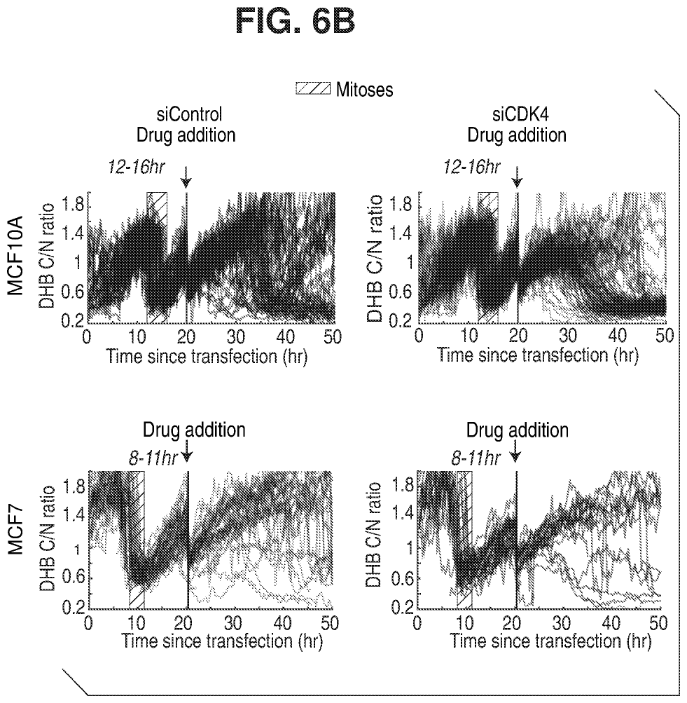

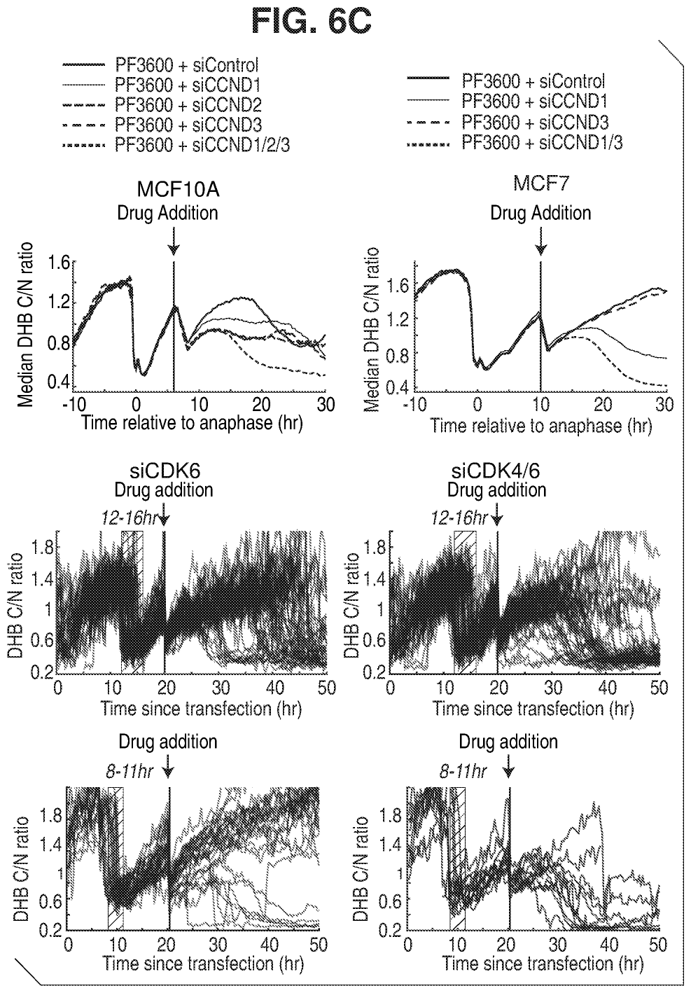

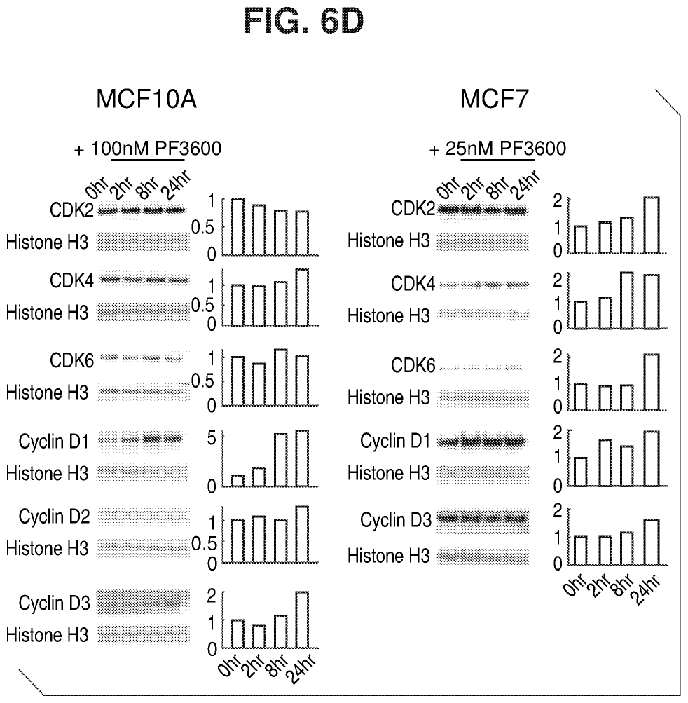

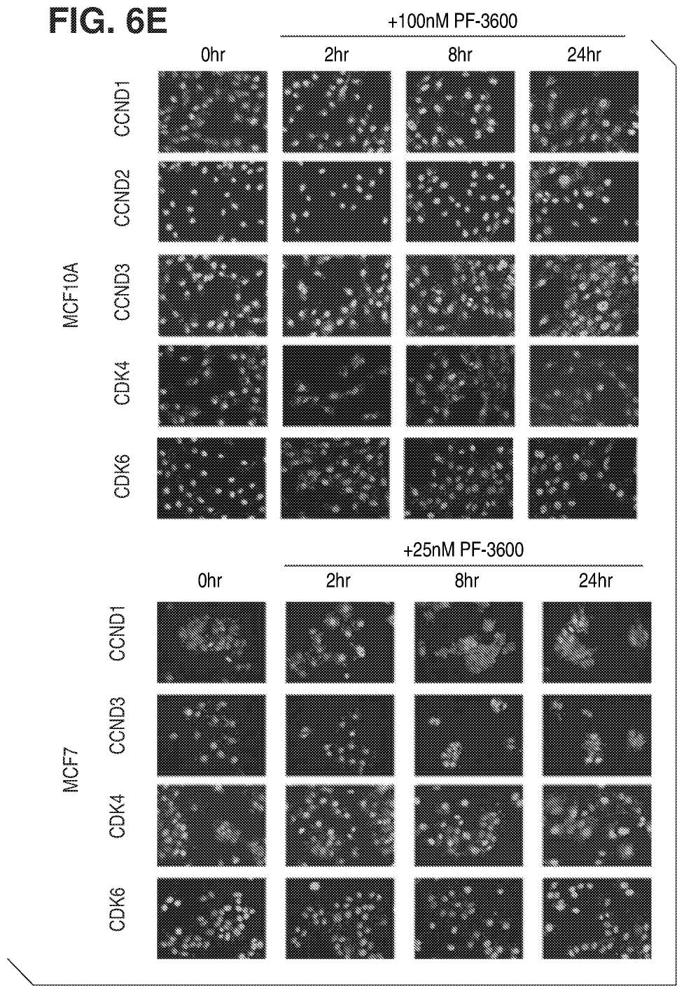

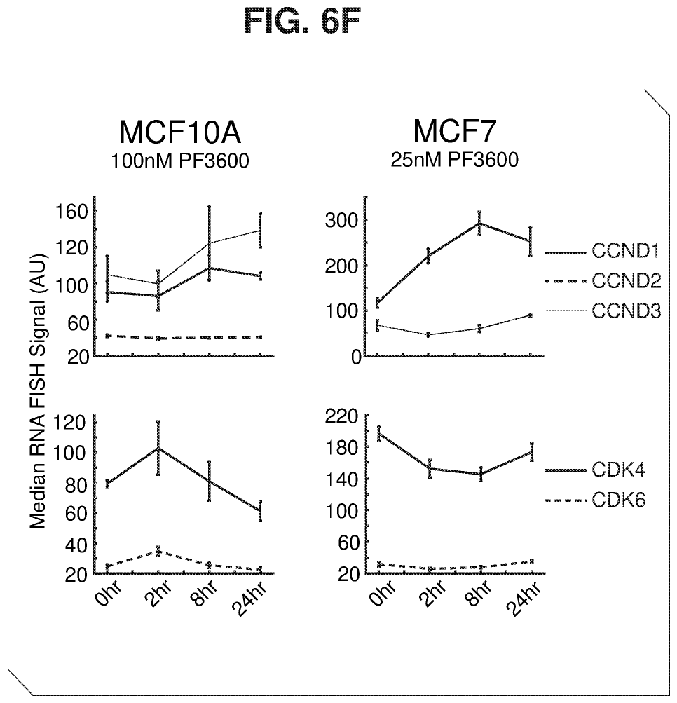

In PF3600-treated MCF10A cells, CDK4 knockdown was fairly effective at blocking the rebound phosphorylation of DHB (FIG. 6A, left), with a majority of the single-cell traces showing nuclear localization of the DHB sensor together with inhibition of mitosis (FIG. 6B, top). In contrast, in MCF7 cells, simultaneous knockdown of both CDK4 and CDK6 was needed to effectively block the rebound phosphorylation of DHB (FIGS. 6A, right and 6B, bottom). Thus, CDK4/6 knockdown phenocopies the observations made with PF3600 and palbociclib co-treatment (FIG. 5B).

Since CDK4 and CDK6 conventionally pair with D-type Cyclins, siRNA-mediated knockdown (FIG. 11C) was used to investigate which D-type Cyclins contribute to the compensatory kinase activity. In cycling MCF10A cells, all three Cyclins D1, D2, and D3 contributed to cell-cycle entry as only the triple knock-down had a strong effect on cell-cycle progression (FIG. 11D, top). As MCF7 cells do not express Cyclin D2 (Evron et al., 2001) the present inventors focused on Cyclin D1 and D3. In MCF7 cells, knockdown of Cyclin D1 effectively blocked cell-cycle progression, while Cyclin D3 was dispensable (FIG. 11D, bottom).

In MCF10A cells treated with PF3600, the rebound of DHB phosphorylation was partially blocked by knocking down Cyclin D1, D2, or D3 alone, whereas triple knockdown of the D-type cyclins abrogated the sustained rebound (FIGS. 6C, left and 11E, top). In MCF7 cells treated with PF3600, knocking down Cyclin D3 had minimal effect, whereas targeting Cyclin D1 largely blocked the rebound of DHB phosphorylation, and simultaneous knockdown of Cyclins D1 and D3 was even more effective at preventing this rebound (FIG. 6C, right and FIG. 11E, bottom). Thus, knocking down Cyclins D1, D2, and D3 (MCF10A) or Cyclin D1 (MCF7) phenocopies the observations made with PF3600 and palbociclib co-treatment shown in FIG. 5B. In summary, MCF10A cells can use CDK4, CDK6, Cyclin D1, Cyclin D2, and Cyclin D3 for normal cell-cycle entry but rely slightly more on CDK4/Cyclin D2 and D3 for the rebound phosphorylation upon acute CDK2 inhibition. In contrast, MCF7 cells use CDK4/Cyclin D1 for normal cell-cycle progression but rely on both CDK4 and CDK6 along with Cyclin D1 for the rebound phosphorylation upon acute CDK2 inhibition.

### Example 7: Cyclin D1 and D3 Levels were Upregulated Upon CDK2 Inhibition

The mechanisms driving the rebound kinase activity post CDK2 inhibition were investigated. In MCF10A cells, an increase in Cyclin D1 and D3 protein levels was observed within 24 hr PF3600 (100 nM) treatment while CDK2, CDK4 and CDK6 protein levels remained stable (FIG. 6D). In contrast, in MCF7 cells, Cyclin D1, Cyclin D3, CDK2, CDK4 and CDK6 protein levels all increased to varying degrees within 24 hr PF3600 (25 nM) treatment. To determine if the upregulation occurred at the level of transcription, mRNA FISH was performed to measure the expression of CDK4, CDK6, and Cyclins D1, D2 and D3. In agreement with the western blot results, an increase in mRNA expression of Cyclins D1 and D3 was observed in MCF10A, and a particularly strong increase in Cyclin D1 was observed in MCF7 cells, in response to PF3600 treatment (FIG. 6E-F). These findings at least partially explain the increased protein levels.

To determine if the upregulated Cyclin D protein levels translated to increased CDK-Cyclin D3 complexes in MCF10A cells, immunoprecipitation of CDK4 and CDK6 was performed in MCF10A cells treated for 24 hr with either DMSO or PF3600 and probed for Cyclin D3. Indeed, higher amounts of Cyclin D3 bound to both CDK4 and CDK6 was observed after CDK2 inhibition (FIG. 11F). Together, these data substantiate that acute inhibition of CDK2 triggered upregulation of cell-type specific CDK4/6/Cyclin D complexes, a subset of which may promote rebound phosphorylation of CDK2 substrates.

### Example 8: CDK4/6 and CDK2 Compensate for One Another In Vivo

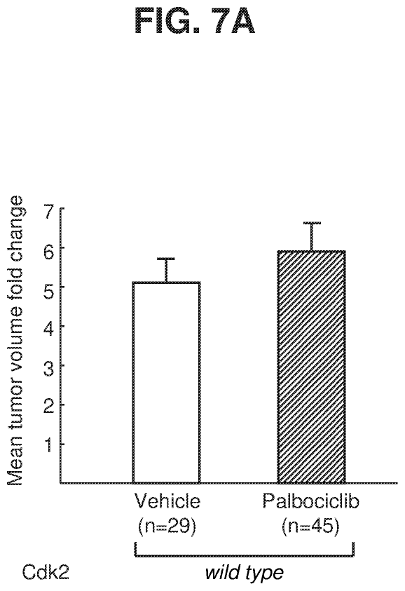

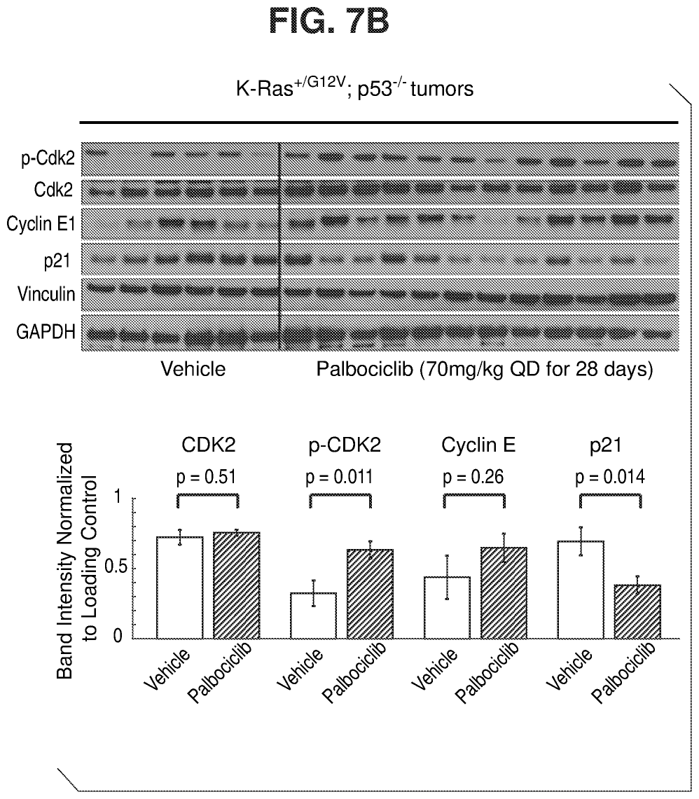

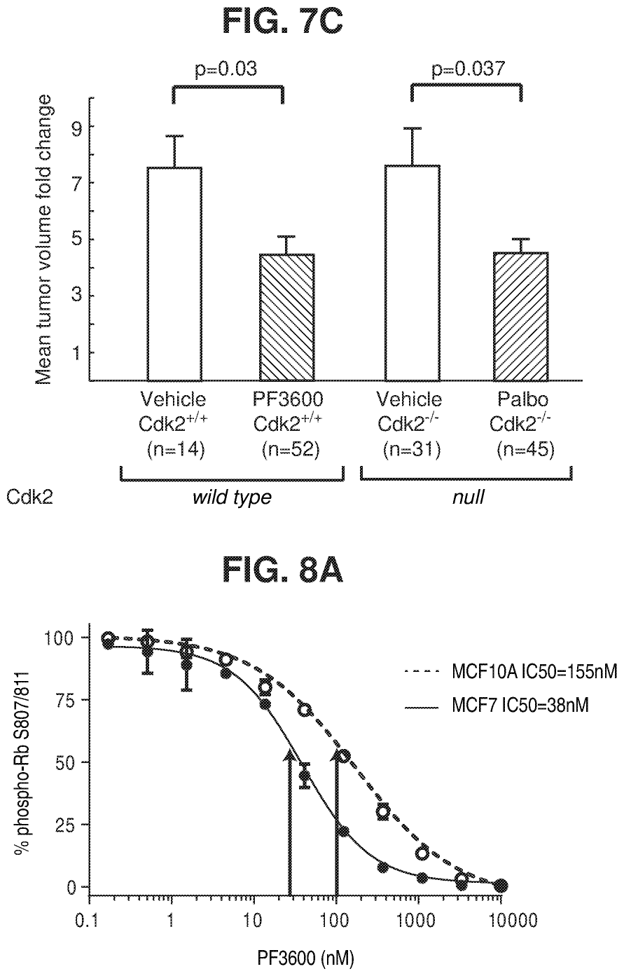

To test the potential compensatory relationship between CDK2 and CDK4/6 in vivo, an established mouse lung tumor model driven by KRAS^{G12V} and TRP53 mutations was used. Kras^{+/LSLG12V}, Trp53^{L/L} mice intra-nasally infected with adenoviral particles encoding Cre recombinase developed lung tumors with a latency of 5 months. Once tumor development was detected by CT scans, animals were treated with either vehicle or palbociclib (70 mg/kg QD) for 28 days and tumor volumes were subsequently measured by CT scans at the end of the treatment period. A comparison of tumor volume fold changes between vehicle- and palbociclib-treated mice showed no significant reduction in tumor burden after palbociclib treatment (FIG. 7A). However, upregulation of CDK2 T-loop phosphorylation and Cyclin E1 was detected by western blot in the palbociclib-treated lung tumors. Together with significant downregulation of the CDK inhibitor p21 (FIG. 7B), these data indicate that upregulation of CDK2 activity could explain the insensitivity to palbociclib.

To test whether CDK2 plays a role in the palbociclib-resistant tumors, Kras^{+/LSLG12V}; Trp53^{L/L}; Cdk2^{-/-} mice were generated. These mice developed lung tumors of similar size as the Kras^{+/LSLG12V}; Trp53^{L/L}; Cdk2^{+/+} control animals (FIG. 7C). Remarkably, palbociclib treatment of Cdk2-null lung adenocarcinoma-bearing mice led to significantly reduced tumor size (p=0.037) (FIG. 7C). Thus, in this tumor setting, CDK4/6 inhibition was sufficient to suppress tumor growth in the absence of CDK2.

To further support the idea that CDK2 and CDK4/6 both contribute to tumor growth, CDK2, CDK4, and CDK6 activity in Kras^{+/LSLG12V}; Trp53^{L/L} lung tumor-bearing mice was inhibited by treating with PF3600 at 50 mg/kg BID (a higher dose than used in cellular studies presented thus far, covering CDK2, CDK4, and CDK6.) Consistent with the palbociclib sensitivity of the Cdk2-null tumors, inhibition of CDK2/4/6 with PF3600 led to significantly reduced tumor volumes (FIG. 7C). Taken together, the in vivo data support the hypothesis that CDK2 and CDK4/6 kinases can perform overlapping functions and compensate for one another.

### Example 9: Materials and Methods

### Experimental Model Details:

Cell lines used in this study were obtained from ATCC with the exception of the RPE-hTERT wild-type and CDK2 analog sensitive cells which were courtesy of Robert Fisher lab at Icahn School of Medicine. MCF10A (human breast epithelial) cells were cultured in DMEM/F12 supplemented with 5% horse serum (Invitrogen), 20 ng/mL epidermal growth factor (Sigma-Aldrich), 0.5 mg/mL hydrocortisone (Sigma-Aldrich), 100 ng/mL cholera toxin (Sigma-Aldrich), and 10 μg/mL insulin (Invitrogen). RPE-hTERT cells were grown in DMEM with 10% FBS. MCF7 and OVCAR3 cells were grown using RPMI-1640 supplemented with 10% FBS. Except during siRNA transfections, all full-growth media were supplemented with Penicillin/Streptomycin. All cells were cultured at 37° C. with 5% CO₂.

### Stable Cell Line Generation Using Lentiviral Vectors:

Cells stably expressing the CDK2 sensor (DHB-mVenus or DHB-mCherry), and H2B tagged with mTurquoise were generated by lentivirus transduction. For virus generation, HEK293T cells were transfected with CSII-EF plasmid (CSII-EF DHB-mVenus, CSII-EF DHB-mCherry, CSII-EF CDC6-YFP, or CSII-EF H2B-mTurquoise) along with the helper packaging and envelope plasmids (pMDLg, pRSV-Rev, pCMV-VSV-G) using the Fugene-HD reagent (Promega E2311). Lentivirus was harvested 48 hr after transfection, filtered through a 0.45 μm filter (Millipore), and incubated with target cells for 6-10 hr in presence of 5 μg/ml polybrene (EMD Millipore #TR-1003). Cells with stable integrations were sorted on an Aria Fusion Flow Cytometer to establish a population where all cells express the desired sensors. siRNA transfections

siRNA transfections were carried out with Dharmafect1 reagent (Dharmacon) using the manufacturer's protocol. For each target, a pool of siRNAs targeting three different regions of the gene were used. Cells were incubated for 6-7 hr with the transfection complexes in full-growth media lacking antibiotics. The siRNA sequences used in the study were as follows: CCND1 (Dharmacon #MU-003210-05-0002), CCND2 (Dharmacon #MU-003210-05-0002), CCND3 (Dharmacon #J-003212-10-0002, J-003212-11-0002, J-003212-12-0002), CDK4 (IDT Product #198569326, 198569329, 198569332), CDK6 (IDT Product #200925870, 200925873, 200925876).

### Time-Lapse Imaging:

Cells were seeded at least 24 hr prior to imaging in phenol-red-free full-growth medium in glass-bottom 96-well plates (CellVis P96-1.5H-N) that were coated with collagen prior to seeding. The seeding density was chosen such that the cells would remain sub-confluent until the end of the imaging period. Cells were first imaged for 16-20 hr in full-growth media without drug. The movie was then briefly paused and the full-growth media was replaced with full-growth media containing drug at the desired concentration. The plate was then re-inserted into the microscope and aligned to its prior position and imaging was continued for an additional 24-48 hr. Images were acquired every 12 min (for MCF10A or RPE-hTERT) or every 20 min (for MCF7 or OVCAR3) on a Nikon Eclipse Ti or Ti2 microscope with a 10×0.45 NA objective in a humidified, 37° C. chamber at 5% CO2. Exposure times for all movies for all channels were kept under 500 ms per timepoint to minimize phototoxicity. Cell tracking was performed using published MATLAB scripts (Cappell et al., 2016), as described previously (Arora et al., 2017). The tracking code is available for download at https://github.com/scappell/Cell_tracking. Cell counts over time were obtained by counting the number of segmented nuclei in each frame of the movie.

### Immunofluorescence:

Cells were fixed for 15 minutes with freshly prepared 4% paraformaldehyde, washed twice with PBS, and incubated with a blocking buffer (3% BSA in PBS) for 1 hr at room temperature. Permeabilization was carried out using 0.2% Triton-X 100 for 15 min at 4° C. Primary antibody was diluted in blocking buffer and incubated with cells overnight at 4° C. followed by three washes with 1×PBS. Secondary antibodies conjugated to Alexa Fluor 488, Alexa Fluor 546 or Alexa Fluor 647 were incubated for 1 hr followed by three washes with 1×PBS. DNA was stained using Hoechst 33342 dye for 10 minutes at 1:10000 (ThermoFisher H3570). Images were acquired on a Nikon Eclipse Ti or Ti2 microscope with a 10×0.45 NA objective. DNA content was determined by taking the integrated intensity of each cell's Hoechst signal. Cells were delineated as 3-4N DNA content by plotting a histogram of DNA content and drawing a threshold and the end of the 2N peak.

### Antibodies and Reagents:

Antibodies used in this study were phospho-Rb (Ser807/811) (CST 8516), phospho-Nucleolin (Thr84) (Abcam ab196338), phospho-NBS1 (Ser432) (Abcam ab12297), phospho-CDC6 (Ser54) (Abcam ab75809), GAPDH (CST 5174 in FIG. 4, Invitrogen ZG003 in FIG. 5), β-tubulin (CST 86298), Histone H3 (CST), CDK2 (Abcam ab32147), CDK4 (Abcam ab108357), CDK6 (Abcam ab151247 and ab124821), Cyclin D1 (Cyclin D1 clone SP4 (Thermo Scientific RM-9140-S0), Cyclin D2 (CST 3741), Cyclin D3 (CST 2936), phospho-CDK2 T160 (Cell Signaling 2561), CDK2 (Abcam 32147), p²¹ (Santa Cruz 6246), Cyclin E (Santa Cruz 481), Vinculin (Sigma V9131), Alexa 488 goat anti-mouse (Thermo Fisher Scientific, A-11001), Alexa Fluor 546 goat anti-rabbit (Thermo Fisher Scientific, A-11035) and Alexa Fluor 647 goat anti-rabbit (Thermo Fisher Scientific, A-21245).

Palbociclib and PF3600 were dissolved in anhydrous DMSO (Sigma-Aldrich Cat. No.: 276855); Palbociclib was added to a final concentration of 1 μM and P3600 was added to a final concentration of 25 nM, 100 nM, or 500 nM as indicated. Abemaciclib (Cat. No.: HY-16297A) and Ribociclib (Cat. No.: HY-15777) were purchased from MedChemExpress and dissolved in anhydrous DMSO. Abemaciclib was added to a final concentration of 1 μM and Ribociclib was added to a final concentration of 5 M. 3 MB-PP1 (Cayman Chemical Cat. No.: 17860) was dissolved in anhydrous DMSO and was added to a final concentration of 10 μM. RO3306 (#SML0569) was purchased from Sigma Aldrich.

### Co-Immunoprecipitations:

MCF10A cells treated with DMSO or 100 nM PF3600 for 24 hr were lysed using 1×cell lysis buffer (CST 9803) supplemented with phenylmethylsulfonyl fluoride (PMSF), phosphatase inhibitors, and protease inhibitors (1:1000 dilution of Sigma-Aldrich P8340). Total protein concentration in the lysates was measured using a Bradford Assay and equal quantities of protein were incubated with 5 μg of antibody overnight at 4° C. The antigen-antibody complexes were pulled down by incubating with Protein G Dynabeads (ThermoFisher 10003D) and washed three times with 1×lysis buffer. Proteins bound to the beads were eluted using 1×LDS sample buffer (ThermoFisher NP0007) and analyzed by western blotting.

### Phospho-Serine 807/811 Rb ELISA:

MCF10A or MCF7 cells were seeded at 25,000 cells/well in growth media in 96-well cell culture plates and allowed to adhere at 37° C. with 5% CO₂ overnight. The following day, compounds were serially diluted from 10 mM stock for 11-point 3-fold dilution curve in DMSO (Sigma). Compounds were intermediately diluted 1:200 into growth media prior to diluting 1:5 on cells for final concentration 10 μM top dose in 0.1% DMSO on cells. Cells were treated for 1 hr at 37° C. in 5% CO₂. Cells were lysed in lysis buffer (Cell Signaling Technologies, 9803) containing protease inhibitor cocktail (Cell Signaling Technologies, 5872), SDS, and PMSF on ice and transferred to pre-coated and blocked anti-phospho-Ser807/811 Rb (Cell Signaling Technologies, 8516) ELISA plates for overnight incubation at 4° C. Plates were washed with phosphate buffered saline to remove residual unbound cellular proteins and total Rb detection antibody (Cell Signaling Technologies, 9309) was added for 90 min at 37° C. Following washing to remove unbound total Rb antibody, HRP-tagged antibody (Cell Signaling Technologies, 7076) was allowed to bind for 30 min at 37° C. Following washing to remove unbound HRP antibody, Glo Substrate Reagent (R&D Systems, DY993) was added and incubated protected from light for 5-10 min. Plates were read in luminescent mode on an Envision plate reader (Perkin Elmer) and IC50 values calculated using GraphPad Prism Version 8.0.2

### Western Blotting:

Lysates were prepared using 1×LDS sample buffer (ThermoFisher NP007) using equal numbers of cells. Proteins were separated using NuPAGE precast polyacrylamide gels (ThermoFisher NP0301). Total protein was quantified using Azure Red Dye (Azure Biosystems AC2124) and was used to normalize the signal from antibodies of interest. Primary antibodies used are specified under the “Antibodies” section. HRP-conjugated or IR700 and IR800 labeled fluorescent secondary antibodies were used for visualization (Cell Signaling Technology 7074 and 7076).

For western blots in FIG. 7B, protein extraction was performed in protein lysis buffer (50 mM Tris-HCl pH 7.4, 150 mM NaCl, 0.5% NP-40) supplemented with a cocktail of protease and phosphatase inhibitors (complete Mini, Roche, 11836153001; Phosphatase Inhibitor Cocktail 2 and 3, Sigma, P5726 and P0044). Protein concentrations were measured using Bradford (Bio-Rad) method. 25 g of protein extracts obtained from tumor tissue were separated on NUPAGE™ 4-12% Bis-Tris Midi gels (Invitrogen), transferred to a nitrocellulose membrane (GE Healthcare) and blotted with primary antibodies. Primary antibodies were detected with goat secondary antibodies directed against mouse or rabbit IgGs (HRP, Dako, and Alexa Fluor 680, Invitrogen) and visualized with ECL Western Blot detection solution (GE Healthcare).

mRNA FISH:

Target-specific mRNA probes were obtained from ThermoFisher, and ViewRNA ISH kit (QVC001) together with the manufacturer's protocol were used to detect target mRNA expression in single cells. Probes used in the study are CCND1 (VA6-16943-VC), CCND2 (VA4-3083615-VC), CCND3 (VA6-17696-VC), CDK4 (VA6-18880) and CDK6 (VA6-3169253). For quantification of the mRNA FISH signal, Hoechst was used to obtain a nuclear mask, which was dilated by 1 pixel in order to obtain the median cytoplasmic signal intensity per cell.

### Single-Cell CDK2 Activity Analysis:

Asynchronously cycling cells that divided and received drug treatments during the imaging period were initially segmented into categories based on the cells' time of anaphase relative to the time of drug addition. These cells were then additionally subcategorized based on their DHB cytoplasmic/nuclear (C/N) ratio after the mitotic event. C/N ratio was calculated by quantifying the ratio of cytoplasmic to nuclear mean DHB fluorescence, with the cytoplasmic component calculated as the mean of the top 50th percentile of a ring of pixels outside of the nuclear mask.

Cells were classified as CDK2^{inc} if the DHB C/N ratio was above 0.5 units 3 hr after anaphase, otherwise they were classified as CDK2^{low}. Figure legends indicate whether only CDK2^{inc} cells or whether all cells are plotted. The median of the single-cell traces in a subcategory was then used to create median trace with 95% confidence interval representative of the particular subcategory. All cell trace analysis was done using custom MATLAB scripts and code is available upon request.

### Phospho-Proteomics:

Phospho-peptide enrichment was performed as previously described (Lapek et al., 2017b). Lyophilized phospho-peptides were then TMT-labeled and fractionated by reverse phase basic pH fractionation and fractions combined as previously described (Edwards and Haas, 2016). Fractions were lyophilized and stored at 80° C. until MS analysis. For LC-MS2/MS3 analysis, samples were reconstituted in 10 μL of 5% acetonitrile in 5% formic acid and 8 μL of each fraction was injected on the Orbitrap Fusion Lumos for analysis.

Peptides were eluted on a 165-minute gradient of 7-32% solvent B (80% acetonitrile in 0.1% formic acid) at 300 nL/min on a PepMap RSLC C18 column (2 μm, 100 Å, 75 μm×50 cm) heated to 60° C. Spectra were acquired in Top Speed mode, with a 5 second cycle. MS1 data were collected in the Orbitrap at 60000 resolution across a range of 500-1500 m/z. An automatic gain control (AGC) target of 2×10⁵ was used with a maximum injection time of 100 ms. MS2 data were acquired in the ion trap with a rapid scan rate, maximum injection time of 70 ms, and an AGC target of 2×10⁴. The quadrupole was used for isolation, with an isolation window of 0.5 m/z. Peptides were fragmented with CID at 30% normalized collision energy with an activation time of 10 ms and an activation Q of 0.25. For MS3 spectra, up to 10 ions were selected for synchronous precursor selection, and data were collected at 60000 resolution in the Orbitrap. Ions were fragmented with HCD at an energy of 55%. MS3 AGC was set to 1×10⁵ with a maximum injection time of 250 ms and a first mass of 110 m/z. Data at all stages were centroided.

Resultant raw files were processed on an IP2GPU server (Integrated Proteomics Applications, Inc.). Data were searched with the ProLuCID algorithm (Xu et al., 2015) against the Uniprot Human Database (Downloaded Jan. 29, 2018) concatenated with the current contaminants database and reverse database. Carbamidomethylation of Cysteine residues (+57.02146) and TMT-11 modification of peptide n-termini and Lysine residues (+229.162932) were included as static modifications. Oxidation of Methionine (+15.9949) and phosphorylation of Serine, Threonine, and Tyrosine (+79.966331) were included as variable modifications. A maximum of 4 variable modifications and two missed cleavages were allowed. Peptides had to have a minimum length of 6 amino acids to be considered. Data were searched with a 50 ppm MS1 tolerance (Huttlin et al., 2010) and 800 ppm MS2 tolerance. Final data were filtered to a 1% protein level false discovery rate.

Data were normalized in a multistep process as previously described (Lapek et al., 2017a). Briefly, first data were normalized to a pooled bridge channel to account for run-to-run instrument performance differences and then median scrubbed to account for any mixing errors. All phospho data was processed and analyzed at the peptide level.

### Experimental Model Details:

Cell lines used in this study were obtained from ATCC with the exception of the RPE-hTERT wild-type and CDK2 analog sensitive cells which were courtesy of Robert Fisher lab at Icahn School of Medicine. MCF10A (human breast epithelial) cells were cultured in DMEM/F12 supplemented with 5% horse serum (Invitrogen), 20 ng/mL epidermal growth factor (Sigma-Aldrich), 0.5 mg/mL hydrocortisone (Sigma-Aldrich), 100 ng/mL cholera toxin (Sigma-Aldrich), and 10 μg/mL insulin (Invitrogen). RPE-hTERT cells were grown in DMEM with 10% FBS. MCF7 and OVCAR3 cells were grown using RPMI-1640 supplemented with 10% FBS. Except during siRNA transfections, all full-growth media were supplemented with Penicillin/Streptomycin. All cells were cultured at 37° C. with 5% CO2.

### Mouse Studies:

Mice: Kras^{+/LSLG12V} Trp53^{L/L} and Cdk2^{-/-} mice have been previously described (Guerra et al., 2003; Jonkers et al., 2001; Ortega et al., 2003). Compound mice using the following transgenes: Kras^{+/LSLG12V}, (Guerra et al., 2003); Trp53^{L/L} (Lee et al., 2012), and Cdk2^{-/-} (Ortega et al., 2003) were generated for this study. All animal experiments were approved by the Ethical Committees of the Spanish National Cancer Research Centre (CNIO), the Carlos III Health Institute and the Autonomous Community of Madrid (PROEX 270/14) and were performed in accordance with the guidelines stated in the International Guiding Principles for Biomedical Research Involving Animals, developed by the Council for International Organizations of Medical Sciences (CIOMS). Mice were housed in specific-pathogen-free conditions at CNIO's Animal Facility (AAALAC, JRS:dpR 001659). Female and male mice were used for the experiments. All mice were genotyped at the CNIO's Genomic Unit.

Lung tumor induction: Induction of lung adenocarcinomas was carried out in anesthetized (ketamine 75 mg/kg, xylazine 12 mg/kg) 8-week old mice by intra-nasal instillation of a single dose of 10⁶ pfu/mouse of adeno viruses encoding the Cre recombinase (Ad-Cre). All the adenoviral preparations were purchased from the University of Iowa (Iowa City, USA).

Micro CT imaging: Image studies were done by the Molecular Imaging Core Unit at the CNIO. Mice were anesthetized with a continuous flow of 1% to 3% isoflurane/oxygen mixture (0.5 L/min) and the chest area was imaged by three-dimensional microcomputed tomography performed with a CompaCT scanner (SEDECAL Madrid SpainGE). Data were acquired with 720 projections by 360-degree scan, integration time of 100 ms with three frames, photon energy of 50 KeV, and current of 100 uA. Tumor measurements were obtained with GE MicroView software v2.2. Tumor volume was calculated as follows: (short axis x short axis x long axis/2).

Pharmacological treatment in mice: Kras^{+/LSLG12V}; Trp53^{L/L}; Cdk2^{-/-} and Kras^{+/LSLG12V}; Trp53^{L/L}; Cdk2^{+/+} mice were infected with 10⁶ pfu of Ad-Cre. Once tumors were detected by CT, mice harboring at least one tumor bigger than 3 mm³ were enrolled in the different treatment groups. Palbociclib was dosed at 70 mg/kg QD for 4 weeks and PF3600 was dosed at 50 mg/kg BID for 4 weeks. Drug efficacy was monitored by CT measurements.

TABLE 1
Target Protein Phosphorylation Levels at 1 hr and
24 hr post treatment (25 nM PF3600)

| | | | | 3600_1 | 3600_24 |
|---|---|---|---|---|---|
| Protein | Description | Gene | SEQUENCE | hr_log2 | hr_log2 |
| E7EVAO | Microtubule- | MAP4 | DVPPLSETEApTPVPIK | −2.05 | 1.30 |
| | associated | | (SEQ ID NO: 1) | | |
| | protein | | | | |
| | OS = Homo | | | | |
| | sapiens | | | | |
| | GN = MAP4 | | | | |
| | PE = 1 SV = 1 | | | | |
| P12270 | Nucleoprotein | TPR | GIASTSDPPTANIKPTP | −2.33 | 0.16 |
| | TPR OS = Homo | | VVSpTPSK | | |
| | sapiens | | (SEQ ID NO: 2) | | |
| | GN = TPR PE = 1 | | | | |
| | SV = 3 | | | | |
| Q13330 | Metastasis- | MTAI | SVSSVLSSLpTPAK | −2.82 | −0.89 |
| | associated | | (SEQ ID NO: 3) | | |
| | protein MTAI | | | | |
| | OS = Homo | | | | |
| | sapiens | | | | |
| | GN = MTA1 | | | | |
| | PE = 1 SV = 2 | | | | |
| Q9UBF8 | Phosphatidylinositol | PI4KB | ELPSLSPAPDTGLpSPS | −2.50 | 0.71 |
| | 4-kinase beta | | K | | |
| | OS = Homo | | (SEQ ID NO: 4) | | |
| | sapiens | | | | |
| | GN = PI4KB | | | | |
| | PE = 1 SV = 1 | | | | |
| Q13330 | Metastasis- | MTAI | AGVVNGTGAPGQpSPG | −0.98 | −0.63 |
| | associated | | AGR | | |
| | protein MTAI | | (SEQ ID NO: 5) | | |
| | OS = Homo | | | | |
| | sapiens | | | | |
| | GN = MTA1 | | | | |
| | PE = 1 SV = 2 | | | | |
| P31689 | DnaJ homolog | DNAJA1 | VNFPENGFLpSPDK | −1.15 | 0.01 |
| | subfamily A | | (SEQ ID NO: 6) | | |
| | member 1 | | | | |
| | OS = Homo | | | | |
| | sapiens | | | | |
| | GN = DNAJA1 | | | | |
| | PE = 1 SV = 2 | | | | |
| P28749 | Retinoblastoma- | RBL1 | RVIAIDSDAEpSPAK | −1.39 | −0.40 |
| | like protein 1 | | (SEQ ID NO: 7) | | |
| | OS = Homo | | | | |
| | sapiens | | | | |
| | GN = RBL1PE = 1 | | | | |
| | SV = 3 | | | | |
| Q8WYP5-3 | Isoform 3 of | AHCTF1 | GLSQNQQIPQNSVpTPR | −2.29 | 0.54 |
| | Protein ELYS | | (SEQ ID NO: 8) | | |
| | OS = Homo | | | | |
| | sapiens | | | | |
| | GN = AHCTF1 | | | | |
| Q09666 | Neuroblast | AHNAK | ISMQDVDLSLGpSPK | −1.85 | 0.14 |
| | differentiation- | | (SEQ ID NO: 9) | | |
| | associated | | | | |
| | protein AHNAK | | | | |
| | OS = Homo | | | | |
| | sapiens | | | | |
| | GN = AHNAK | | | | |
| | PE = 1 SV = 2 | | | | |
| Q09666 | Neuroblast | AHNAK | GGVTGSPEASISGpSK | −2.39 | −0.92 |
| | differentiation- | | (SEQ ID NO: 10) | | |
| | associated | | | | |
| | protein AHNAK | | | | |
| | OS = Homo | | | | |
| | sapiens | | | | |
| | GN = AHNAK | | | | |
| | PE = 1 SV = 2 | | | | |
| Q14004 | Cyclin- | CDK13 | TNpTPQGVLPSSQLK | −1.72 | 0.00 |
| | dependent kinase | | (SEQ ID NO: 11) | | |
| | 13 OS = Homo | | | | |
| | sapiens | | | | |
| | GN = CDK13 | | | | |
| | PE = 1 SV = 2 | | | | |
| Q53GA4 | Pleckstrin | PHLDA2 | TAPAAPAEDAVAAAA | −1.42 | 0.21 |
| | homology-like | | AAPSEPSEPSRPpSPQP | | |
| | domain family A | | K | | |
| | member 2 | | (SEQ ID NO: 12) | | |
| | OS = Homo | | | | |
| | sapiens | | | | |
| | GN = PHLDA2 | | | | |
| | PE = 1 SV = 2 | | | | |
| Q96HC4 | PDZ and LIM | PDLIM5 | EVVKPVPIpTSPAVSK | −1.67 | −0.29 |
| | domain protein 5 | | (SEQ ID NO: 13) | | |
| | OS = Homo | | | | |
| | sapiens | | | | |
| | GN = PDLIM5 | | | | |
| | PE = 1 SV = 5 | | | | |
| O15446 | DNA-directed | CD3EAP | QEQINTEPLEDTVLpSP | −0.97 | 0.19 |
| | RNA polymerase | | TKK | | |
| | 1 subunit RPA34 | | (SEQ ID NO: 14) | | |
| | OS = Homo | | | | |
| | sapiens | | | | |
| | GN = CD3EAP | | | | |
| | PE = 1 SV = 1 | | | | |
| Q9NQS7 | Inner centromere | INCENP | HSPIAPSpSPSPQVLAQ | −1.40 | 0.03 |
| | protein | | K | | |
| | OS = Homo | | (SEQ ID NO: 15) | | |
| | sapiens | | | | |
| | GN = INCENP | | | | |
| | PE = 1 SV = 3 | | | | |
| P42858 | Huntingtin | HTT | EKEPGEQASVPLpSPK | −1.28 | −0.25 |
| | OS = Homo | | (SEQ ID NO: 16) | | |
| | sapiens | | | | |
| | GN = HTT PE = 1 | | | | |
| | SV = 2 | | | | |
| Q6IQ22 | Ras-related | RAB12 | AGGGGGLGAGpSPALS | −1.42 | −0.72 |
| | protein Rab-12 | | GGQGR | | |
| | OS = Homo | | (SEQ ID NO: 17) | | |
| | sapiens | | | | |
| | GN = RAB12 | | | | |
| | PE = 1 SV = 3 | | | | |
| Q9UGUO | Transcription | TCF20 | NCPAVTLTpSPAK | −0.96 | −0.47 |
| | factor 20 | | (SEQ ID NO: 18) | | |
| | OS = Homo | | | | |
| | sapiens | | | | |
| | GN = TCF20 | | | | |
| | PE = 1 SV = 3 | | | | |
| Q9NQS7 | Inner centromere | INCENP | HpSPIAPSpSPSPQVLA | −1.23 | −0.46 |
| | protein | | QK | | |
| | OS = Homo | | (SEQ ID NO: 19) | | |
| | sapiens | | | | |
| | GN = INCENP | | | | |
| | PE = 1 SV = 3 | | | | |
| E7EVAO | Microtubule- | MAP4 | DGVLTLANNVpTPAK | −1.11 | 0.85 |
| | associated | | (SEQ ID NO: 20) | | |
| | protein | | | | |
| | OS = Homo | | | | |
| | sapiens | | | | |
| | GN = MAP4 | | | | |
| | PE = 1 SV = 1 | | | | |
| Q8NEF9 | Serum response | SRFBP1 | DSVVSLESQKpTPADP | −1.92 | 0.31 |
| | factor-binding | | K | | |
| | protein 1 | | (SEQ ID NO: 21) | | |
| | OS = Homo | | | | |
| | sapiens | | | | |
| | GN = SRFBP1 | | | | |
| | PE = 1 SV = 1 | | | | |
| Q12830 | Nucleosome- | BPTF | PQVAAQSQPQSNVQG | −1.10 | −0.85 |
| | remodeling | | QpSPVR | | |
| | factor subunit | | (SEQ ID NO: 22) | | |
| | BPTF | | | | |
| | OS = Homo | | | | |
| | sapiens | | | | |
| | GN = BPTFPE = 1 | | | | |
| | SV = 3 | | | | |
| P49454 | Centromere | CENPF | SQQAAQSADVSLNPCN | −1.41 | 0.36 |
| | protein F | | pTPQK | | |
| | OS = Homo | | (SEQ ID NO: 23) | | |
| | sapiens | | | | |
| | GN = CENPF | | | | |
| | PE = 1 SV = 2 | | | | |
| P85037 | Forkhead box | FOXK1 | YSQSAPGpSPVSAQPVI | −1.24 | −1.53 |
| | protein K1 | | M(15.995)AVPPRPSSLV | | |
| | OS = Homo | | AK | | |
| | sapiens | | (SEQ ID NO: 24) | | |
| | GN = FOXK1 | | | | |
| | PE = 1 SV = 1 | | | | |
| J3QS41 | Probable | HELZ | GpSPIPYGLGHHPPVTI | −1.17 | 0.36 |
| | helicase with | | GQPQNQHQEK | | |
| | zinc finger | | (SEQ ID NO: 25) | | |
| | domain | | | | |
| | OS = Homo | | | | |
| | sapiens | | | | |
| | GN = HELZ | | | | |
| | PE = 1 SV = 1 | | | | |
| B5MBX0 | Sororin | CDCA5 | RIVAHAVEVPAVQpSP | −1.07 | 0.59 |
| | OS = Homo | | R | | |
| | sapiens | | (SEQ ID NO: 26) | | |
| | GN = CDCA5 | | | | |
| | PE = 1 SV = 1 | | | | |
| Q9H7N4 | Splicing factor, | SCAF1 | pSPSPAPAPAPAAAAG | −0.91 | −0.16 |
| | arginine/serine- | | PPTR | | |
| | rich 19 | | (SEQ ID NO: 27) | | |
| | OS = Homo | | | | |
| | sapiens | | | | |
| | GN = SCAF1 | | | | |
| | PE = 1 SV = 3 | | | | |
| Q5JSZ5 | Protein PRRC2B | PRRC2B | ApSPQENGPAVHK | −2.32 | −0.64 |
| | OS = Homo | | (SEQ ID NO: 28) | | |
| | sapiens | | | | |
| | GN = PRRC2B | | | | |
| | PE = 1 SV = 2 | | | | |
| Q8N8A6 | ATP-dependent | DDX51 | VNDAEPGpSPEAPQGK | −0.97 | 0.96 |
| | RNA helicase | | (SEQ ID NO: 29) | | |
| | DDX51 | | | | |
| | OS = Homo | | | | |
| | sapiens | | | | |
| | GN = DDX51 | | | | |
| | PE = 1 SV = 3 | | | | |
| Q5VT52-3 | Isoform 3 of | RPRD2 | NTGVSPASRPSPGpTPT | −1.32 | −0.09 |
| | Regulation of | | SPSNLTSGLK | | |
| | nuclear pre- | | (SEQ ID NO: 30) | | |
| | mRNA domain- | | | | |
| | containing | | | | |
| | protein 2 | | | | |
| | OS = Homo | | | | |
| | sapiens | | | | |
| | GN = RPRD2 | | | | |
| Q9Y6D5 | Brefeldin A- | ARFGEF2 | PQSPVIQAAAVpSPK | −1.08 | −0.69 |
| | inhibited guanine | | (SEQ ID NO: 31) | | |
| | nucleotide- | | | | |
| | exchange protein | | | | |
| | 2 OS = Homo | | | | |
| | sapiens | | | | |
| | GN = ARFGEF2 | | | | |
| | PE = 1 SV = 3 | | | | |
| Q9Y446 | Plakophilin-3 | PKP3 | AGGLDWPEATEVpSPS | −0.94 | 0.83 |
| | OS = Homo | | R | | |
| | sapiens | | (SEQ ID NO: 32) | | |
| | GN = PKP3 PE = 1 | | | | |
| | SV = 1 | | | | |
| Q9H7N4 | Splicing factor, | SCAF1 | pSPSPAPAPAPAAAAG | −0.99 | −0.53 |
| | arginine/serine- | | PPTRK | | |
| | rich 19 | | (SEQ ID NO: 33) | | |
| | OS = Homo | | | | |
| | sapiens | | | | |
| | GN = SCAF1 | | | | |
| | PE = 1 SV = 3 | | | | |
| Q6KC79 | Nipped-B-like | NIPBL | DVPPDILLDpSPERK | −0.86 | −0.06 |
| | protein | | (SEQ ID NO: 34) | | |
| | OS = Homo | | | | |
| | sapiens | | | | |
| | GN = NIPBL | | | | |
| | PE = 1 SV = 2 | | | | |
| Q12955-5 | Isoform 3 of | ANK3 | RYSYLTEPGM(15.995)S | −2.38 | 0.35 |
| | Ankyrin-3 | | PQpSPCER | | |
| | OS = Homo | | (SEQ ID NO: 35) | | |
| | sapiens | | | | |
| | GN = ANK3 | | | | |
| Q9UHG0 | Doublecortin | DCDC2 | STVGSSDNSpSPQPLK | −1.69 | −0.37 |
| | domain- | | (SEQ ID NO: 36) | | |
| | containing | | | | |
| | protein 2 | | | | |
| | OS = Homo | | | | |
| | sapiens | | | | |
| | GN = DCDC2 | | | | |
| | PE = 1 SV = 2 | | | | |
| Q9UHB7 | AF4/FMR2 | AFF4 | DLLPpSPAGPVPSK | −1.00 | −0.36 |
| | family member 4 | | (SEQ ID NO: 37) | | |
| | OS = Homo | | | | |
| | sapiens | | | | |
| | GN = AFF4 PE = 1 | | | | |
| | SV = 1 | | | | |
| Q96T58 | Msx2-interacting | SPEN | DSELKpTPPSVGPPSVT | −0.93 | −0.05 |
| | protein | | VVTLESAPSALEK | | |
| | OS = Homo | | (SEQ ID NO: 38) | | |
| | sapiens | | | | |
| | GN = SPEN PE = 1 | | | | |
| | SV = 1 | | | | |
| Q86UU0-4 | Isoform 4 of B- | BCL9L | TAM(15.995)PpSPGVSQ | −0.88 | 0.53 |
| | cell | | NK | | |
| | CLL/lymphoma | | (SEQ ID NO: 39) | | |
| | 9-like protein | | | | |
| | OS = Homo | | | | |
| | sapiens | | | | |
| | GN = BCL9L | | | | |
| Q8WUF5 | RelA-associated | PPP1R13 | AGpSPRGpSPLAEGPQA | −1.12 | 0.63 |
| | inhibitor | L | FFPER | | |
| | OS = Homo | | (SEQ ID NO: 40) | | |
| | sapiens | | | | |
| | GN = PPP1R13L | | | | |
| | PE = 1 SV = 4 | | | | |

### REFERENCES

The following References are incorporated by reference herein in their entireties:
[1] Akiyama, T., Ohuchi, T., Sumida, S., Matsumoto, K., and Toyoshima, K. (1992). Phosphorylation of the retinoblastoma protein by cdk2. Proc Natl Acad Sci USA 89, 7900-7904.
[2] Aktas, H., Cai, H., and Cooper, G. M. (1997). Ras links growth factor signaling to the cell cycle machinery via regulation of cyclin D1 and the Cdk inhibitor p27KIP1. Mol Cell Biol 17, 3850-3857.
[3] Aleem, E., Kiyokawa, H., and Kaldis, P. (2005). Cdc2-cyclin E complexes regulate the G1/S phase transition. Nat Cell Biol 7, 831-836.
[4] Arora, M., Moser, J., Phadke, H., Basha, A. A., and Spencer, S. L. (2017). Endogenous Replication Stress in Mother Cells Leads to Quiescence of Daughter Cells. Cell reports 19, 1351-1364.
[5] Baldin, V., Lukas, J., Marcote, M., Pagano, M., and Draetta, G. (1993). Cyclin D1 is a nuclear protein required for cell cycle progression in G1. Genes Dev 7, 812-821.
[6] Barriere, C., Santamaria, D., Cerqueira, A., Galan, J., Martin, A., Ortega, S., Malumbres, M., Dubus, P., and Barbacid, M. (2007). Mice thrive without Cdk4 and Cdk2. Mol Oncol 1, 72-83.
Berthet, C., Aleem, E., Coppola, V., Tessarollo, L., and Kaldis, P. (2003). Cdk2 knockout mice are viable. Curr Biol 13, 1775-1785.
[7] Brookes, S., Gagrica, S., Sanij, E., Rowe, J., Gregory, F. J., Hara, E., and Peters, G. (2015). Evidence for a CDK4-dependent checkpoint in a conditional model of cellular senescence. Cell Cycle 14, 1164-1173.
[8] Burkhart, D. L., and Sage, J. (2008). Cellular mechanisms of tumour suppression by the retinoblastoma gene. Nat Rev Cancer 8, 671-682.
[9] Caldon, C. E., Sergio, C. M., Kang, J., Muthukaruppan, A., Boersma, M. N., Stone, A., Barraclough, J., Lee, C. S., Black, M. A., Miller, L. D., et al. (2012). Cyclin E2 overexpression is associated with endocrine resistance but not insensitivity to CDK2 inhibition in human breast cancer cells. Mol Cancer Ther 11, 1488-1499.
[10] Cappell, S. D., Chung, M., Jaimovich, A., Spencer, S. L., and Meyer, T. (2016). Irreversible APC(Cdhl) Inactivation Underlies the Point of No Return for Cell-Cycle Entry. Cell 166, 167-180.
[11] Chellappan, S. P., Hiebert, S., Mudryj, M., Horowitz, J. M., and Nevins, J. R. (1991). The E2F transcription factor is a cellular target for the RB protein. Cell 65, 1053-1061.
[12] Chung, M., Liu, C., Yang, H. W., Koberlin, M. S., Cappell, S. D., and Meyer, T. (2019). Transient Hysteresis in CDK4/6 Activity Underlies Passage of the Restriction Point in G1. Mol Cell.
[13] Deshpande, A., Sicinski, P., and Hinds, P. W. (2005). Cyclins and cdks in development and cancer: a perspective. Oncogene 24, 2909-2915.
[14] Edwards, A., and Haas, W. (2016). Multiplexed Quantitative Proteomics for High-Throughput Comprehensive Proteome Comparisons of Human Cell Lines. Methods in molecular biology 1394, 1-13.
[15] Evron, E., Umbricht, C. B., Korz, D., Raman, V., Loeb, D. M., Niranjan, B., Buluwela, L., Weitzman, S. A., Marks, J., and Sukumar, S. (2001). Loss of cyclin D2 expression in the majority of breast cancers is associated with promoter hypermethylation. Cancer Res 61, 2782-2787.
[16] Fisk, H. A., and Winey, M. (2001). The mouse Mps1p-like kinase regulates centrosome duplication. Cell 106, 95-104.
[17] Franco, J., Witkiewicz, A. K., and Knudsen, E. S. (2014). CDK4/6 inhibitors have potent activity in combination with pathway selective therapeutic agents in models of pancreatic cancer. Oncotarget 5, 6512-6525.
[18] Gabrielli, B. G., Sarcevic, B., Sinnamon, J., Walker, G., Castellano, M., Wang, X. Q., and Ellem, K. A. (1999). A cyclin D-Cdk4 activity required for G2 phase cell cycle progression is inhibited in ultraviolet radiation-induced G2 phase delay. J Biol Chem 274, 13961-13969.
[19] Gookin, S., Min, M. W., Phadke, H., Chung, M. Y., Moser, J., Miller, I., Carter, D., and Spencer, S. L. (2017). A map of protein dynamics during cell-cycle progression and cell-cycle exit. Plos Biology 15.
[20] Grim, J. E., and Clurman, B. E. (2003). Cycling without CDK2? Trends Cell Biol 13, 396-399.
[21] Guerra, C., Mijimolle, N., Dhawahir, A., Dubus, P., Barradas, M., Serrano, M., Campuzano, V., and Barbacid, M. (2003). Tumor induction by an endogenous K-ras oncogene is highly dependent on cellular context. Cancer Cell 4, 111-120.
[22] Hata, A. N., Niederst, M. J., Archibald, H. L., Gomez-Caraballo, M., Siddiqui, F. M., Mulvey, H. E., Maruvka, Y. E., Ji, F., Bhang, H E., Krishnamurthy Radhakrishna, V., et al. (2016). Tumor cells can follow distinct evolutionary paths to become resistant to epidermal growth factor receptor inhibition. Nature medicine 22, 262-269.
[23] Herrera-Abreu, M. T., Palafox, M., Asghar, U., Rivas, M. A., Cutts, R. J., Garcia-Murillas, I., Pearson, A., Guzman, M., Rodriguez, O., Grueso, J., et al. (2016). Early Adaptation and Acquired Resistance to CDK4/6 Inhibition in Estrogen Receptor-Positive Breast Cancer. Cancer Res 76, 2301-2313.
[24] Hu, M. G., Deshpande, A., Enos, M., Mao, D., Hinds, E. A., Hu, G. F., Chang, R., Guo, Z., Dose, M., Mao, C., et al. (2009). A requirement for cyclin-dependent kinase 6 in thymocyte development and tumorigenesis. Cancer Res 69, 810-818.
[25] Huttlin, E. L., Jedrychowski, M. P., Elias, J. E., Goswami, T., Rad, R., Beausoleil, S. A., Villen, J., Haas, W., Sowa, M. E., and Gygi, S. P. (2010). A tissue-specific atlas of mouse protein phosphorylation and expression. Cell 143, 1174-1189.
[26] Jonkers, J., Meuwissen, R., van der Gulden, H., Peterse, H., van der Valk, M., and Berns, A. (2001). Synergistic tumor suppressor activity of BRCA2 and p53 in a conditional mouse model for breast cancer. Nat Genet 29, 418-425.
[27] Kato, J., Matsushime, H., Hiebert, S. W., Ewen, M. E., and Sherr, C. J. (1993). Direct binding of cyclin D to the retinoblastoma gene product (pRb) and pRb phosphorylation by the cyclin D-dependent kinase CDK4. Genes Dev 7, 331-342.
[28] Katsuno, Y., Suzuki, A., Sugimura, K., Okumura, K., Zineldeen, D. H., Shimada, M., Niida, H., Mizuno, T., Hanaoka, F., and Nakanishi, M. (2009). Cyclin A-Cdk1 regulates the origin firing program in mammalian cells. Proc Natl Acad Sci USA 106, 3184-3189.
[29] Keyomarsi, K., Tucker, S. L., Buchholz, T. A., Callister, M., Ding, Y., Hortobagyi, G. N., Bedrosian, I., Knickerbocker, C., Toyofuku, W., Lowe, M., et al. (2002). Cyclin E and survival in patients with breast cancer. N Engl J Med 347, 1566-1575.
[30] Khatib, Z. A., Matsushime, H., Valentine, M., Shapiro, D. N., Sherr, C. J., and Look, A. T. (1993). Coamplification of the CDK4 gene with MDM2 and GLI in human sarcomas. Cancer Res 53, 5535-5541.
[31] Lapek, J. D., Jr., Greninger, P., Morris, R., Amzallag, A., Pruteanu-Malinici, I., Benes, C. H., and Haas, W. (2017a). Detection of dysregulated protein-association networks by high-throughput proteomics predicts cancer vulnerabilities. Nature biotechnology 35, 983-989.
[32] Lapek, J. D., Jr., Lewinski, M. K., Wozniak, J. M., Guatelli, J., and Gonzalez, D. J. (2017b). Quantitative Temporal Viromics of an Inducible HIV-1 Model Yields Insight to Global Host Targets and Phospho-Dynamics Associated with Protein Vpr. Molecular & cellular proteomics: MCP 16, 1447-1461.
[33] Lee, C. L., Moding, E. J., Huang, X., Li, Y., Woodlief, L. Z., Rodrigues, R. C., Ma, Y., and Kirsch, D. G. (2012). Generation of primary tumors with Flp recombinase in FRT-flanked p53 mice. Dis Model Mech 5, 397-402.
[34] Lindqvist, A., Rodriguez-Bravo, V., and Medema, R. H. (2009). The decision to enter mitosis: feedback and redundancy in the mitotic entry network. Journal of Cell Biology 185, 193-202.
[35] Lohka, M. J., Hayes, M. K., and Maller, J. L. (1988). Purification of maturation-promoting factor, an intracellular regulator of early mitotic events. Proc Natl Acad Sci USA 85, 3009-3013.
[36] Lundberg, A. S., and Weinberg, R. A. (1998). Functional inactivation of the retinoblastoma protein requires sequential modification by at least two distinct cyclin-cdk complexes. Molecular and cellular biology 18, 753-761.
[37] Malumbres, M., Sotillo, R., Santamaria, D., Galan, J., Cerezo, A., Ortega, S., Dubus, P., and Barbacid, M. (2004). Mammalian cells cycle without the D-type cyclin-dependent kinases Cdk4 and Cdk6. Cell 118, 493-504.
[38] Massague, J. (2004). G1 cell-cycle control and cancer. Nature 432, 298-306.
[39] Matsushime, H., Ewen, M. E., Strom, D. K., Kato, J. Y., Hanks, S. K., Roussel, M. F., and Sherr, C. J. (1992). Identification and properties of an atypical catalytic subunit (p34PSK-J3/cdk4) for mammalian D type G1 cyclins. Cell 71, 323-334.
[40] Matsushime, H., Quelle, D. E., Shurtleff, S. A., Shibuya, M., Sherr, C., and Kato, J. Y. (1994). D-type cyclin-dependent kinase activity in mammalian cells. Mol Cell Biol 14, 2066-2076.
[41] Merrick, K. A., Wohlbold, L., Zhang, C., Allen, J. J., Horiuchi, D., Huskey, N. E., Goga, A., Shokat, K. M., and Fisher, R. P. (2011). Switching Cdk2 On or Off with Small Molecules to Reveal Requirements in Human Cell Proliferation. Molecular Cell 42, 624-636.
[42] Meyerson, M., and Harlow, E. (1994). Identification of G1 kinase activity for cdk6, a novel cyclin D partner. Mol Cell Biol 14, 2077-2086.
[43] Mittnacht, S., Lees, J. A., Desai, D., Harlow, E., Morgan, D. O., and Weinberg, R. A. (1994). Distinct sub-populations of the retinoblastoma protein show a distinct pattern of phosphorylation. EMBO J 13, 118-127.
[44] Moons, D. S., Jirawatnotai, S., Parlow, A. F., Gibori, G., Kineman, R. D., and Kiyokawa, H. (2002). Pituitary hypoplasia and lactotroph dysfunction in mice deficient for cyclin-dependent kinase-4. Endocrinology 143, 3001-3008.
[45] Musgrove, E. A., Caldon, C. E., Barraclough, J., Stone, A., and Sutherland, R. L. (2011). Cyclin D as a therapeutic target in cancer. Nat Rev Cancer 11, 558-572.
[46] Narasimha, A. M., Kaulich, M., Shapiro, G. S., Choi, Y. J., Sicinski, P., and Dowdy, S. F. (2014). Cyclin D activates the Rb tumor suppressor by mono-phosphorylation. Elife 3. Ohtani, K., DeGregori, J., and Nevins, J. R. (1995). Regulation of the cyclin E gene by transcription factor E2F1. Proc Natl Acad Sci USA 92, 12146-12150.
[47] Okuda, M., Horn, H. F., Tarapore, P., Tokuyama, Y., Smulian, A. G., Chan, P. K., Knudsen, E. S., Hofmann, I. A., Snyder, J. D., Bove, K. E., et al. (2000). Nucleophosmin/B23 is a target of CDK2/cyclin E in centrosome duplication. Cell 103, 127-140.
[48] Ortega, S., Prieto, I., Odajima, J., Martin, A., Dubus, P., Sotillo, R., Barbero, J. L., Malumbres, M., and Barbacid, M. (2003). Cyclin-dependent kinase 2 is essential for meiosis but not for mitotic cell division in mice. Nat Genet 35, 25-31.
[49] Park, S., Lee, J., Do, I. G., Jang, J., Rho, K., Ahn, S., Maruja, L., Kim, S. J, Kim, K. M., Mao, M., et al. (2014). Aberrant CDK4 amplification in refractory rhabdomyosarcoma as identified by genomic profiling. Sci Rep 4, 3623.
[50] Petersen, B. O., Lukas, J., Sorensen, C. S., Bartek, J., and Helin, K. (1999). Phosphorylation of mammalian CDC6 by cyclin A/CDK2 regulates its subcellular localization. EMBO J 18, 396-410.
[51] Ramirez, M., Rajaram, S., Steininger, R. J., Osipchuk, D., Roth, M. A., Morinishi, L. S., Evans, L., Ji, W., Hsu, C. H., Thurley, K., et al. (2016). Diverse drug-resistance mechanisms can emerge from drug-tolerant cancer persister cells. Nature communications 7, 10690.
[52] Rane, S. G., Dubus, P., Mettus, R. V., Galbreath, E. J., Boden, G., Reddy, E. P., and Barbacid, M. (1999). Loss of Cdk4 expression causes insulin-deficient diabetes and Cdk4 activation results in beta-islet cell hyperplasia. Nat Genet 22, 44-52.
[53] Saha, P., Chen, J., Thome, K. C., Lawlis, S. J., Hou, Z. H., Hendricks, M., Parvin, J. D., and Dutta, A. (1998). Human CDC6/Cdc18 associates with Orc1 and cyclin-cdk and is selectively eliminated from the nucleus at the onset of S phase. Mol Cell Biol 18, 2758-2767.
[54] Sanidas, I., Morris, R., Fella, K. A., Rumde, P. H., Boukhali, M., Tai, E. C., Ting, D. T., Lawrence, M. S., Haas, W., and Dyson, N.J. (2019). A Code of Mono-phosphorylation Modulates the Function of RB. Mol Cell 73, 985-1000 e1006.
[55] Santamaria, D., Barriere, C., Cerqueira, A., Hunt, S., Tardy, C., Newton, K., Caceres, J. F., Dubus, P., Malumbres, M., and Barbacid, M. (2007). Cdk1 is sufficient to drive the mammalian cell cycle. Nature 448, 811-815.
[56] Sarcevic, B., Lilischkis, R., and Sutherland, R. L. (1997). Differential phosphorylation of T-47D human breast cancer cell substrates by D1-, D3-, E-, and A-type cyclin-CDK complexes. J Biol Chem 272, 33327-33337.
[57] Schwarz, C., Johnson, A., Koivomagi, M., Zatulovskiy, E., Kravitz, C. J., Doncic, A., and Skotheim, J. M. (2018). A Precise Cdk Activity Threshold Determines Passage through the Restriction Point. Mol Cell 69, 253-264 e255.
[58] Shaffer, S. M., Dunagin, M. C., Torborg, S. R., Torre, E. A., Emert, B., Krepler, C., Beqiri, M., Sproesser, K., Brafford, P. A., Xiao, M., et al. (2017). Rare cell variability and drug-induced reprogramming as a mode of cancer drug resistance. Nature 546, 431-435.
[59] Sharma, S. V., Lee, D. Y., Li, B., Quinlan, M. P., Takahashi, F., Maheswaran, S., McDermott, U., Azizian, N., Zou, L., Fischbach, M. A., et al. (2010). A chromatin-mediated reversible drug-tolerant state in cancer cell subpopulations. Cell 141, 69-80.
[60] Sherr, C. J. (1993). Mammalian G1 cyclins. Cell 73, 1059-1065.
[61] Sherr, C. J. (1994). G1 phase progression: cycling on cue. Cell 79, 551-555.
[62] Sherr, C. J., and Roberts, J. M. (2004). Living with or without cyclins and cyclin-dependent kinases. Genes Dev 18, 2699-2711.
[63] Spencer, S. L., Cappell, S. D., Tsai, F. C., Overton, K. W., Wang, C. L., and Meyer, T. (2013). The proliferation-quiescence decision is controlled by a bifurcation in CDK2 activity at mitotic exit. Cell 155, 369-383.
[64] Tetsu, O., and McCormick, F. (2003). Proliferation of cancer cells despite CDK2 inhibition. Cancer Cell 3, 233-245.
[65] Wohlbold, L., Merrick, K. A., De, S., Amat, R., Kim, J. H., Larochelle, S., Allen, J. J., Zhang, C., Shokat, K. M., Petrini, J. H., et al. (2012). Chemical genetics reveals a specific requirement for Cdk2 activity in the DNA damage response and identifies Nbs1 as a Cdk2 substrate in human cells. PLoS Genet 8, e1002935.
[66] Xu, T., Park, S. K., Venable, J. D., Wohlschlegel, J. A., Diedrich, J. K., Cociorva, D., Lu, B., Liao, L., Hewel, J., Han, X., et al. (2015). ProLuCID: An improved SEQUEST-like algorithm with enhanced sensitivity and specificity. Journal of proteomics 129, 16-24.
[67] Yang, C., Li, Z., Bhatt, T., Dickler, M., Gin, D., Scaltriti, M., Baselga, J., Rosen, N., and Chandarlapaty, S. (2017a). Acquired CDK6 amplification promotes breast cancer resistance to CDK4/6 inhibitors and loss of ER signaling and dependence. Oncogene 36, 2255-2264.
[68] Yang, H. W., Chung, M., Kudo, T., and Meyer, T. (2017b). Competing memories of mitogen and p53 signalling control cell-cycle entry. Nature 549, 404-408.
[69] Yang, K., Hitomi, M., and Stacey, D. W. (2006). Variations in cyclin D1 levels through the cell cycle determine the proliferative fate of a cell. Cell Div 1.
[70] Zerjatke, T., Gak, I. A., Kirova, D., Fuhrmann, M., Daniel, K., Gonciarz, M., Muller, D., Glauche, I., and Mansfeld, J. (2017). Quantitative Cell Cycle Analysis Based on an Endogenous All-in-One Reporter for Cell Tracking and Classification. Cell Reports 19, 1953-1966.
[71] Zhao, J., Kennedy, B. K., Lawrence, B. D., Barbie, D. A., Matera, A. G., Fletcher, J. A., and Harlow, E. (2000). NPAT links cyclin E-Cdk2 to the regulation of replication-dependent histone gene transcription. Genes Dev 14, 2283-2297.

1. A method for treating cancer comprising administering to a subject in need thereof a therapeutically effective amount of a CDK2 inhibitor, and a therapeutically effective amount of a CDK4/6 inhibitor, wherein the therapeutically effective amounts together are effective in treating cancer.

2. The method of claim 1, wherein the cancer is characterized by dependence on CDK2 for tumor cell proliferation.

3. The method of claim 1, wherein the therapeutically effective amount of the CDK4/6 inhibitor prevents rebound phosphorylation mediated by CDK4 and/or CDK6 in response to the inhibition of CDK2.

4. The method of claim 1, wherein the CDK4/6 inhibitor is selected from the group consisting of: abemaciclib, ribociclib, palbociclib, lerociclib, trilaciclib, SHR-6390, and BPI-16350, or a pharmaceutically acceptable salt thereof.

5. The method of claim 1, wherein the CDK2 inhibitor is selected from the group consisting of: 6-(difluoromethyl)-8-[(1R,2R)-2-hydroxy-2-methylcyclopentyl]-2-{[1-(methylsulfonyl)piperidin-4-yl]amino}pyrido[2,3-d]pyrimidin-7(8H)-one (PF-06873600), milciclib, inditinib, and FN-1501, or a pharmaceutically acceptable salt thereof.

6. The method of claim 1, wherein the CDK2 inhibitor and the CDK4/6 inhibitor are administered sequentially, concurrently or simultaneously.

7. The method of claim 1, wherein the CDK2 inhibitor is PF-06873600 or a pharmaceutically acceptable salt thereof.

8. The method of claim 1, wherein the CDK4/6 inhibitor is palbociclib or a pharmaceutically acceptable salt thereof.

9. A method for inhibiting rebound phosphorylation mediated by CDK4 and/or CDK6 in response to the inhibition of CDK2 in a cell comprising introducing to the cell an amount of a CDK2 inhibitor and an amount of a CDK4/6 inhibitor, wherein the amount of the CDK4/6 inhibitor is effective in inhibiting rebound phosphorylation mediated by CDK4 and/or CDK6 in response to the inhibition of CDK2.

10. The method of claim 9, wherein the cell is a cancer cell.

11. The method of claim 10, wherein the cancer cell is characterized by dependence on CDK2 for tumor cell proliferation.

12. The method of claim 9, wherein the CDK4/6 inhibitor is selected from the group consisting of: abemaciclib, ribociclib, palbociclib, lerociclib, trilaciclib, SHR-6390, and BPI-16350, or a pharmaceutically acceptable salt thereof.

13. The method of claim 9, wherein the CDK2 inhibitor is selected from the group consisting of: PF-06873600, milciclib, inditinib, and FN-1501, or a pharmaceutically acceptable salt thereof.

14. The method of claim 9, wherein the CDK2 inhibitor and the CDK4/6 inhibitor are administered sequentially, concurrently or simultaneously to a subject in need thereof.

15. The method of claim 9, wherein the CDK2 inhibitor is PF-06873600 or a pharmaceutically acceptable salt thereof.

16. The method of claim 9, wherein the CDK4/6 inhibitor is palbociclib or a pharmaceutically acceptable salt thereof.


SEQUENCE LISTING (40 sequences)

SEQ ID NO: 1 - protein, 16 aa, Homo sapiens
  MOD_RES_PHOSPHORYLATION (11)..(11): Phosphorylation on Threonine
DVPPLSETEATPVPIK

SEQ ID NO: 2 - protein, 24 aa, Homo sapiens
  MOD_RES_PHOSPHORYLATION (21)..(21): Phosphorylation on Threonine
GIASTSDPPTANIKPTPVVSTPSK

SEQ ID NO: 3 - protein, 13 aa, Homo sapians
  MOD_RES_PHOSPHORYLATION (10)..(10): Phosphorylation on Threonine
SVSSVLSSLTPAK

SEQ ID NO: 4 - protein, 17 aa, Homo sapiens
  MOD_RES_PHOSPHORYLATION (14)..(14): Phosphorylation on Serine
ELPSLSPAPDTGLSPSK

SEQ ID NO: 5 - protein, 18 aa, Homo sapiens
  MOD_RES_PHOSPHORYLATION (13)..(13): Phosphorylation on Serine
AGVVNGTGAPGQSPGAGR

SEQ ID NO: 6 - protein, 13 aa, Homo sapiens
  MOD_RES_PHOSPHORYLATION (10)..(10): Phosphorylation on Serine
VNFPENGFLSPDK

SEQ ID NO: 7 - protein, 14 aa, Homo sapiens
  MOD_RES_PHOSPHORYLATION (11)..(11): Phosphorylation on Serine
RVIAIDSDAESPAK

SEQ ID NO: 8 - protein, 16 aa, Homo sapiens
  MOD_RES_PHOSPHORYLATION (14)..(14): Phosphorylation on Threonine
GLSQNQQIPQNSVTPR

SEQ ID NO: 9 - protein, 14 aa, Homo sapiens
  MOD_RES_PHOSPHORYLATION (12)..(12): Phosphorylation on Serine
ISMQDVDLSLGSPK

SEQ ID NO: 10 - protein, 15 aa, Homo sapiens
  MOD_RES_PHOSPHORYLATION (14)..(14): Phosphorylation on Serine
GGVTGSPEASISGSK

SEQ ID NO: 11 - protein, 14 aa, Homo sapiens
  MOD_RES_PHOSPHORYLATION (3)..(3): Phosphorylation on Threonine
TNTPQGVLPSSQLK

SEQ ID NO: 12 - protein, 32 aa, Homo sapiens
  MOD_RES_PHOSPHORYLATION (28)..(28): Phosphorylation on Serine
TAPAAPAEDAVAAAAAAPSEPSEPSRPSPQPK

SEQ ID NO: 13 - protein, 15 aa, Homo sapiens
  MOD_RES_PHOSPHORYLATION (9)..(9): Phosphorylation on Threonine
EVVKPVPITSPAVSK

SEQ ID NO: 14 - protein, 19 aa, Homo sapiens
  MOD_RES_PHOSPHORYLATION (15)..(15): Phosphorylation on Serine
QEQINTEPLEDTVLSPTKK

SEQ ID NO: 15 - protein, 17 aa, Homo sapiens
  MOD_RES_PHOSPHORYLATION (8)..(8): Phosphorylation on Serine
HSPIAPSSPSPQVLAQK

SEQ ID NO: 16 - protein, 15 aa, Homo sapiens
  MOD_RES_PHOSPHORYLATION (13)..(13): Phosphorylation on Serine
EKEPGEQASVPLSPK

SEQ ID NO: 17 - protein, 20 aa, Homo sapiens
  MOD_RES_PHOSPHORYLATION (11)..(11): Phosphorylation on Serine
AGGGGGLGAGSPALSGGQGR

SEQ ID NO: 18 - protein, 12 aa, Homo sapiens
  MOD_RES_PHOSPHORYLATION (9)..(9): Phosphorylation on Serine
NCPAVTLTSPAK

SEQ ID NO: 19 - protein, 17 aa, Homo sapiens
  MOD_RES_PHOSPHORYLATION (2)..(2): Phosphorylation on Serine
  MOD_RES_PHOSPHORYLATION (8)..(8): Phosphorylation on Serine
HSPIAPSSPSPQVLAQK

SEQ ID NO: 20 - protein, 14 aa, Homo sapiens
  MOD_RES_PHOSPHORYLATION (11)..(11): Phosphorylation on Threonine
DGVLTLANNVTPAK

SEQ ID NO: 21 - protein, 16 aa, Homo sapiens
  MOD_RES_PHOSPHORYLATION (11)..(11): Phosphorylation on Threonine
DSVVSLESQKTPADPK

SEQ ID NO: 22 - protein, 20 aa, Homo sapiens
  MOD_RES_PHOSPHORYLATION (17)..(17): Phosphorylation on Serine
PQVAAQSQPQSNVQGQSPVR

SEQ ID NO: 23 - protein, 20 aa, Homo sapiens
  MOD_RES_PHOSPHORYLATION (17)..(17): Phosphorylation on Threonine
SQQAAQSADVSLNPCNTPQK

SEQ ID NO: 24 - protein, 29 aa, Homo sapiens
  MOD_RES_PHOSPHORYLATION (8)..(8): Phosphorylation on Serine
  M(15.995) (17)..(17): Oxidation on Methionine
YSQSAPGSPVSAQPVIMAVPPRPSSLVAK

SEQ ID NO: 25 - protein, 26 aa, Homo sapiens
  MOD_RES_PHOSPHORYLATION (2)..(2): Phosphorylation on Serine
GSPIPYGLGHHPPVTIGQPQNQHQEK

SEQ ID NO: 26 - protein, 16 aa, Homo sapiens
  MOD_RES_PHOSPHORYLATION (14)..(14): Phosphorylation on Serine
RIVAHAVEVPAVQSPR

SEQ ID NO: 27 - protein, 19 aa, Homo sapiens
  MOD_RES_PHOSPHORYLATION (1)..(1): Phosphorylation on Serine
SPSPAPAPAPAAAAGPPTR

SEQ ID NO: 28 - protein, 12 aa, Homo sapiens
  MOD_RES_PHOSPHORYLATION (2)..(2): Phosphorylation on Serine
ASPQENGPAVHK

SEQ ID NO: 29 - protein, 15 aa, Homo sapiens
  MOD_RES_PHOSPHORYLATION (8)..(8): Phosphorylation on Serine
VNDAEPGSPEAPQGK

SEQ ID NO: 30 - protein, 26 aa, Homo sapiens
  MOD_RES_PHOSPHORYLATION (14)..(14): Phosphorylation on Threonine
NTGVSPASRPSPGTPTSPSNLTSGLK

SEQ ID NO: 31 - protein, 14 aa, Homo sapiens
  MOD_RES_PHOSPHORYLATION (12)..(12): Phosphorylation on Serine
PQSPVIQAAAVSPK

SEQ ID NO: 32 - protein, 16 aa, Homo sapiens
  MOD_RES_PHOSPHORYLATION (13)..(13): Phosphorylation on Serine
AGGLDWPEATEVSPSR

SEQ ID NO: 33 - protein, 20 aa, Homo sapiens
  MOD_RES_PHOSPHORYLATION (1)..(1): Phosphorylation on Serine
SPSPAPAPAPAAAAGPPTRK

SEQ ID NO: 34 - protein, 14 aa, Homo sapiens
  MOD_RES_PHOSPHORYLATION (10)..(10): Phosphorylation on Serine
DVPPDILLDSPERK

SEQ ID NO: 35 - protein, 18 aa, Homo sapiens
  MOD_RES (10)..(10): Oxidation on Methionine
  MOD_RES_PHOSPHORYLATION (14)..(14): Phosphorylation on Serine
RYSYLTEPGMSPQSPCER

SEQ ID NO: 36 - protein, 15 aa, Homo sapiens
  MOD_RES_PHOSPHORYLATION (10)..(10): Phosphorylation on Serine
STVGSSDNSSPQPLK

SEQ ID NO: 37 - protein, 13 aa, Homo sapiens
  MOD_RES_PHOSPHORYLATION (5)..(5): Phosphorylation on Serine
DLLPSPAGPVPSK

SEQ ID NO: 38 - protein, 23 aa, Homo sapiens
  MOD_RES_PHOSPHORYLATION (6)..(6): Phosphorylation on Threonine
DSELKTPPSVGPPSVTVVTLESA

SEQ ID NO: 39 - protein, 12 aa, Homo sapiens
  MOD_RES (3)..(3): Oxidation on Methionine
  MOD_RES_PHOSPHORYLATION (5)..(5): Phosphorylation on Serine
TAMPSPGVSQNK

SEQ ID NO: 40 - protein, 20 aa, Homo sapiens
  MOD_RES_PHOSPHORYLATION (3)..(3): Phosphorylation on Serine
  MOD_RES_PHOSPHORYLATION (7)..(7): Phosphorylation on Serine
AGSPRGSPLAEGPQAFFPER